(19) United States
(12) Patent Application Publication
(10) Pub. No.: US 2026/0365662 A1
(43) Pub. Date: Nov. 19, 2026

(54) ISOANDROGRAPHOLIDE ANALOGS AND METHOD FOR TREATMENT USING THE SAME

(71) Applicant: Enveda Therapeutics, Inc., Boulder, CO (US)

(72) Inventors: Subbaraju Venkata GOTTUMUKKALA, Visakhapatnam (IN); Kiran CHERUKUPALLI, Visakhapatnam (IN); Bryan Hurst NORMAN, Boulder, CO (US); Prabhakar K. JADHAV, Boulder, CO (US); Sotirios K. KARATHANASIS, Boulder, CO (US); Goutam MANDAL, Boulder, CO (US); Indranil MUKHOPADHYAY, Visakhapatnam (IN)

(21) Appl. No.: 19/485,473

(22) PCT Filed: May 17, 2024
(86) PCT No.: PCT/US2024/030111

§ 371 Date: Nov. 18, 2025

Related U.S. Application Data
(60) Provisional application No. 63/503,156, filed on May 18, 2023.

(51) Int. Cl. C07D 491/048; A61K 31/365; A61K 31/4162; A61K 31/424; A61K 31/496; A61K 31/5377; A61K 31/55; A61P 1/00; A61P 11/00; A61P 17/00; A61P 17/06; A61P 29/00; C07D 307/92; C07D 493/04; C07D 498/04

(52) U.S. Cl. CPC C07D 491/048; A61K 31/365; A61K 31/4162; A61K 31/424; A61K 31/496; A61K 31/5377; A61K 31/55; A61P 1/00; A61P 11/00; A61P 17/00; A61P 17/06; A61P 29/00; C07D 307/92; C07D 493/04; C07D 498/04

(57) ABSTRACT

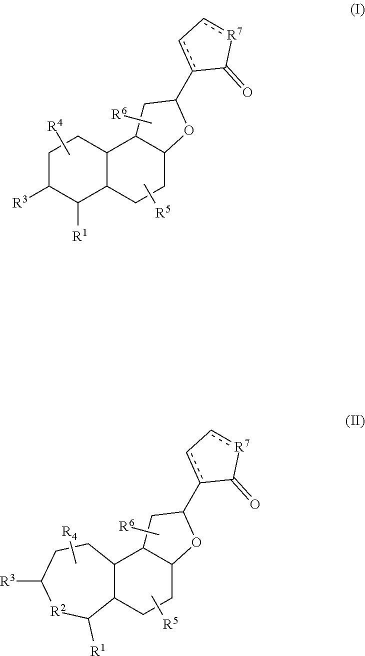

The present disclosure relates generally to compounds, compositions, and methods for treating a variety of diseases and conditions using a therapeutically effective amount of a compound of Formula (I) or Formula (II), a pharmaceutically acceptable salt, or derivative thereof.

### CROSS-REFERENCE TO RELATED APPLICATIONS

This application claims priority and benefit of U.S. Provisional Patent Application No. 63/503,156 filed on May 18, 2023, the contents of which are incorporated herein by reference in their entirety for all purposes.

### TECHNICAL FIELD

The present disclosure relates generally to compounds, compositions, and methods for treating a variety of diseases and conditions, and more specifically, for treating fibrosis and atopic dermatitis (AD), along with a variety of other diseases and conditions relating to neutrophil migration.

### BACKGROUND

Fibrosis is a chronic and progressive condition defined by an excessive accumulation of extracellular matrix (ECM) that causes tissue stiffness, or scarring. It develops through complex interactions between cells, extracellular matrix, cytokines, and growth factors. Different cell types are involved, including resident mesenchymal cells (fibroblasts and myofibroblasts) and ECM-producing cells derived from epithelial and endothelial cells (via a process known as epithelial- and endothelial-mesenchymal transition), as well as local or bone marrow-derived stem cells (fibrocytes). Myofibroblasts have long been regarded as the principal effector cell in fibrogenesis and the major cell type involved in normal wound healing. In animal models of fibrosis, the presence of myofibroblasts in fibrotic lesions corresponds with the development of active fibrosis, and their persistence and localization to fibrotic foci in human disease is correlated with disease progression. Moreover, myofibroblasts have an improved migratory behavior and are capable of releasing a variety of pro-fibrotic mediators.

Fibrotic illnesses, such as idiopathic pulmonary fibrosis, systemic sclerosis, liver cirrhosis, and progressive kidney disease, are a primary source of morbidity and mortality and are capable of affecting all tissues and organs. In addition to influencing cancer metastasis, fibrotic tissue remodeling can exacerbate chronic graft rejection in transplant recipients.

With particular attention to pulmonary disorders, inflammation of the airways is a hallmark symptom of chronic inflammatory lung disorders, which are almost always linked with unfavorable clinical outcomes. Airway neutrophilia is a trait that is shared by all of these disorders, despite the fact that their causes are quite diverse. In the immune system's defense of the lung, mechanisms such as phagocytosis, degranulation, neutrophil extracellular trap formation, exosome secretion, release of cytokines and chemokines, and autophagy are all involved. Neutrophils, as vital regulators linking the innate and adaptive immune systems, are a double-edged sword. Even though neutrophils are an effective line of defense against external pathogens, neutrophils and the components that make up neutrophils have the potential to set off a number of cascades that result in inflammation and fibrogenesis.

Eczema is a chronic disease that causes inflammation, redness, and irritation of the skin. Atopic dermatitis is the most common form of eczema. The causes of eczema, including atopic dermatitis, remain unclear. Atopic dermatitis is a major global public health problem, affecting 1%-20% of people worldwide. The prevalence of atopic dermatitis in adults is about 1%-3%, and 10%-20%, in children. Atopic dermatitis is the most common form of eczema in childhood. Since 1960s, the prevalence of atopic dermatitis has increased more than 3-fold. The reasons for the rising prevalence are as yet unclear (Lee, J. H., et al. A Comprehensive Review of the Treatment of Atopic Eczema, Allergy Asthma Immunol. Res., 2016 8(3), 181-190). Atopic dermatitis is a worldwide public health concern with significant financial burden. In addition, atopic dermatitis affects the quality of life of families as well as those affected themselves. Eczema, including atopic dermatitis, remains a challenging condition for physicians and patients.

Accordingly, there is an ongoing need in the art for compounds, compositions and methods for treating fibrosis, such as idiopathic pulmonary fibrosis (IPF), and atopic dermatitis, along with a variety of other diseases and conditions relating to neutrophil migration.

### BRIEF SUMMARY

Embodiments described herein relate to compounds, compositions, and methods for treating and/or preventing fibrosis and various fibrotic diseases, disorders or conditions. It has been found that compounds described herein, such as a compound of Formula (I) and (II), can be administered to a subject in need thereof to decrease fibrotic symptoms, such as collagen deposition, collagen accumulation, collagen fiber formation, inflammatory cytokine expression, and inflammatory cell infiltration, and treat and/or prevent various fibrotic diseases, disorders, and conditions characterized, in whole or in part, by the excess production of fibrous material, including excess production of fibrotic material within the extracellular matrix, or the replacement of normal tissue elements by abnormal, non-functional, and/or excessive accumulation of matrix-associated components.

In other embodiments, it has been found that compounds described herein, such as a compound of Formula (I) and (II), can be administered to a subject in need thereof to decrease atopic dermatitis symptoms and treat and/or prevent various dermatological conditions, such as atopic dermatitis.

A compound of Formula (I) or Formula (II) is provided herein, as follows:

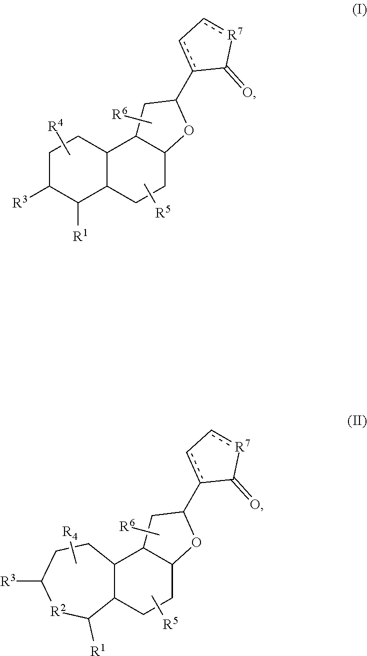

or pharmaceutically acceptable salts thereof, wherein:
R¹ is a alkyl, —CN, —NH₂, —OH, —CH₂OH, —CHO, —CH₂R⁸, —COOH, —CONH₂, —COR⁸, —N₃, —COR⁸, —SH—S(alkyl), halogen, aryl, heteroaryl, or linked to R³ to form a 3- to 10-membered cyclic or heterocyclic group, wherein the alkyl, the aryl, the heteroaryl, and the cyclic or heterocyclic group are unsubstituted or substituted with one or more R⁸;
R² is oxygen or NR⁹;
R³ is a hydroxyl, ═O, halogen, —SH, —S(alkyl), —NR⁹R¹⁰, —COOH, —COOR⁹, —CONH₂, —CN, —N₃, haloalkyl, or alkoxy;
R⁴, R⁵, and R⁶ are each, independently, hydrogen or an unsubstituted or substituted alkyl;
R⁷ is oxygen, nitrogen, or NR⁹;
each R⁸ is, independently, hydroxyl, halogen, —SH, —S(alkyl), —NR⁹R¹⁰, —COOH, —COOR⁹, —CONH₂, —COR⁹, —COHR⁹R¹⁰, —CN, —N₃, alkyl, haloalkyl, alkenyl, alkynyl, alkoxy, aryl, or aralkyl; and
R⁹ and R¹⁰ are each, independently, hydrogen or an unsubstituted or substituted alkyl; with the proviso that the compound of Formula (I) is not isoandrographolide.

In some embodiments, a method of treating or preventing a fibrotic disease, disorder or condition includes administering to a subject in need thereof a therapeutically effect amount of compound of Formula (I) or (II).

In certain embodiments, the compound of Formula (I) is Formula (Ia) and the compound of Formula (II) is Formula (IIa), as follows:

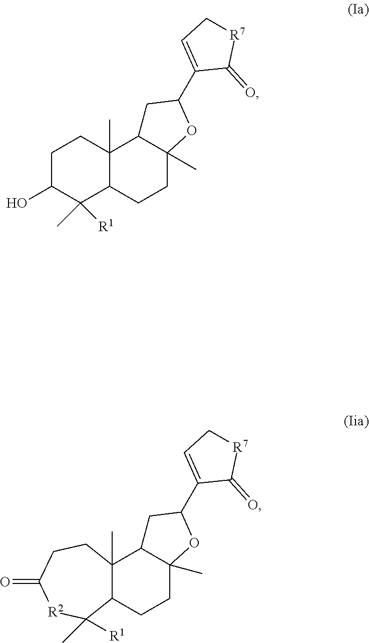

or pharmaceutically acceptable salts thereof, wherein:
R¹ is a C₁-C₆ alkyl, —CN, —NH₂, —OH, —CH₂OH, CHO, —CH₂R⁸, —COOH, —CONH₂, —COR⁸, —N₃, —COR⁸, —SH—Sme, a halogen atom, aryl, heteroaryl, or linked to R³ to form a 3- to 10-membered cyclic or heterocyclic group, wherein the C₁-C₆ alkyl, the aryl, the heteroaryl, and the cyclic or heterocyclic group are unsubstituted or substituted with one or more R⁸;
R² is oxygen or NR⁹;
R⁷ is oxygen, nitrogen, or NR⁹;
each R⁸ is, independently, a hydroxyl, a halogen, —SH, —Sme, —NR⁹R¹⁰, —COOH, —COOR⁹, —CONH₂, —COR⁹, —COHR⁹R¹⁰, —CN, —N₃, C₁-C₅ alkyl, C₁-C₅ haloalkyl, C₂-C₅ alkenyl, C₂-C₅ alkynyl, C₁-C₅ alkoxy, aryl, or aralkyl; and R⁹ and R¹⁰ are each, independently, hydrogen or a C₁-C₅ unsubstituted or substituted alkyl;
with the proviso that the compound of Formula (I) is not isoandrographolide.

In various embodiments, the compounds of Formula (I) or Formula (II), and their pharmaceutically acceptable compositions, are direct or indirect inhibitors or direct or indirect modulators of one or more of the following therapeutic targets: neutrophil migration, cathepsin D (Cath D), surfactant protein C (SPC), glutathione (GSH), collagen, type I, alpha 1 (COL1A1), pyruvate carboxylase (PC), 90 kD heat shock protein (Hsp90B), and transforming growth factor-beta (TGF-1), and thus are useful for treating or lessening the severity of the variety of diseases, disorders, or conditions described herein.

In these and other embodiments, because the compounds of Formula (I) or Formula (II), and their pharmaceutically acceptable compositions, are direct or indirect inhibitors or direct or indirect modulators of neutrophil migration, the compounds of Formula (I) or Formula (II), and their pharmaceutically acceptable compositions, may be suitable for treating and/or preventing cardiovascular diseases and conditions (e.g., atherosclerosis, acute coronary ischaemia, heart failure, peripheral artery disease, aortic aneurysm, arrythmia, and acute myocardial infarction (AMI)); endocrine diseases and conditions (e.g., CKD, tubulointerstitial fibrosis (TIF), gout, and diabetes); neurological diseases and conditions (e.g., Alzheimer's disease); gastrointestinal diseases and conditions (e.g., pancreatitis IBD, and ulcerative colitis); autoimmune diseases and conditions (e.g., lupus erythematosus (SLE), vasculitis, and rheumatoid arthritis (RA)); hematological diseases and conditions (e.g., sickle cell disease (VOC)), pulmonary diseases and conditions (e.g., COPD, IPF, asthma, type 2-low neutrophilic asthma, and cystic fibrosis), dermatological diseases and conditions (e.g., atopic dermatitis, lupus (SLE), psoriasis, dermatomyositis (indiopathic inflammatory myopathy), hidradenitis suppurativa (HS), sweet syndrome, pyoderma gangrenosum (PG), acne, Schnitzler's syndrome, and adult onset Still's disease); or combinations thereof.

As described herein, the compounds of Formula (I) or Formula (II) are provided as inhibitors or modulators of one or more of the following therapeutic targets: neutrophil migration, cathepsin D (Cath D), surfactant protein C (SPC), glutathione (GSH), collagen, type I, alpha 1 (COL1A1), pyruvate carboxylase (PC), 90 kD heat shock protein (Hsp90β), and transforming growth factor-beta (TGF-1). In relation to, or independent of, this inhibitory or modulatory activity, the present compounds are useful for the treatment of diseases, disorders, and conditions including, but not limited to fibrosis and atopic dermatitis. Accordingly, in another aspect of the disclosure, pharmaceutically acceptable compositions are provided, wherein these compositions comprise any of the compounds of Formula (I) or Formula (II) as described herein, and optionally comprise a pharmaceutically acceptable carrier, adjuvant or vehicle. In certain embodiments, these compositions optionally further comprise one or more additional therapeutic agents.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1F depict a series of bar graphs illustrating the reduction in fibrotic symptoms in a bleomycin-induced pulmonary fibrosis model upon treatment with either compound 3 or various controls. For each bar graph, the bars, from left to right, denote a normal control, bleomycin (control), pirfenidone (control) at 100 mpk, compound 3 at 1 mpk, compound 3 at 3 mpk, compound 3 at 10 mpk, and compound 3 at 30 mpk.

FIGS. 2A-2D depict a series of bar graphs illustrating the therapeutic effects of compound 3 relative to various controls. For each bar graph, the bars, from left to right, denote a normal control, bleomycin (control), pirfenidone (control) at 100 mpk, compound 3 at 10 mpk (P), compound 3 at 30 mpk (P), compound 3 at 100 mpk (P), and compound 3 at 100 mpk (T).

FIGS. 3A-3G depict a series of histological images illustrating the therapeutic effects of compound 3 relative to various controls.

FIGS. 4A-4G depict a series of bar graphs comparing the levels of TNF-α, IL-6, IL-36γ, total cell count (BALF), neutrophils (BALF), neutrophils (blood), and eosinophils (BALF), MIP-2 upon treatment with either compound 3 or various controls. For each bar graph, the bars, from left to right, denote a native control, treatment-free control (LPS), dexamethasone (comparative), and Compound 3 at 30 mpk.

FIGS. 5A-5G depict a series of bar graphs illustrating the effects of compound 3 on inflammatory cytokines in a lipopolysaccharide (LPS)-induced acute lung injury model, relative to various controls. For each bar graph, the bars, from left to right, denote a normal control, bleomycin (control), pirfenidone (control) at 100 mpk, compound 3 at 3 mpk, compound 3 at 10 mpk, and compound 3 at 30 mpk.

FIGS. 6A-6D depicts a series of bar graphs illustrating the effects of compound 3 on biomarkers associated with inflammation and fibrosis in human precision-cut lung slices, relative to various controls. For each bar graph, the bars, from left to right, denote a normal control, nintedanib (control) at 2.5 μM, pirfenidone (control) at 2.5 mM, compound 3 at 5 μM, and compound 3 at 50 μM.

FIGS. 7A-7B depict line graphs showing the effect of compound 3 on IL-8 induced chemotaxis, as compared to Navarixin.

FIGS. 7C-7D depict line graphs showing the effect of compound 3 on fMLP induced chemotaxis, as compared to HCH6-1

FIGS. 8A-8D depict a line graph and a series of bar graphs showing the effect of compound 1 on disease activity and parameters in a DSS mouse model of IBD.

FIGS. 9A-9B depict a series of bar graphs showing the effect of compound 1 on peritoneal thickness and α-SMA in a mouse model of peritoneal fibrosis. For each bar graph, the bars, from left to right, denote a sham control, 0.1% CG+vehicle, SB525334 30 mpk, compound 1 at 10 mpk, compound 1 at 30 mpk, and compound 1 at 100 mpk.

FIGS. 10A-10B depict line graphs showing the effect of compound 1 on ear thickness and PASI scores in an IMQ psoriasis model.

FIGS. 11A-11C depict line graphs showing the effect of compound 1 on body weight, fold back thickness, and dermatitis severity score in a MC903-induced atopic dermatitis mouse model.

FIG. 12A depicts the dermatitis severity scores of Compound 2 in an MC-903 animal model.

FIG. 12B is a pictorial representation of mouse back skin post MC-903 application in different mice groups.

FIG. 12C depicts compound 2 robustly reduces epidermal thickness, a key sign of AD lesions, in the MC903-induced model of Atopic Dermatitis along with neutrophil infiltration.

FIG. 12D is a graphical representation of epidermal thickness and tissue Lipocalin-2 (NGAL).

FIG. 12E shows that compound 2 reduced dose-dependent immune cell infiltration as measured by histopathology concurrent with observed efficacy.

FIG. 12F represents that compound 2 significantly reduces NGAL levels demonstrating a clear mechanistic link.

FIG. 12G depicts compound 2 reduces early barrier alarmins in skin that are elevated in skin lesions of AD patients.

FIG. 12H depicts compound 2 impacts tissue cytokine expression directly involved in the progression of atopic dermatitis pathophysiology.

FIG. 12I depicts compound 2 decreases IL-31 expression, a cytokine directly linked to pruritus in AD.

FIG. 13A depicts compound 2 showed dose dependent decrease in dermatitis severity scores when compared with DNFB vehicle control.

FIG. 13B depicts compound 2 reduces immune cell infiltration and ameliorates DNFB-induced histopathology.

FIG. 13C depicts compound 2 significantly reduces epidermal thickness and histopathology score.

FIG. 13D depicts compound 2 impacts IL-4 expression, a key cytokine directly involved in the pathogenic cascade of atopic dermatitis.

FIG. 13E depicts compound 2 reduces itch comparably to current first line therapy for AD.

FIG. 14 is a graphical representation of non-limiting embodiments of an exemplary compound 2 wherein compound 2 significantly reduces dermatitis erythema & dermatitis scaling scores in the MC-903 model of atopic dermatitis.

FIG. 15 is a graphical representation of non-limiting embodiments of an exemplary compound 2 wherein compound 2 significantly reduces dermatitis severity scores on-par with oral JAK-i in the MC903 AD model.

FIG. 16 is a graphical representation of non-limiting embodiments of an exemplary compound 2 wherein compound 2 significantly reduces dermatitis severity scores as low as 0.1 mg/kg in the MC903 model of AD.

FIG. 17 is a graphical representation of an alternative synthesis route for forming Compound 3.

### DETAILED DESCRIPTION

Provided herein are compounds, compositions, and methods for treating, reducing, or ameliorating a variety of diseases, disorders, or conditions in a subject in need thereof. In certain embodiments, the present disclosure relates to compounds, such as the compounds of Formula (I) or Formula (II), that act as neutrophil migration modulators and their use in treating disease or conditions, such as fibrosis.

Furthermore, as described above, the compounds of Formula (I) or Formula (II) are provided as inhibitors or modulators of one or more of the following therapeutic targets: neutrophil migration, cathepsin D (Cath D), surfactant protein C (SPC), glutathione (GSH), collagen, type I, alpha 1 (COL1A1), pyruvate carboxylase (PC), 90 kD heat shock protein (Hsp90B), and transforming growth factor-beta (TGF-β1). In relation to, or independent of, this inhibitory or modulatory activity, the present compounds are useful for the treatment of diseases, disorders, and conditions including, but not limited to, fibrosis.

In these and other embodiments, because the compounds of Formula (I) or Formula (II), and their pharmaceutically acceptable compositions, are direct or indirect inhibitors or direct or indirect modulators of neutrophil migration, the compounds of Formula (I) or Formula (II), and their pharmaceutically acceptable compositions, may be suitable for treating and/or preventing cardiovascular diseases and conditions (e.g., atherosclerosis, acute coronary ischaemia, heart failure, peripheral artery disease, aortic aneurysm, arrythmia, and acute myocardial infarction (AMI)); endocrine diseases and conditions (e.g., CKD, tubulointerstitial fibrosis (TIF), gout, and diabetes); neurological diseases and conditions (e.g., Alzheimer's disease); gastrointestinal diseases and conditions (e.g., pancreatitis IBD); autoimmune diseases and conditions (e.g., lupus erythematosus (SLE), vasculitis, and rheumatoid arthritis (RA)); hematological diseases and conditions (e.g., sickle cell disease (VOC)), pulmonary diseases and conditions (e.g., COPD, IPF, asthma, type 2-low neutrophilic asthma, and cystic fibrosis), dermatological diseases and conditions (e.g., atopic dermatitis, lupus (SLE), psoriasis, dermatomyositis (indiopathic inflammatory myopathy), hidradenitis suppurativa (HS), sweet syndrome, pyoderma gangrenosum (PG), acne, Schnitzler's syndrome, and adult onset Still's disease); or combinations thereof.

The present disclosure also includes pharmaceutical compositions containing any of the compounds provided herein and a pharmaceutically acceptable carrier. The present disclosure also includes kits containing the pharmaceutical compositions. The present disclosure further includes methods of treating a pathological state in a mammal through the administration of any compounds provided herein.

### Compounds

A compound of Formula (I) or Formula (II) is provided herein, as follows:

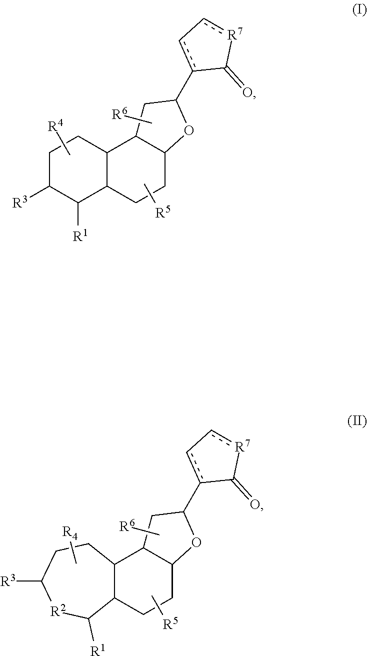

or pharmaceutically acceptable salts thereof, wherein:
R¹ is a alkyl, —CN, —NH₂, —OH, —CH₂OH, —CHO, —CH₂R⁸, —COOH, —CONH₂, —COR⁸, —N₃, —SH—S(alkyl), a halogen atom, aryl, heteroaryl, or linked to R³ to form a 3- to 10-membered cyclic, heterocyclic, or heteroaromatic group, wherein the alkyl, the aryl, the heteroaryl, and the cyclic or heterocyclic group are unsubstituted or substituted with one or more oxo or R⁸; R² is oxygen or NR⁹;
R³ is a hydroxyl, ═O, ═N—OH, halogen, —SH, —S(alkyl), —NR⁹R¹⁰, —COOH, —COOR⁹, —OCOR⁹, —CONH₂, —CN, —N₃, haloalkyl, or alkoxy;
R⁴, R⁵, and R⁶ are each, independently, hydrogen or a unsubstituted or substituted alkyl; R⁷ is oxygen, nitrogen, or NR⁹;
each R⁸ is, independently, a hydroxyl, a halogen, —SH, —S(alkyl), —NR⁹R¹⁰, —COOH, —COOR⁹, —OCOR⁹, —CONH₂, —COR⁹, —COHR⁹R¹⁰, —CN, —N₃, alkyl, haloalkyl, alkenyl, alkynyl, alkoxy, aryl, aralkyl, or heterocyclyl; and
R⁹ and R¹⁰ are each, independently, hydrogen, cycloalkyl, or C₁-C₅ unsubstituted or substituted alkyl;
with the proviso that the compound of Formula (I) is not isoandrographolide.

In some embodiments,
R¹ is a C₁-C₆ alkyl, —CN, —NH₂, —OH, —CH₂OH, —CHO, —CH₂R⁸, —COOH, —CONH₂, —COR⁸, —N₃, —SH—SMe, a halogen atom, aryl, heteroaryl, or linked to R³ to form a 3- to 10-membered cyclic, heterocyclic, or heteroaromatic group, wherein the C₁-C₆ alkyl, the aryl, the heteroaryl, and the cyclic or heterocyclic group are unsubstituted or substituted with one or more oxo or R⁸;
R² is oxygen or NR⁹;
R³ is a hydroxyl, ═O, ═N—OH, halogen, —SH, —SMe, —NR⁹R¹⁰, —COOH, —COOR⁹, —OCOR⁹, —CONH₂, —CN, —N₃, C₁-C₅ haloalkyl, or C₁-C₅ alkoxy;
R⁴, R⁵, and R⁶ are each, independently, hydrogen or a C₁-C₅ unsubstituted or substituted alkyl;
R⁷ is oxygen, nitrogen, or NR⁹;
each R⁸ is, independently, a hydroxyl, a halogen, —SH, —SMe, —NR⁹R¹⁰, —COOH, —COOR⁹, —OCOR⁹, —CONH₂, —COR⁹, —COHR⁹R¹⁰, —CN, —N₃, C₁-C₅ alkyl, C₁-C₅ haloalkyl, C₂-C₅ alkenyl, C₂-C₅ alkynyl, C₁-C₅alkoxy, aryl, aralkyl, or heterocyclyl; and
R⁹ and R¹⁰ are each, independently, hydrogen, C₃-C₈ cycloalkyl, or C₁-C₅ alkyl, wherein each C₁-C₅ alkyl is optionally substituted by phenyl, and wherein each phenyl is optionally substituted by one or more groups selected from C₁-C₅ alkyl and halogen;

with the proviso that the compound of Formula (I) is not isoandrographolide.

In some embodiments,
R¹ is a alkyl, —CN, —NH₂, —OH, —CH₂OH, —CHO, —CH₂R⁸, —COOH, —CONH₂, —COR⁸, —N₃, —COR⁸, —SH—S(alkyl), halogen, aryl, heteroaryl, or linked to R³ to form a 3- to 10-membered cyclic or heterocyclic group, wherein the alkyl, the aryl, the heteroaryl, and the cyclic or heterocyclic group are unsubstituted or substituted with one or more R⁸;
R² is oxygen or NR⁹;
R³ is a hydroxyl, ═O, halogen, —SH, —S(alkyl), —NR⁹R¹⁰, —COOH, —COOR⁹, —CONH₂, —CN, —N₃, haloalkyl, or alkoxy;
R⁴, R⁵, and R⁶ are each, independently, hydrogen or a unsubstituted or substituted alkyl;
R⁷ is oxygen, nitrogen, or NR⁹;
each R⁸ is, independently, hydroxyl, halogen, —SH, —S(alkyl), —NR⁹R¹⁰, —COOH, —COOR⁹, —CONH₂, —COR⁹, —COHR⁹R¹⁰, —CN, —N₃, alkyl, haloalkyl, alkenyl, alkynyl, alkoxy, aryl, or aralkyl; and
R⁹ and R¹⁰ are each, independently, hydrogen or am unsubstituted or substituted alkyl; with the proviso that the compound of Formula (I) is not isoandrographolide.

In some embodiments,
R¹ is a C₁-C₆ alkyl, —CN, —NH₂, —OH, —CH₂OH, —CHO, —CH₂R⁸, —COOH, —CONH₂, —COR⁸, —N₃, —COR⁸, —SH—SMe, a halogen atom, aryl, heteroaryl, or linked to R³ to form a 3- to 10-membered cyclic or heterocyclic group, wherein the C₁-C₆ alkyl, the aryl, the heteroaryl, and the cyclic or heterocyclic group are unsubstituted or substituted with one or more R⁸;
R² is oxygen or NR⁹;
R³ is a hydroxyl, —O, halogen, —SH, —SMe, —NR⁹R¹⁰, —COOH, —COOR⁹, —CONH₂, —CN, —N₃, C₁-C₅ haloalkyl, or C₁-C₅ alkoxy;
R⁴, R⁵, and R⁶ are each, independently, hydrogen or a C₁-C₅ unsubstituted or substituted alkyl;
R⁷ is oxygen, nitrogen, or NR⁹;
each R⁸ is, independently, a hydroxyl, a halogen, —SH, —SMe, —NR⁹R¹⁰, —COOH, —COOR⁹, —CONH₂, —COR⁹, —COHR⁹R¹⁰, —CN, —N₃, C₁-C₅alkyl, C₁-C₅ haloalkyl, C₂-C₅ alkenyl, C₂-C₅ alkynyl, C₁-C₅alkoxy, aryl, or aralkyl; and
R⁹ and R¹⁰ are each, independently, hydrogen or a C₁-C₅ unsubstituted or substituted alkyl;
with the proviso that the compound of Formula (I) is not isoandrographolide.

For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated by reference.

As described herein, compounds of the disclosure can optionally be substituted with one or more substituents, such as are illustrated generally above, or as exemplified by particular classes, subclasses, and species of the disclosure. As one of ordinary skill in the art will recognize, combinations of substituents envisioned by this disclosure are those combinations that result in the formation of stable or chemically feasible compounds. The term “stable,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40 °C or less, in the absence of moisture or other chemically reactive conditions, for at least a week, optionally at least 4 weeks, optionally at least 8 weeks, or optionally at least 12 weeks.

The phrase “optionally substituted” may be used interchangeably with the phrase “substituted or unsubstituted.” In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this disclosure are those combinations that result in the formation of stable or chemically feasible compounds.

The phrase “up to,” as used herein, refers to zero or any integer number that is equal or less than the number following the phrase. For example, “up to 4” means any one of 0, 1, 2, 3, and 4.

The term “aliphatic,” “aliphatic group” or “alkyl” as used herein, means a straight-chain (i.e. unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. Suitable aliphatic groups include, but are not limited to, linear or branched, substituted or unsubstituted alkyl, alkenyl, alkynyl groups.

The terms “cycloaliphatic” or “cycloalkyl” mean a monocyclic hydrocarbon ring, or a polycyclic hydrocarbon ring system that is completely saturated or that contains one or more units of unsaturation, but which is not aromatic and has a single point of attachment to the rest of the molecule. The term “polycyclic ring system,” as used herein, includes bicyclic and tricyclic 4- to 12-membered structures that form at least two rings, wherein the two rings have at least one atom in common (e.g., 2 atoms in common) including fused, bridged, or spirocyclic ring systems.

The term “halogen” or “halo” as used herein, means F, Cl, Br or I. Unless otherwise specified, the term “heterocycle,” “heterocyclyl,” “heterocycloaliphatic,” “heterocycloalkyl,” or “heterocyclic” as used herein means non-aromatic, monocyclic, bicyclic, or tricyclic ring systems in which one or more ring atoms in one or more ring members is an independently selected heteroatom. Heterocyclic ring can be saturated or can contain one or more unsaturated bonds. In some embodiments, the “heterocycle,” “heterocyclyl,” “heterocycloaliphatic,” “heterocycloalkyl,” or “heterocyclic” group has three to fourteen ring members in which one or more ring members is a heteroatom independently selected from oxygen, sulfur, nitrogen, or phosphorus, and each ring in the ring system contains 3 to 7 ring members.

The term “heteroatom” means oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation but is not aromatic.

The term “alkoxy,” or “thioalkyl,” as used herein, refers to an alkyl group, as previously defined, attached to the principal carbon chain through an oxygen (“alkoxy”) or sulfur (“thioalkyl”) atom.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring carbon atoms, wherein at least one ring in the system is aromatic and wherein each ring in the system contains 3 to 7 ring carbon atoms. The term “aryl” may be used interchangeably with the term “aryl ring.”

The term “heteroaryl,” used alone or as part of a larger moiety as in “heteroaralkyl” or “heteroarylalkoxy,” refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic.”

“D” and “d” both refer to deuterium.

A dashed bond in a structure denotes a bond that may be present or absent. For example, [symbol] denotes a bond that is a single bond or a double bond.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of Formula (I) of the disclosure are within the scope of the disclosure. Thus, included within the scope of the disclosure are tautomers of compounds of Formula (I). The structures also include zwitterionic forms of the compounds or salts of Formula (I) where appropriate.

Additionally, unless otherwise stated, structures depicted herein are also meant to include compounds that differ only in the presence of one or more isotopically enriched or isotopically-labeled atoms. The isotopically-labeled compounds may have one or more atoms replaced by an atom having an atomic mass or mass number usually found in nature. Examples of isotopes present in compounds of Formula (I) include isotopes of hydrogen, carbon, nitrogen, oxygen, phosphorus, fluorine and chlorine, such as, but not limited to, 2H, 3H, 13C, 14C, 15N, 18O, 17O, 35S and 18F. Certain isotopically-labeled compounds of Formula (I), in addition to being useful as therapeutic agents, are also useful in drug and/or substrate tissue distribution assays, as analytical tools or as probes in other biological assays. In one aspect of the present disclosure, tritiated (e.g., 3H) and carbon-14 (e.g., 14C) isotopes are useful given their ease of detectability. In another aspect of the present disclosure, replacement of one or more hydrogen atoms with heavier isotopes such as deuterium, (e.g., 2H) can afford certain therapeutic advantages.

In certain embodiments, the compound of Formula (I) is Formula (Ia) and the compound of Formula (II) is Formula (IIa), as follows:

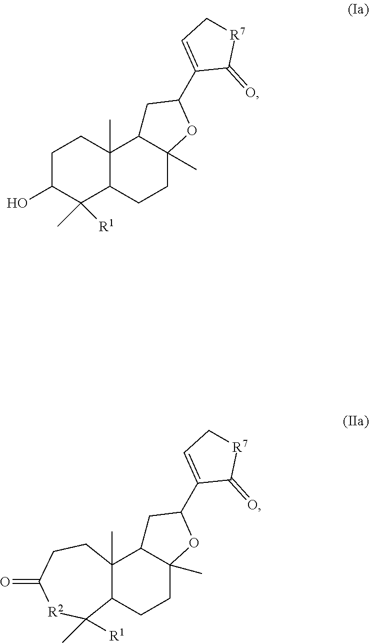

or pharmaceutically acceptable salts thereof, wherein:
R¹ is a C₁-C₆ alkyl, —CN, —NH₂, —OH, —CH₂OH, CHO, —CH₂R⁸, —COOH, —CONH₂, —COR⁸, —N₃, —COR⁸, —SH—SMe, a halogen atom, aryl, or heteroaryl, wherein the C₁-C₆ alkyl, the aryl, and the heteroaryl are unsubstituted or substituted with one or more R⁸;
R² is oxygen or NR⁹;
R⁷ is oxygen, nitrogen, or NR⁹;
each R⁸ is, independently, a hydroxyl, a halogen, —SH, —SMe, —NR⁹R¹⁰, —COOH, —COOR⁹, —CONH₂, —COR⁹, —COHR⁹R¹⁰, —CN, —N₃, C₁-C₅ alkyl, C₁-C₅ haloalkyl, C₂-C₅ alkenyl, C₂-C₅ alkynyl, C₁-C₅ alkoxy, aryl, or aralkyl; and
R⁹ and R¹⁰ are each, independently, hydrogen or a C₁-C₅ unsubstituted or substituted alkyl;
with the proviso that the compound of Formula (Ia) is not isoandrographolide.

In some embodiments, the compound of Formula (Ia) is Formula (Iaa) and the compound of Formula (IIa) is Formula (IIaa), as follows:

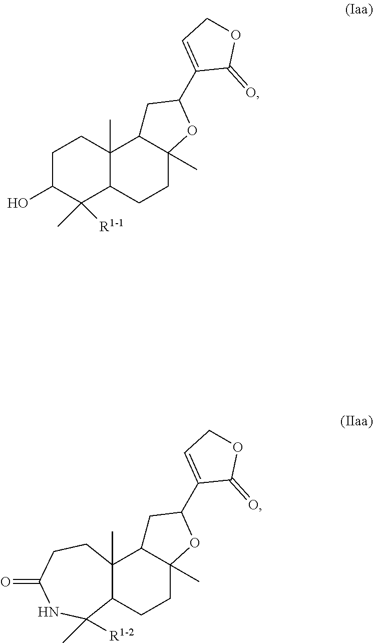

or pharmaceutically acceptable salts thereof, wherein:
R¹⁻¹ is-CN, —NH₂, —OH, —COOH, CHO, —CH₂R⁸, —CONH₂, —COR⁸, —N₃, —COR⁸, —SH, —SMe, a halogen atom, aryl, or heteroaryl, wherein the aryl and the heteroaryl are unsubstituted or substituted with one or more R⁸;
R¹⁻² is a C₁-C₆ alkyl, —CN, —NH₂, —OH, —CH₂OH, —COOH, CHO, —CH₂R⁸, —CONH₂, —COR⁸, —N₃, —COR⁸, —SH, —SMe, a halogen atom, aryl, or heteroaryl, wherein the C₁-C₆ alkyl, the aryl, and the heteroaryl are unsubstituted or substituted with one or more R⁸;
each R⁸ is, independently, a hydroxyl, a halogen, —SH, —SMe, —NR⁹R¹⁰, —COOH, —COOR⁹, —CONH₂, —COR⁹, —COHR⁹R¹⁰, —CN, —N₃, C₁-C₅ alkyl, C₁-C₅ haloalkyl, C₂-C₅ alkenyl, C₂-C₅ alkynyl, C₁-C₅ alkoxy, aryl, or aralkyl; and
R⁹ and R¹⁰ are each, independently, hydrogen or a C₁-C₅ unsubstituted or substituted alkyl.

In certain embodiments, the compound is of Formula (Iaa). In these and other embodiments, R¹⁻¹ is-CN. In exemplary embodiments, the compound of Formula (Iaa) is:

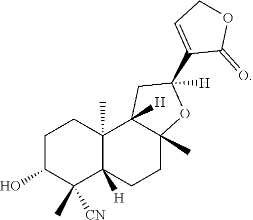

In certain embodiments, the compound is of Formula (IIaa). In some embodiments, the compound of Formula (IIaa) is selected from the group of:

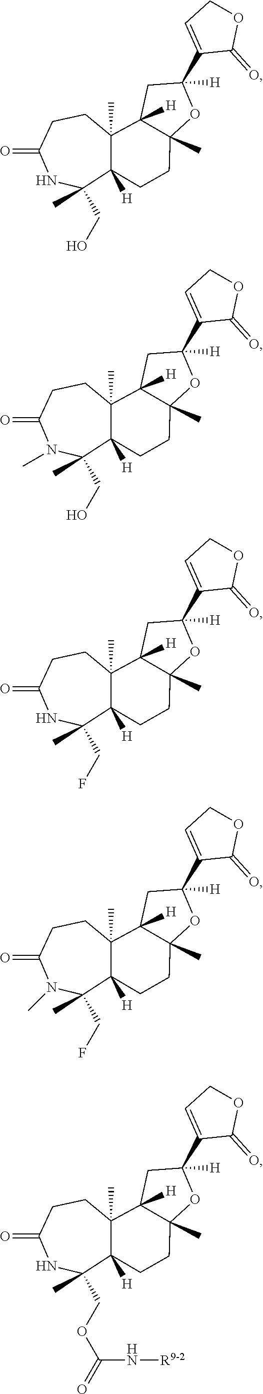

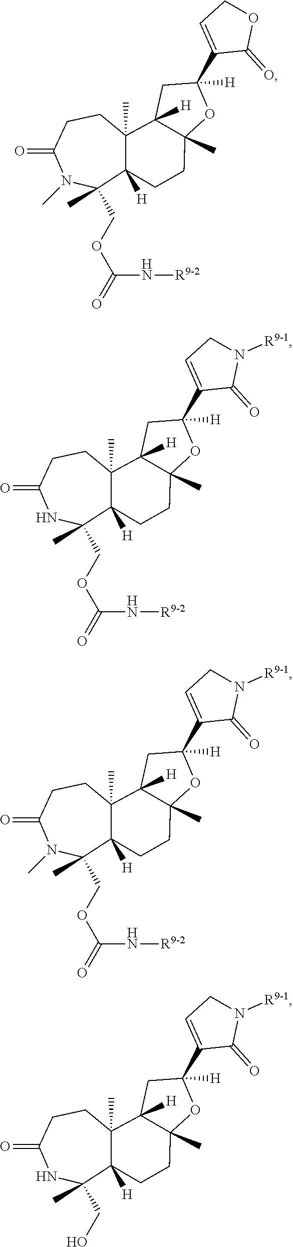

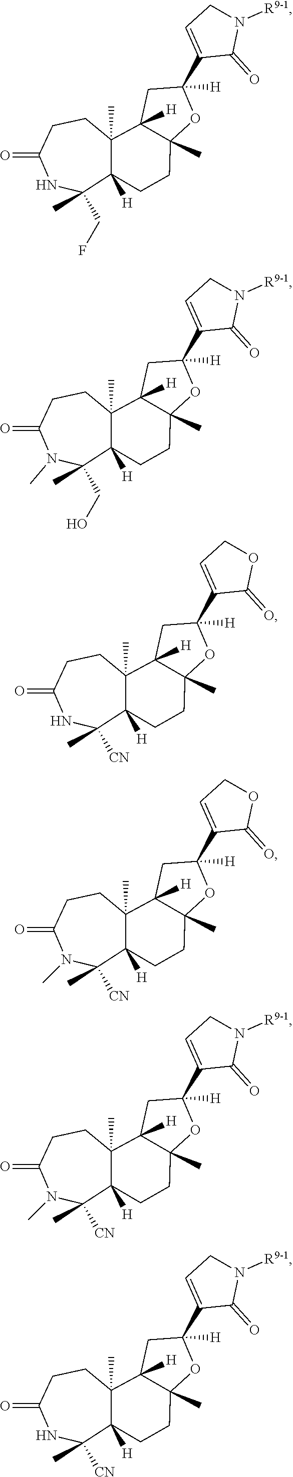

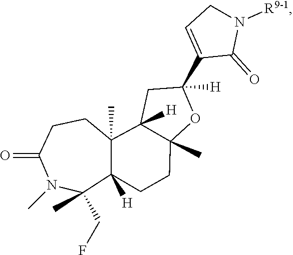

or a pharmaceutically acceptable salt or stereoisomer thereof, wherein:
R⁹⁻¹ is hydrogen or methyl; and
R⁹⁻² is methyl, ethyl, propyl, isopropyl, cyclopropyl, butyl, secbutyl, or cyclobutyl.

In these and other embodiments, R¹⁻² is —CH₂OH or —CN. In some exemplary embodiments, the compound is:

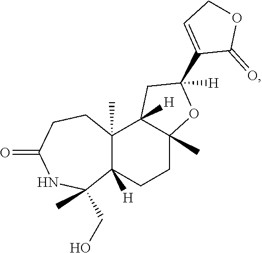

or a pharmaceutically acceptable salt or stereoisomer thereof. In other exemplary embodiments, the compound is:

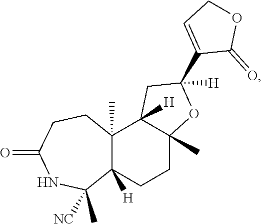

or a pharmaceutically acceptable salt or stereoisomer thereof.

In various embodiments, the compound of Formula (I) or Formula (II) are provided as inhibitors or modulators of one or more of the following therapeutic targets: neutrophil migration, cathepsin D (Cath D), surfactant protein C (SPC), glutathione (GSH), collagen, type I, alpha 1 (COL1A1), pyruvate carboxylase (PC), 90 kD heat shock protein (Hsp90β), and transforming growth factor-beta (TGF-β1). In relation to, or independent of, this inhibitory or modulatory activity, the present compounds are useful for the treatment of diseases, disorders, and conditions including, but not limited to, fibrosis.

### Pharmaceutically Acceptable Salts and Compositions

In another aspect of the disclosure, pharmaceutically acceptable compositions are provided, wherein these compositions comprise the compound of Formula (I) or Formula (II) as described herein, and optionally comprise a pharmaceutically acceptable carrier, adjuvant or vehicle. In certain embodiments, these compositions optionally further comprise one or more additional therapeutic agents.

It will also be appreciated that certain compounds of this disclosure can exist in free form for treatment, or where appropriate, as a pharmaceutically acceptable derivative thereof.

According to the disclosure, a pharmaceutically acceptable derivative includes, but is not limited to, pharmaceutically acceptable salts, esters, salts of such esters, or any other adduct or derivative which upon administration to a subject in need is capable of providing, directly or indirectly, a compound as otherwise described herein, or a metabolite or residue thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this disclosure that, upon administration to a recipient, is capable of providing, either directly or indirectly, a compound of this disclosure or an inhibitory active metabolite or residue thereof. As used herein, the term “inhibitory active metabolite or residue thereof” means that a metabolite or residue thereof is also an inhibitor or modulator of one or more of the therapeutic targets.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference in its entirety. Pharmaceutically acceptable salts of the compound of Formula (I) or Formula (II) of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄ alkyl)₄ salts. This disclosure also envisions the quaternization of any basic nitrogen-containing groups of the compound of Formula (I) or Formula (II) disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, the pharmaceutically acceptable compositions of the disclosure additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compound of Formula (I) or Formula (II) of the disclosure, such as by producing any undesirable biological effect or otherwise interacting in a deleterious manner with any other component(s) of the pharmaceutically acceptable composition, its use is contemplated to be within the scope of this disclosure. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the disclosure features a pharmaceutical composition comprising the compound of the disclosure and a pharmaceutically acceptable carrier.

In another aspect, the disclosure features a pharmaceutical composition comprising a therapeutically effective amount of the compound of Formula (I) or Formula (II), or a pharmaceutically acceptable salt thereof, and one or more pharmaceutically acceptable carriers or vehicles.

The compound of Formula (I) or Formula (II), or a pharmaceutically acceptable salt thereof used in the compositions administered may be obtained from any commercially available sources, or any methods or techniques known in the art. For example, in some variations, the compound of Formula (I) or Formula (II) may be isolated from a natural source. In other variations, the compound of Formula (I) or Formula (II) may be synthesized according to any methods known in the art.

In various embodiments, the composition includes the compound of Formula (I) or Formula (II), or a pharmaceutically acceptable salt thereof, and optionally consists essentially of the compound of Formula (I) or Formula (II), or a pharmaceutically acceptable salt thereof. As used herein, the phrase “consisting essentially of” generally encompasses the specifically recited elements/components for a particular embodiment. Further, the phrase “consisting essentially of” generally encompasses and allows for the presence of additional or optional elements/components that do not materially impact the basic and/or novel characteristics of that particular embodiment. In certain embodiments, “consisting essentially of” allows for the presence of ≤10, ≤5, or ≤1, weight percent (wt. %) of additional or optional components based on the total weight of the composition.

In various embodiments, the composition includes one or more pharmaceutically acceptable additives that are inactive ingredients. Examples of inactive ingredients include, but are not limited to, excipients, such as diluents and binders, granulating agents, glidants (or flow aids), fillers, lubricants, preservatives, stabilizers, coatings, disintegrants, fragrances, pigments, preservatives, solvents (e.g., alcohols), and combinations thereof. If utilized to form the composition, the inactive ingredient(s) can be used in various amounts and combined with the compound of Formula (I) or Formula (II), or a pharmaceutically acceptable salt thereof, to form a composition that is suitable for topical application to human or animal skin. It is to be further appreciated that the amounts of actives described herein can be normalized with respect to 100 parts by weight of the composition to account for the presence of inactive ingredients (if utilized).

Optionally, the composition may include one or more additional components such as additives. Suitable additives include those understood in the art, including but not limited to, moisturizers, emollients, emulsifiers, surfactants, oils, extracts, skin protectants, disinfectants, antiseptics, drugs and drug substances, analgesic compounds, anti-neuralgic compound, anti-oxidants, blood circulation promoters, antidepressant compounds, anti-anxiety compounds, anti-stress compounds, sunscreens, insect repellants, preservatives, exfoliants, fragrances, colors, fillers, solvents, vehicles, carriers, other types of additives known to those of skill in the art, and combinations thereof. Such additives may be utilized alone or in combination. In general, the optional additives may be of any type used in pharmaceuticals, nutraceuticals, personal care products and cosmetic products.

Examples of such carrier components are oils, fats, waxes, surfactants, humectants, thickening agents, antioxidants, viscosity stabilizers, chelating agents, buffers, preservatives, perfumes, dyestuffs, lower alkanols, and the like. If desired, further ingredients may be incorporated in the compositions, e.g., anti-inflammatory agents, antibacterials, antifungals, disinfectants, vitamins, sunscreens, antibiotics, skin bleaching agents, healing enhancers/fibroblast proliferation compounds, neuromuscular blocking agents, sunscreens, or other anti-acne agents.

Examples of oils as a carrier agent comprises fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristatc, isopropyl palmitate and butyl stearate. As examples of surfactants as carrier agents, there may be cited anionic surfactants such as sodium stearate, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrocloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolarnide, polyoxypropylene glycol (such as the materials sold under the trademark “Pluronic”), polyoxyethylene castor oil, and polyoxyethylene lanolin. Examples of humectants as carrier agents include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and, sodium carboxymethyl cellulose. Examples of antioxidants comprise butylated hydroxytoluene, butylated hydroxyanisole, propyl gallate, citric acid, ethoxyquin, alpha lipoic acid, vitamin C, vitamin E, co-enzyme Q-10, and idebenone; botanical anti-oxidants include carotenoids such as lycopene; flavonoids such as silymarin (milk thistle), silybin, silydianin, silychristine; soybeans (isoflavins), grape seed extract; polyphenols such as green tea extract, rosmarinic acid (rosemary), hypercin (Saint John's wort), oleuropein (olive leaf), curcurmin (tumeric root), tetrahydrocurcumin, and pycogenol (marine bark pine). Examples of anti-inflammatory agents include anti-inflammatory botanicals such as allantoin, aloe vera, Ginkgo biloba, green tea (also considered an antioxidant). Examples of skin bleaching agents are hydroquinone or kojic acid. Examples of healing enhancers/fibroblast proliferation compounds include copper peptides or palmitoyl-pentapetide (pal-KTTKS). Examples of neuromuscular blocking agents such as acetyl hexapeptide 3 (argircline) or dimethylaminoethanol. Examples of chelating agents include disodium edetate and ethanehydroxy diphosphate. Examples of buffers as carrier agents comprise citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid.

It is to be appreciated that certain components or additives may be classified under different terms of art and just because a component or additive is classified under such a term does not mean that they are limited to that function. If utilized, the additive or additives may be present in the composition in various amounts. Additional ingredients for optional use in the composition are described in U.S. Pat. No. 5,747,006 to Dornoff et al., U.S. Pat. Nos. 5,980,904, 6,994,874, 7,060,304, 7,247,321, and 7,364,759 to Leverett et al., and U.S. Publication No. 2017/0252293 to Brumbaugh et al., the disclosures of which are hereby incorporated by reference in their entirety.

The composition can be prepared using various methods. For example, actives of the composition, and optionally one or more inactives, can be mixed or blended and compressed or compounded utilizing various techniques understood in the art. The composition of this disclosure is not limited to a particular order of manufacturing steps or method of manufacture.

The composition can be in various forms. Examples of suitable forms include solids, gels and liquids. For example, the composition can be formulated for application as a gel, cream, lotion, pomade, mousse, powder, or foam for application to the subject's skin. In another example, the composition can be formulated for spraying onto a subject's skin. The composition can be formulated to be sprayed as either an aerosol spray or pump spray. In still another example, the composition can be formulated for application using a pre-moistened towelette. In another example, the composition can be formulated as a solid that is rubbed onto the subject's skin. In another example, the composition is formulated for delivery through a patch that is adhered to the subject's skin.

The composition may comprise at least about 0.375% w/w, at least about 0.75% w/w, at least about 1% w/w, at least about 1.5% w/w, at least about 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, at least 15% w/w, at least 16% w/w, at least 17% w/w, at least 18% w/w, at least 19% w/w, at least 20% w/w, or even more of the compound, or a pharmaceutically acceptable salt thereof, of Formula (I). In certain variations, the composition comprises about 0.375%, about 0.75%, about 1%, about 1.5%, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, or even more of the compound of Formula (I), or a pharmaceutically acceptable salt thereof.

In certain variations, the composition comprises between about 0.1% and about 20% w/w, between about 0.1% and about 15% w/w, between about 0.1% and about 5% w/w, between about 0.1% and about 3% w/w, between about 0.1% and about 1.5% w/w, between about 0.1% and about 1% w/w, between about 0.1% and about 0.75% w/w, between about 0.1% and about 0.375% w/w, between about 0.375% and about 5% w/w, between about 0.375% and about 3% w/w, between about 0.375% and about 1.5% w/w, between about 0.375% and about 1% w/w, between about 0.375% and about 0.75% w/w, between about 0.75% and about 5% w/w, between about 0.75% and about 3% w/w, between about 0.75% and about 1.5% w/w, between about 0.75% and about 1% w/w, between about 1% and about 5% w/w, between about 1% and about 3% w/w, between about 1% and about 1.5% w/w, between about 1.5% and about 5% w/w, between about 1.5% and about 3% w/w, between about 3% and about 5% w/w, between about 1% and about 15% w/w, between about 2% and about 10% w/w, between about 4% and about 8% w/w, between about 5% and about 15% w/w, or between about 8% and about 12% w/w of the compound of Formula (I), or a pharmaceutically acceptable salt thereof.

In other embodiments, the compound of Formula (I) or Formula (II), or a pharmaceutically acceptable salt thereof, is present in the composition has a purity of at least about 50% w/w, at least about 60% w/w, at least about 70% w/w, at least about 75% w/w, at least about 80% w/w, at least about 90% w/w, at least about 95% w/w, at least about 96% w/w, at least about 97% w/w, at least about 98% w/w, at least about 99% w/w, at least about 99.9% w/w, at least about 99.99% w/w, or at least about 99.999% w/w; or a purity of about 100% w/w.

In other variations of the foregoing, the composition further comprises one or more additional components, including for example, fragrants, colorants, and/or excipients.

### Uses of Compounds and Pharmaceutically Acceptable Salts and Compositions

These pharmaceutically acceptable compositions are useful for treating or lessening the severity of a variety of diseases, disorders, or conditions, including, but not limited to, fibrosis. In various embodiments, the pharmaceutically acceptable compositions, are inhibitors or modulators of one or more of the following therapeutic targets: neutrophil migration, cathepsin D (Cath D), surfactant protein C (SPC), glutathione (GSH), collagen, type I, alpha 1 (COL1A1), pyruvate carboxylase (PC), 90 kD heat shock protein (Hsp90B), and transforming growth factor-beta (TGF-β1), and thus are useful for treating or lessening the severity of the variety of diseases, disorders, or conditions described herein.

In various embodiments, the terms “inhibits” and “modulates” are used interchangeably to refer to an agent that decreases or suppresses a biological activity, such as to repress an activity of a therapeutic targets, such as a neutrophil migration modulator that includes modulators having any combination of the structural and/or functional properties disclosed herein.

A method of treating, ameliorating, or preventing a neutrophil-related condition or disease, comprising administering a therapeutically effective amount of the compound of Formula (I) or Formula (II) is provided herein. In various embodiments, the condition or disease is selected from the group of cardiovascular condition or disease, an endocrine condition or disease, a neurological condition or disease, a gastrointestinal condition or disease, a dermatological condition or disease, or combinations thereof.

A method of treating, ameliorating, or preventing a neutrophil-related condition or disease, comprising administering a therapeutically effective amount of the pharmaceutical composition according is also provided herein. In various embodiments, the condition or disease is selected from the group of cardiovascular condition or disease, an endocrine condition or disease, a neurological condition or disease, a gastrointestinal condition or disease, a dermatological condition or disease, or combinations thereof.

A pharmaceutical composition comprising the compound of Formula (I) or Formula (II), or a pharmaceutically acceptable salt or stereoisomer thereof, for use in a method of treating, ameliorating, or preventing a neutrophil-related condition or disease is also provided herein. In various embodiments, the condition or disease is selected from the group of cardiovascular condition or disease, an endocrine condition or disease, a neurological condition or disease, a gastrointestinal condition or disease, a dermatological condition or disease, or combinations thereof.

Use of the compound of Formula (I) or Formula (II), or a pharmaceutically acceptable salt or stereoisomer thereof, in the preparation of a medicament for use in treating, ameliorating, or preventing a fibrotic condition or disease is also provided herein. In various embodiments, the condition or disease is selected from the group of cardiovascular condition or disease, an endocrine condition or disease, a neurological condition or disease, a gastrointestinal condition or disease, a dermatological condition or disease, or combinations thereof.

A method of treating, ameliorating, or preventing a fibrotic condition or disease, comprising administering a therapeutically effective amount of the compound of Formula (I) or Formula (II) is provided herein. In various embodiments, the condition or disease is idiopathic pulmonary fibrosis (IPF).

A method of treating, ameliorating, or preventing a fibrotic condition or disease, comprising administering a therapeutically effective amount of the pharmaceutical composition according is also provided herein. In various embodiments, the condition or disease is idiopathic pulmonary fibrosis (IPF).

A pharmaceutical composition comprising the compound of Formula (I) or Formula (II), or a pharmaceutically acceptable salt or stereoisomer thereof, for use in a method of treating, ameliorating, or preventing a fibrotic condition or disease is also provided herein. In various embodiments, the condition or disease is idiopathic pulmonary fibrosis (IPF).

Use of the compound of Formula (I) or Formula (II), or a pharmaceutically acceptable salt or stereoisomer thereof, in the preparation of a medicament for use in treating, ameliorating, or preventing a fibrotic condition or disease is also provided herein. In various embodiments, the condition or disease is idiopathic pulmonary fibrosis (IPF).

A method of treating, ameliorating, or preventing a dermatological condition or disease, comprising administering a therapeutically effective amount of the compound of Formula (I) or Formula (II) is provided herein. In various embodiments, the condition or disease is atopic dermatitis.

A method of treating, ameliorating, or preventing a dermatological condition or disease, comprising administering a therapeutically effective amount of the pharmaceutical composition according is also provided herein. In various embodiments, the condition or disease is atopic dermatitis.

A pharmaceutical composition comprising the compound of Formula (I) or Formula (II), or a pharmaceutically acceptable salt or stereoisomer thereof, for use in a method of treating, ameliorating, or preventing a dermatological condition or disease is also provided herein. In various embodiments, the condition or disease is atopic dermatitis.

Use of the compound of Formula (I) or Formula (II), or a pharmaceutically acceptable salt or stereoisomer thereof, in the preparation of a medicament for use in treating, ameliorating, or preventing a dermatological condition or disease is also provided herein. In various embodiments, the condition or disease is atopic dermatitis.

In some embodiments, “treating” or “treatment” refers to both prophylactic or preventative treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. In these and other embodiments, “treating” or “treatment” refers to obtaining beneficial or desired results, for example clinical results, in a subject, including: (1) alleviating one or more symptoms caused by or associated with a disease, disorder, or condition; (2) reducing the extent of the disease, disorder, or condition; (3) slowing or stopping the development or progression of one or more symptoms caused by or associated with the disease, disorder, or condition (for example, stabilizing the disease, disorder, or condition); and (4) relieving the disease, for example, by causing the regression of one or more clinical symptoms (e.g., ameliorating the disease state, enhancing the effect of another medication, delaying or stopping the progression of the disease, and/or increasing the quality of life).

In certain embodiments, the compound of Formula (I) or Formula (II), or a pharmaceutically acceptable salt thereof, inhibits or modulated one or more of the therapeutic targets with an IC₅₀ less than 50 micromolar (μM), optionally less than 40 μM, optionally less than 30 μM, optionally less than 20 μM, optionally less than 10 μM, or optionally less than 1 μM. Alternatively, the compound of Formula (I), or a pharmaceutically acceptable salt thereof, inhibits or modulated one or more of the therapeutic targets with an IC₅₀ from about 0.01 μM to about 100 μM, optionally from about 0.1 μM to about 75 μM, optionally from about 1 μM to about 50 μM, optionally from about 1 μM to about 25 μM, or optionally from about 1 μM to about 10 μM.

In some embodiments, the method comprises administering to the subject the compound of Formula (I) or Formula (II), or a pharmaceutically acceptable salt thereof, in an excipient to form a composition. The composition may comprise between 10-500 mg, or between 10-800 mg, or between 20-1,000 mg, or between 30-1,200 mg, or between 50-1,500 mg, or between 100-2,000 mg or even more of the compound of Formula (I) or Formula (II), or a pharmaceutically acceptable salt thereof in a single dosage unit. Viewed from a different perspective, at least 20 wt %, or at least 30 wt %, or at least 40 wt %, or at least 50 wt %, or at least 60 wt %, or at least 70 wt %, or at least 80 wt %, or at least 90 wt % of the composition will be the compound of Formula (I), or a pharmaceutically acceptable salt thereof. Consequently, preferred oral single dosage units (or recommended daily uptake) will be between 20-200 mg, or between 40-400 mg, or between 60-600 mg, or between 80-800 mg, or between 100-1,000 mg, or between 200-2,000 mg, and in some cases even higher.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.).

In some variations, the method comprises administering to the subject the compound of Formula (I) or Formula (II), or a pharmaceutically acceptable salt thereof, in a petroleum-based vehicle, such as petroleum jelly to form a composition. The composition comprises at least about 0.375% w/w, at least about 0.75% w/w, at least about 1% w/w, at least about 1.5% w/w, at least about 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, at least 15% w/w, at least 16% w/w, at least 17% w/w, at least 18% w/w, at least 19% w/w, at least 20% w/w, or even more of Formula (I) or a pharmaceutically acceptable salt thereof. In certain variations, the composition comprises about 0.375%, about 0.75%, about 1%, about 1.5%, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, or even more of the compound of Formula (I) or Formula (II), or a pharmaceutically acceptable salt thereof.

In various embodiments, the amount of composition is administered to the subject is based on the amount of the compound of Formula (I) or Formula (II), or a pharmaceutically acceptable salt thereof, in milligrams (mg) per area of skin in centimeters-squared (cm²). The composition may be administered to the subject in an amount of at least 0.01 mg/cm² (mg of the compound per cm² of skin), at least 0.02 mg/cm². at least 0.03 mg/cm². at least 0.04 mg/cm². at least 0.05 mg/cm². at least 0.06 mg/cm². at least 0.07 mg/cm². at least 0.08 mg/cm². at least 0.09 mg/cm². at least 0.1 mg/cm². at least 0.12 mg/cm². at least 0.14 mg/cm². at least 0.16 mg/cm². at least 0.18 mg/cm². at least 0.2 mg/cm². at least 0.22 mg/cm². at least 0.24 mg/cm². at least 0.26 mg/cm². at least 0.28 mg/cm². at least 0.3 mg/cm². at least 0.32 mg/cm². at least 0.34 mg/cm². at least 0.36 mg/cm². at least 0.3 8 mg/cm². at least 0.4 mg/cm², or even more. The composition may be administered to the subject in an amount of between about 0.01 mg/cm² and about 0.4 mg/cm², between about 0.05 mg/cm² and about 0.3 mg/cm², between about 0.1 mg/cm² and about 3 mg/cm², or between about 0.15 mg/cm² and about 0.25 mg/cm².

The composition may be applied as needed, daily, several times per day or in any suitable regimen such that the desired outcome is achieved. In the method of this disclosure, the frequency of administration (e.g., topical application) can depend on several factors, including the severity of the condition or symptoms of the same or the desired level of relief from the symptoms. Generally, a regimen includes application of the composition once or twice daily to include an administration in the morning and/or an administration in the evening. The amount and/or frequency of application of the composition may depend on several factors, including the level of desired results and the specific composition. In some embodiments, the composition is administered once per day. In other embodiments, the composition is administered twice per day. In still other embodiments, the composition it administered three times per day. In yet other embodiments, the composition is administered four times per day. However, it is to be appreciated that the composition may be administered more than four times per day.

In some variations, the composition is administered for at least about 1 week, at least about 2 weeks, at least about 3 weeks, at least about 4 weeks, at least about 8 weeks, at least about 12 weeks, at least about 16 weeks, at least about 24 weeks, at least about 32 weeks, at least about 40 weeks, at least about 48 weeks, or at least about 1 year. In certain variations, the composition is administered for about 1 week, about 2 weeks, about 3 weeks, about 4 weeks, about 8 weeks, about 12 weeks, about 16 weeks, about 24 weeks, about 32 weeks, about 40 weeks, about 48 weeks, or about 1 year. In one variation, the composition is administered for between about 1 week and about 1 year, between about 4 weeks and about 8 weeks, between about 4 weeks and about 12 weeks, between about 4 weeks and about 16 weeks, between about 4 weeks and about 24 weeks, between about 4 weeks and about 32 weeks, between about 4 weeks and about 40 weeks, between about 4 weeks and about 48 weeks, or between about 4 weeks and about 1 year.

The compound of Formula (I) or Formula (II), a pharmaceutically acceptable salt thereof or a pharmaceutical composition thereof, may be administered as described herein. In some embodiments, the composition is topically administered to the subject. In other embodiments, the composition is orally administered.

In another aspect, the disclosure features a method of inhibiting a therapeutic target, such as neutrophil migration, in a subject comprising administering to the subject the compound of Formula (I) or Formula (II), a pharmaceutically acceptable salt thereof or a pharmaceutical composition thereof. In relation to, or independent of, this inhibitory or modulatory activity, the present compounds are useful for the treatment of diseases, disorders, and conditions including, but not limited to a fibrotic disease.

The term “fibrotic” diseases, disorders, or conditions, as used herein, refers to illnesses, disorders, or conditions that are wholly or partially characterized by an excessive production of fibrous material, including an excessive production of fibrotic material within the extracellular matrix, or by the replacement of normal tissue components by an abnormal, excessive accumulation of matrix-associated components, that is, components that are not functional or that are abnormal. Acute and chronic, clinical or subclinical presentations of fibriotic diseases, disorders, or situations are all possible, and they can all be accompanied by clearly visible fibrogenic biology or pathology.

Examples of fibrotic diseases, disorders and conditions include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g., pulmonary fibrosis, glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, myelodysplastic syndrome, myeloproferative syndrome, gynecological cancer, Kaposi's sarcoma, Hansen's disease, collagenous colitis, acute fibrosis, organ specific fibrosis, and the like.

For example, pulmonary fibrosis, pulmonary hypertension, cystic fibrosis, asthma, chronic obstructive pulmonary disease, liver fibrosis, kidney fibrosis, NASH, and similar conditions are examples of organ-specific fibrotic illnesses. Extracellular matrix is deposited abnormally and/or excessively in many fibrotic illnesses, disorders, and situations. Fibrosis can be brought on by surgery, the healing process after a wound, or an underlying condition. It can also be linked to inflammation. Uncontrolled fibrosis can lead to scarring, which is the deterioration of the underlying organ or tissue's architecture.

The compounds of Formula (I) or Formula (II) may be employed to treat or prevent pulmonary fibrosis in some embodiments. The list of lung fibrosis conditions includes pulmonary fibrosis, pulmonary hypertension, chronic obstructive pulmonary disease (COPD), asthma, silicosis, asbestosis, cystic fibrosis, familial pulmonary fibrosis, sarcoidosis, carbon pneumoconiosis, hypersensitivity pneumonitides, pulmonary fibrosis brought on by inhaling inorganic dust, pulmonary fibrosis brought on by an

A gradual scarring of the lung tissue, excessive buildup of extracellular matrix proteins, and aberrant alveolar structure are the hallmarks of pulmonary fibrosis. The stiffened and swollen tissue makes it harder for the lungs to function properly, which can cause breathing issues including shortness of breath and be fatal. Acute lung injury, viral infection, toxic exposure, radiation exposure, chronic illness, drugs, or an idiopathic (unknown underlying cause) condition can all result in pulmonary fibrosis.

The hallmark signs of idiopathic pulmonary fibrosis are diffuse peripheral lung scarring and tiny bubbles (called bullae) close to the surface of the lung's outer membrane, frequently around the bases of the lungs. The course of idiopathic pulmonary fibrosis is frequently sluggish and unrelenting. Early on, patients frequently lament having a dry, mysterious cough. Next, dyspnea (shortness of breath) develops and gets worse with time as a result of decreasing activity. Shortness of breath eventually limits all activities and can even happen while you're just sitting quietly. In fewer instances, the fibrosis can advance quickly, causing dyspnea and impairment within weeks to months of the disease's beginning. The term Hamman-Rich syndrome has been used to describe this type of pulmonary fibrosis.

A rise in the blood pressure of the lung's vasculature, including the pulmonary artery, pulmonary vein, and/or pulmonary capillaries, is a sign of pulmonary hypertension. The right ventricle of the heart is strained by abnormally high pressure, which causes it to enlarge. Heart failure can develop as a result of the right ventricle losing its strength and capacity to pump adequate blood to the lungs over time. Chronic liver disease and liver cirrhosis, rheumatic diseases like scleroderma or systemic lupus erythematosus (lupus), and lung conditions like tumors, emphysema, chronic obstructive pulmonary disease (COPD), and pulmonary fibrosis can all contribute to pulmonary hypertension. Pulmonary fibrosis may cause the pulmonary vasculature to narrow, which will cause pulmonary hypertension.

A prevalent lung condition known as chronic obstructive pulmonary disease (COPD) is frequently accompanied by emphysema or chronic bronchitis. Cough, mucous buildup, exhaustion, wheezing, and respiratory infections are common symptoms.

Lung conditions such as chronic bronchitis and emphysema cause the airways to constrict. As a result, there is a restriction in the airflow to and from the lungs, which results in dyspnea, or shortness of breath. When performing pulmonary function testing, COPD is identified by its typical low airflow.

Chronic bronchitis is caused by inflammation in the major airways and lung damage. The characteristic of chronic bronchitis in the lung's airways is an increase in the quantity (hyperplasia) and size (hypertrophy) of the goblet cells and mucous glands of the airway. As a result, there is more mucus in the airways than usual, which contributes to their narrowing and results in a cough that produces phlegm. The walls of the airways have been infiltrated by inflammatory cells under a microscope. Inflammation is followed by remodeling and scarring, which thicken the walls and also cause the airways to become more restricted. Squamous metaplasia (an abnormal change in the tissue lining the inside of the airway) and fibrosis (additional thickening and scarring of the airway wall) occur when chronic bronchitis worsens. These modifications have the effect of restricting airflow and making breathing difficult.

The airways are inflamed and constricted when someone has asthma, a chronic lung condition. Chronic wheezing, chest tightness, shortness of breath, and coughing are symptoms of asthma. Swelling and excessive mucus production can further narrow the airways and exacerbate symptoms. According to Roberts et al. (1995) Chest 107:111 S-117S, which is entirely included herein by reference, there is evidence that enhanced matrix degradation may occur in asthma and that this may contribute to mechanical alterations in the airways. Asthma symptoms may improve with extracellular matrix degradation treatment.

A multisystem recessive genetic disorder called cystic fibrosis causes improper chloride and sodium transport across epithelium, which results in thick, viscous secretions in the lungs, pancreas, liver, colon, and reproductive tract. A change in the gene encoding the cystic fibrosis transmembrane conductance regulator (CFTR) protein results in the disease. The clogging of the airways brought on by an accumulation of mucus, a reduction in mucociliary clearance, and the ensuing inflammation that arises from this can lead to lung fibrosis and anatomical abnormalities. Some cystic fibrosis patients need lung transplants because the fibrotic lung damage worsens over time.

Thick mucus buildup, copious phlegm production, frequent chest infections, coughing, shortness of breath, inflammation, decreased ability to exercise, opportunistic infections of the lung and sinus (including but not limited to Staphylococcus aureus, Haemophilus influenzae, Mycobacterium aviium, and Pseudomonas aeruginosa), pneumonia, tuberculosis, and bronchitis are just

Other uses for the compounds of Formula (I) or Formula (II) include the treatment or prevention of fibrotic diseases, disorders, or problems brought on by the development of post-operative adhesions. Surgery complications such post-surgical adhesion development are frequent. Adhesions that develop as a result of mechanical injury, ischemia, and infections may raise morbidity and mortality rates after surgery. Even though skilled surgical techniques can lessen the severity of adhesion formation, adhesions are seldom eliminated, necessitating the use of an efficient adjuvant therapy. Reducing the fibrosis brought on by this procedure may help with healing and recovery by lowering discomfort, blockage, and other surgical consequences.

Mammalian tissue injuries, such as lacerations and openings, cause tissue disruption and coagulation of the microvasculature at the wound face. Such tissue repair is an organized, well-controlled cellular reaction to harm. No of their size, soft tissue injuries heal similarly. Cellular proliferation and angiogenesis take place in the presence of an oxygen gradient in the biologic systems responsible for tissue growth and repair. According to Hunt, T. K., et al., “Coagulation and macrophage stimulation of angiogenesis and wound healing,” in The Surgical Wound, pp. 1-18, ed. F. Dineen & G. Hildrick-Smith (Lea & Febiger, Philadelphia: 1981), the sequential morphological and structural changes that take place during tissue repair have been characterized in detail and in some cases quantified. The cell morphology is divided into three different zones. The core avascular wound region exhibits high lactate levels, is acidotic, hypercarbic, and oxygen-deficient. A gradient zone of local anemia (ischemia) that is home to dividing fibroblasts is present next to the wound area. A region of active collagen production, or neovascularization, can be found behind the leading zone. The U.S. Pat. Nos. 5,015,629 and 7,022,675 (each incorporated herein by reference) describe procedures and materials for quickening wound healing.

By giving the compounds of Formula (I) or Formula (II) to a patient who needs therapy, in some embodiments, it is possible to lessen or stop the formation of scars in that patient. Scarring is a typical side effect of healing. Excessive, thick, or elevated scarring can develop in a wound as a result of disorderly collagen production and deposition. In general, larger wounds take longer to heal and are more likely to leave troublesome scars.

In other embodiments, the compounds of Formula (I) or Formula (II) can be used to lessen or stop the development of skin or scleroderma scars. Scars on skin can take a variety of forms. Raised, pinkish-red regions inside the boundaries of the initial damage are known as hypertropic scars. Hypertropic scars can sometimes naturally fade and diminish. Raised, intensely red spots known as keloids typically cover a larger region than the original damage. Keloids typically come back, even after surgical removal. Skin depressions known as atrophic scars can occasionally result from severe acne. They are brought on by inflammation, which breaks down the collagen while it is being rebuilt and leaves an area of indentation.

The compounds of Formula (I) or Formula (II) may be employed to treat or prevent systemic sclerosis in some embodiments. A systemic connective tissue illness called systemic sclerosis is characterized by changes in the microvasculature, immune system modifications, and a significant buildup of collagen and other matrix materials in the connective tissue. The connective tissue of the skin and internal organs like the gastrointestinal tract, lungs, heart, and kidneys are affected by systemic sclerosis, a clinically heterogeneous global condition. Reducing the fibrosis brought on by systemic sclerosis may lessen symptoms and/or stop additional problems in the tissues involved.

In other embodiments, the compounds of Formula (I) or Formula (II) can be used to treat or prevent liver fibrosis. Liver fibrosis can result from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha-1-antitrypsin deficiency, primary biliary cirrhosis, drug reaction and exposure to toxins.

NASH is a common liver disease. It resembles alcoholic liver disease but occurs in people who drink little or no alcohol. Fat in the liver, together with inflammation and damage, is the main aspect of NASH. NASH can, however, progress to cirrhosis, a condition in which the liver is permanently scarred and damaged and is no longer capable of functioning normally.

NASH is usually a silent disease with few or no symptoms. Patients typically experience no symptoms in the early stages of the disease and only start to experience symptoms, such as weakness, fatigue, and weight loss, if the disease has progressed or cirrhosis has formed. NASH can develop over several years or even decades. Without therapy, the process can halt and, in some cases, even start to reverse on its own. In some cases, NASH may gradually get worse, resulting in the development of liver fibrosis or scarring. As fibrosis progresses, cirrhosis emerges, causing the liver to become severely scarred, hardened, and incapable of performing its normal functions. Although cirrhosis does not always occur in NASH patients, once there is substantial scarring or cirrhosis, there are few medications that can stop its progression. Cirrhosis patients experience fluid retention, muscular atrophy, gastrointestinal bleeding, and liver failure. Advanced cirrhosis with liver failure can only be treated with a liver transplant, which is increasingly performed on NASH patients.

The compounds of Formula (I) or Formula (II) may be employed to cure or prevent renal fibrosis in some embodiments. Dialysis after kidney failure, catheterization, nephropathy, glomerulosclerosis, glomerulonephritis, chronic renal insufficiency, acute kidney injury, end-stage renal disease, or renal failure can all lead to kidney fibrosis.

Kidney (renal) fibrosis is caused by an excessive buildup of fibrous connective tissue. Dialysis or a kidney transplant may be necessary as a result of renal fibrosis, which has a major impact on morbidity and mortality. The filtering or reabsorptive portion of the nephron, the kidney's functional unit, can develop fibrosis. Scarring in the kidneys can be caused by a variety of things, most notably physiologic disorders that affect the glomerular filtration autoregulation. This then results in extracellular matrix building up and replacing normal structures. Numerous peptide and non-peptide fibrogens are produced as a result of a variety of physiologic changes in individual cells, which alter the ratio of extracellular matrix synthesis to breakdown in a way that favors scarring.

In some instances, inflammation may be one of the signs of organ fibrosis. According to these embodiments, a therapeutically effective dose of the compounds of Formula (I) or Formula (II) given to the subject in need of it can be one that decreases or reduces the number of inflammatory cells in the tissue or organ. To find out whether the number of inflammatory cells has decreased or increased, a pertinent sample can be taken from the subject. In a non-limiting embodiment, a decrease in neutrophil count in bronchoalveolar lavage (BAL) fluid from the cystic fibrosis (CF) subject may be used to demonstrate the positive effect. A major treatment target for CF patients is the excessive recruitment of neutrophils into their airways, which is a strong predictor of the severity of their lung illness.

In other embodiments, a decrease in one or more inflammatory biomarkers in a pertinent sample from the subject may be used to measure the positive effect of the compounds of Formula (I) or Formula (II). The inflammatory biomarker may include one or more cytokines or inflammatory cytokines linked to fibrosis in a variety of non-limiting embodiments. In BAL fluid, these cytokines may include, for instance, IL1, MIP2 (such as CCL3 or CCL4), IFN, TGF, TNF, IL-6, MCP-1, IL2, and IL-10. ELISAs are just one of many widely used techniques for determining the concentration of these biomarkers. Therefore, in one example, the procedures may further include lowering the level of one or more inflammatory biomarkers in a subject sample when compared to the control.

In other examples, a method for lowering or reducing collagen secretion or collagen deposition in a tissue or organ, such as the lung, the liver, the skin, or the heart of a subject, can make use of the compounds of Formula (I) or Formula (II). The procedure may involve giving the patient who requires it a dose of the compounds of Formula (I) or Formula (II) that is clinically effective. The person may have excessive collagen secretion or deposition in the tissue or organ, such as the kidney, lung, liver, intestines, colon, skin, or heart, or may be at risk for such conditions. Usually, an injury or an insult is the cause of an organ's excessive collagen secretion or deposition. These harms and insults target particular organs. The amount of collagen deposition in the tissue or organ can be totally or partially decreased or reduced by using the compounds of Formula (I) or Formula (II) over a long enough length of time. A week, a week to a month, a month to two months, or even more than two months can be considered a sufficient amount of time. The compounds of Formula (I) or Formula (II) can be used for the rest of a subject's life when treating chronic conditions.

### Atopic Dermatitis

In some aspects, provided are methods for treating eczema in a subject in need thereof, comprising administering to the subject a composition comprising a therapeutically effective amount of the compounds of Formula (I) or Formula (II), a pharmaceutically acceptable salt thereof or a pharmaceutical composition of the compounds of Formula (I) or Formula (II).

In some variations, the eczema is atopic dermatitis. Atopic dermatitis is a condition that makes the skin of the affected subject red and itchy. The atopic dermatitis may be chronic and can flare periodically. Atopic dermatitis symptoms may include, for example, dry skin; itching; red to brownish-gray patches; small, raised bumps, which may lead fluid and/or crust over when scratched; scaly skin; sensitive skin from scratching. A subject suffering from atopic dermatitis may also experience other conditions, such as asthma or hay fever. Atopic dermatitis may affect children, adolescents, and adults.

In some variations, the compositions herein are used to treat eczema, including for example, atopic dermatitis, in humans and other animals with naturally occurring eczema.

In some embodiments, the composition administered is a personal care product. In one variation, the composition administered is a cosmetic product, a drug product, or both. In certain variations, the composition administered meets US Food and Drug Administration regulations, including requirements defining a cosmetic product, a drug product, or both.

In some variations, the methods provided herein alleviate, or slow or stop the development or progression of one or more symptoms caused by or associated with eczema, including for example, atopic dermatitis. In other variations, the methods provided herein reduces the extent of eczema, or relieves eczema by causing the regression of one or more symptoms.

In some embodiments, a “therapeutically effective amount” refers to an amount effective, when administered to the subject, to treat a disease, e.g., a therapeutically effective amount may be an amount sufficient to treat eczema, including for example, atopic dermatitis. The therapeutically effective amount may be ascertained experimentally.

In some embodiments, the composition is administered to at least a portion of the body, where the subject may be suffering from eczema, or one or more symptoms of eczema. In some embodiments, the composition is administered to at least a portion of a hand, arm, leg, face and neck, trunk of the subject, where the subject may be suffering from eczema, or one or more symptoms of eczema. In another embodiment, the composition is administered to at least a portion of the full body, where the subject may be suffering from eczema, or one or more symptoms of eczema. In yet another embodiment, the composition is administered to at least a portion of the full body, where the subject may expect to suffer from eczema, or expect one or more symptoms of eczema, but not yet suffer from eczema or one or more symptoms of eczema.

In some variations, the composition is administered to a subject having mild to severe eczema, including for example, atopic dermatitis. In some variations, the composition is administered to a subject having mild to moderate eczema, including for example, atopic dermatitis. In certain variations, about 5% to about 20% of the total body surface area of the subject, excluding the face, scalp, axillae, and intertriginous areas, is affected by eczema, including for example, atopic dermatitis.

In some embodiments, the administering of the composition to the subject reduces redness, average lesion size, itching or pain caused by the eczema (including for example, atopic dermatitis), or improves general complexion in areas affected by the eczema, or any combination of the foregoing.

In one aspect, provided is a method of reducing redness in a subject suffering from eczema, comprising administering to the subject any of the compositions comprising the compounds of Formula (I) or Formula (II), or pharmaceutical composition as described herein. In one variation of the foregoing aspect, the method reduces redness in a subject suffering from atopic dermatitis. In some embodiments, the redness is assessed on a 0 to 10 point scale, wherein 0 corresponds to no redness, and 10 corresponds to the worst redness imaginable. In some embodiments, the method comprises reducing redness by about 10 points, 9 points, 8 points, 7 points, 6 points, 5 points, about 4 points, about 3 points, about 2 points, or about 1 point from a baseline redness in the subject assessed prior to administration of the composition. In some embodiments, the method comprises reducing redness by about 100%, about 90%, about 80%, about 70%, about 60%, about 50%, about 40%, about 30%, about 20%, or about 10% from a baseline redness in the subject assessed prior to administration of the composition. In some embodiments, the method comprises administering the composition for a period of time sufficient to reduce redness by about 10 points, 9 points, 8 points, 7 points, 6 points, 5 points, about 4 points, about 3 points, about 2 points, or about 1 point from a baseline redness in the subject assessed prior to administration of the composition. In some embodiments, the method comprises administering the composition for a period of time sufficient to reduce redness by about 100%, about 90%, about 80%, about 70%, about 60%, about 50%, about 40%, about 30%, about 20%, or about 10% from a baseline redness in the subject assessed prior to administration of the composition.

In one aspect, provided is a method of reducing the severity of eczema in a subject suffering from eczema, comprising administering to the subject any of the compositions comprising the compounds of Formula (I) or Formula (II), or pharmaceutical composition as described herein. In one variation of the foregoing aspect, the method reduces the severity of atopic dermatitis in a subject suffering from atopic dermatitis. In some embodiments, the severity is assessed on a 0 to 4 point scale, wherein 0 corresponds to clear, and 4 corresponds to severe. In some embodiments, the severity is assessed on the basis of one or more criteria selected from the group consisting of erythema, induration or papulation, and oozing or crusting. In some embodiments, the severity is assessed on a 0 to 4 point Investigator Global Assessment (IGA) scale, as detailed in Table 5 herein. In some embodiments, the severity is assessed on a 0 to 4 point Participant Global Assessment (PGA) scale. In some embodiments of the foregoing, the severity of eczema is assessed on a 0 to 72 point Eczema Area and Severity Index Score (EASI). In some embodiments, the method comprises reducing severity by about 4 points, about 3 points, about 2 points, or about 1 point on the IGA scale from a baseline severity in the subject assessed prior to administration of the composition. In some embodiments, the method comprises reducing severity by about 4 points, about 3 points, about 2 points, or about 1 point on the PGA scale from a baseline severity in the subject assessed prior to administration of the composition. In some embodiments, the method comprises reducing severity by about 72 points, about 64 points, about 56 points, about 48 points, about 40 points, about 32 points, about 24 points, about 26 points, or about 8 points on the EASI scale from a baseline severity in the subject assessed prior to administration of the composition. In some embodiments, the method comprises reducing severity by about 100%, about 90%, about 80%, about 70%, about 60%, about 50%, about 40%, about 30%, about 20%, or about 10% from a baseline severity in the subject assessed prior to administration of the composition. In some embodiments, the method comprises administering the composition for a period of time sufficient to reduce severity by about 4 points, about 3 points, about 2 points, or about 1 point on the IGA scale from a baseline severity in the subject assessed prior to administration of the composition. In some embodiments, the method comprises administering the composition for a period of time sufficient to reduce severity by about 4 points, about 3 points, about 2 points, or about 1 point on the PGA scale from a baseline severity in the subject assessed prior to administration of the composition. In some embodiments, the method comprises administering the composition for a period of time sufficient to reduce severity by about 72 points, about 64 points, about 56 points, about 48 points, about 40 points, about 32 points, about 24 points, about 26 points, or about 8 points on the EASI scale from a baseline severity in the subject assessed prior to administration of the composition. In some embodiments, the method comprises administering the composition for a period of time sufficient to reduce severity by about 100%, about 90%, about 80%, about 70%, about 60%, about 50%, about 40%, about 30%, about 20%, or about 10% from a baseline severity in the subject assessed prior to administration of the composition.

In one aspect, provided is a method of improving the appearance of the skin of a subject suffering from eczema, comprising administering to the subject any of the compositions comprising the compounds of Formula (I) or Formula (II), or pharmaceutical composition as described herein. In one variation of the foregoing aspect, the method improves the appearance the skin of a subject suffering from atopic dermatitis. In some embodiments, the improvement in the appearance of the skin is assessed by the subject suffering from atopic dermatitis. In some embodiments, the improvement of the appearance of the skin is measured on a 1 to 7 point Global Impressions of Change (GIC) scale. In some embodiments, the method results in skin that is very much improved, much improved, minimally improved, or not changed in appearance as compared to the appearance of the skin immediately prior to administration of the compositions. In some embodiments, the method comprises administering the composition for a period of time sufficient to result in skin that is very much improved, much improved, minimally improved, or not changed in appearance as compared to the appearance of the skin immediately prior to administration of the compositions.

In one aspect, provided is a method of decreasing the severity of a target lesion in a subject suffering from eczema, comprising administering to the subject any of the compositions comprising the compounds of Formula (I) or Formula (II), or pharmaceutical composition as described herein. In one variation of the foregoing aspect, the method decreases the severity of a target lesion in a subject suffering from atopic dermatitis. In some embodiments, the severity of the target lesion is assessed on the basis of one or more criteria selected from the group consisting of erythema, edema or papulation, oozing or crusting, excoriation, lichenification, and dryness. In some embodiments, the target lesion has an area of at least 10 cm².

In one aspect, provided is a method of decreasing average lesion size in a subject suffering from eczema, comprising administering to the subject any of the compositions comprising the compounds of Formula (I) or Formula (II), or pharmaceutical composition as described herein. In one variation of the foregoing aspect, the method decreases average lesion size in a subject suffering from atopic dermatitis. In some embodiments, the method comprises reducing average lesion size by at least about 100%, at least about 90%, at least about 80%, at least about 70%, at least about 60%, at least about 50%, at least about 40%, at least about 30%, at least about 20%, or at least about 10% from a baseline average lesion size in the subject as measured prior to administration of the composition. In some embodiments, the method comprises administering the composition for a period of time sufficient to reduce average lesion size by at least about 100%, at least about 90%, at least about 80%, at least about 70%, at least about 60%, at least about 50%, at least about 40%, at least about 30%, at least about 20%, or at least about 10% from a baseline average lesion size in the subject as measured prior to administration of the composition.

In one aspect, provided is a method of reducing itching in a subject suffering from eczema, comprising administering to the subject any of the compositions comprising the compounds of Formula (I) or Formula (II), or pharmaceutical composition as described herein. In one variation of the foregoing aspect, the method reduces itching in a subject suffering from atopic dermatitis. In some embodiments, the itching is assessed on a 0 to 10 point Likert scale, wherein 0 corresponds to no itching, and 10 corresponds to the worst imaginable itch. In some embodiments, the itching is assessed based on the subject's experience over the past 24 hours. In some embodiments, the itching is assessed at 7 days, 14 days, 21 days, or 28 days following the start of administration of the composition. In some embodiments, the method comprises reducing the assessed itching by about 10 points, about 9 points, about 8 points, about 7 points, about 6 points, about 5 points, about 4 points, about 3 points, about 2 points, or about 1 point from a baseline assessment of itching in the subject prior to administration of the composition. In some embodiments, the method comprises reducing itching by about 100%, about 90%, about 80%, about 70%, about 60%, about 50%, about 40%, about 30%, about 20%, or about 10% from a baseline assessment of itching in the subject prior to administration of the composition. In some embodiments, the method comprises administering the composition for a period of time sufficient to reduce the assessed itching by about 10 points, about 9 points, about 8 points, about 7 points, about 6 points, about 5 points, about 4 points, about 3 points, about 2 points, or about 1 point from a baseline assessment of itching in the subject prior to administration of the composition. In some embodiments, the method comprises administering the composition for a period of time sufficient to reduce itching by about 100%, about 90%, about 80%, about 70%, about 60%, about 50%, about 40%, about 30%, about 20%, or about 10% from a baseline assessment of itching in the subject prior to administration of the composition.

In one aspect, provided is a method of reducing pain in a subject suffering from eczema, comprising administering to the subject any of the compositions comprising the compounds of Formula (I) or Formula (II), or pharmaceutical composition as described herein. In one variation of the foregoing aspect, the method reduces pain in a subject suffering from atopic dermatitis. In some embodiments, the pain is assessed on a 0 to 10 point Likert scale, wherein 0 corresponds to no pain, and 10 corresponds to the worst imaginable pain. In some embodiments, the pain is assessed based on the subject's experience over the past 24 hours. In some embodiments, the pain is assessed at 7 days, 14 days, 21 days, or 28 days following the start of administration of the composition. In some embodiments, the method comprises reducing the assessed pain by about 10 points, about 9 points, about 8 points, about 7 points, about 6 points, about 5 points, about 4 points, about 3 points, about 2 points, or about 1 point from a baseline assessment of pain in the subject prior to administration of the composition. In some embodiments, the method comprises reducing pain by about 100%, about 90%, about 80%, about 70%, about 60%, about 50%, about 40%, about 30%, about 20%, or about 10% from a baseline assessment of pain in the subject prior to administration of the composition. In some embodiments, the method comprises administering the composition for a period of time sufficient to reduce the assessed pain by about 10 points, about 9 points, about 8 points, about 7 points, about 6 points, about 5 points, about 4 points, about 3 points, about 2 points, or about 1 point from a baseline assessment of pain in the subject prior to administration of the composition. In some embodiments, the method comprises administering the composition for a period of time sufficient to reduce pain by about 100%, about 90%, about 80%, about 70%, about 60%, about 50%, about 40%, about 30%, about 20%, or about 10% from a baseline assessment of pain in the subject prior to administration of the composition.

In some variations of the foregoing, the subject is formally diagnosed or clinically diagnosed with eczema, including for example, atopic dermatitis.

### Manufacture of Medicaments

A method of making a compound, such as the compound of Formula (Iaa) comprises: providing a solution of isoandrographolide and a solvent; combining 2,2,6,6-tetramethylpiperidinyloxy (TEMPO), tetrabutylammonium iodide (TBAI), N-Chlorosuccinimide (NCS), a salt, and the solution to form a first intermediate; combining hydroxylamine hydrochloride, pyridine, and the first intermediate to form a second intermediate; and dehydration into cyanide of the second intermediate to form the compound of Formula (Iaa).

In some embodiments, the step of dehydration into cyanide, comprises combining triethylamine, 4-toluenesulfonyl chloride, and the second intermediate to form the compound of Formula (Iaa). In other embodiments, the step of dehydration into cyanide, comprises combining benzotriazol-1-yloxytripyrrolidinophosphonium hexafluorophosphate (PyBOP), 1,8-Diazabicyclo[5.4.0]undec-7-ene (DBU), and the second intermediate to form the compound of Formula (Iaa).

In certain embodiment, a method of making a compound, such as the compound is of Formula (IIaa), comprises: providing a solution of isoandrographolide and a solvent; combining tert-Butyldimethylsilyl chloride (TBDMSCl), and the solution to form a first intermediate; combining pyridine, celite, pyridinium chlorochromate, and the first intermediate to form a second intermediate; combining hydroxylamine hydrochloride, pyridine, and the second intermediate to form a third intermediate; and combining 4-toluenesulfonyl chloride and the third intermediate followed by treatment with dioxane HCL to form the compound of Formula (IIaa).

In other embodiment, a method of making a compound, such as the compound is of Formula (IIaa), comprises: providing a solution of isoandrographolide and a solvent; combining Dess-Martin Periodinane (DMP), and the solution to form a first intermediate; combining hydroxylamine hydrochloride, pyridine, and the first intermediate to form a second intermediate; and combining triethylamine, 4-methylbenzene-1-sulfonyl chloride, 4-dimethylaminopyridine (DMAP), and the second intermediate, followed by treatment with dioxane HCl to form the compound of Formula (IIaa).

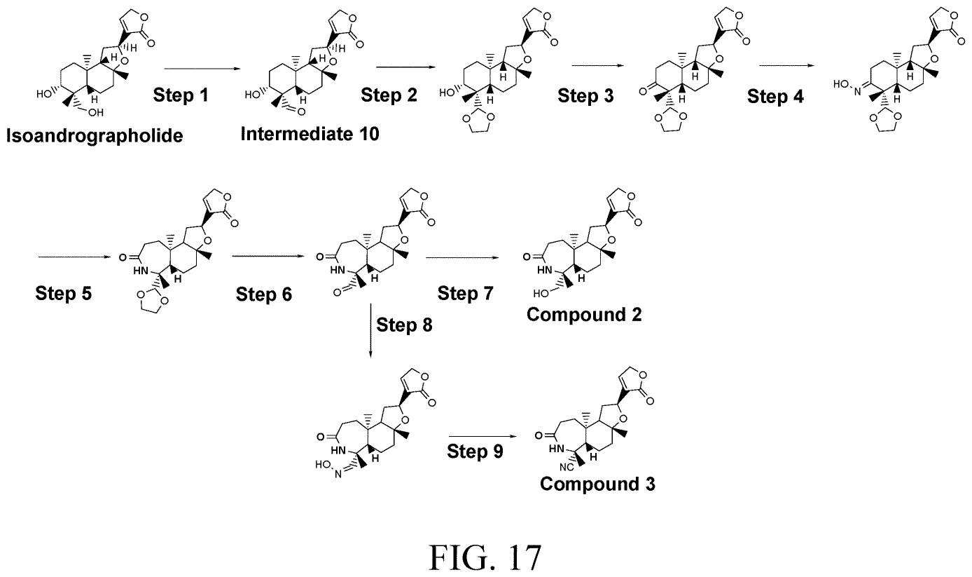

In other embodiments, a method of making a compound, such as Compound 3 as described below is shown in FIG. 17.

### Administration of Pharmaceutically Acceptable Salts and Compositions

In certain embodiments of the disclosure an “effective amount” of the compound, a pharmaceutically acceptable salt thereof or pharmaceutically acceptable composition is that amount effective for treating or lessening the severity of one or more of chronic pain, gut pain, neuropathic pain, musculoskeletal pain, acute pain, inflammatory pain, cancer pain, idiopathic pain, pruritis, atopic dermatitis, or psoriasis.

The compound of Formula (I) or Formula (II) and compositions, according to the method of the disclosure, may be administered using any amount and any route of administration effective for treating or lessening the severity of one or more of the pain or non-pain diseases recited herein. The exact amount required will vary from subject to subject, depending on the species, age, and general condition of the subject, the severity of the infection, the particular agent, its mode of administration, and the like. The compound of Formula (I) or Formula (II) of the disclosure may be formulated in dosage unit form for ease of administration and uniformity of dosage. The expression “dosage unit form” as used herein refers to a physically discrete unit of agent appropriate for the subject to be treated. It will be understood, however, that the total daily usage of the compound of Formula (I) or Formula (II) and compositions of the disclosure will be decided by the attending physician within the scope of sound medical judgment. The specific effective dose level for any particular subject or organism will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed, and like factors well known in the medical arts. The term “subject” or “patient,” as used herein, means an animal, preferably a mammal, and most preferably a human.

The pharmaceutically acceptable compositions of this disclosure can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. The phrases “parenteral administration” and “administered parenterally” as used herein means modes of administration other than enteral and topical administration, usually by injection, and includes, without limitation, intravenous, intramuscular, intraarterial, intrathecal, intracapsular, infraorbital, intracardiac, intradermal, intraperitoneal, transtracheal, subcutaneous, subcuticular, intraarticular, subcapsular, subarachnoid, intraspinal and intrasternal injection and infusion. In certain embodiments, the compound of Formula (I) or Formula (II) of the disclosure may be administered orally or parenterally at dosage levels of about 0.01 mg/kg to about 100 mg/kg, about 0.01 mg/kg to about 50 mg/kg and optionally from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

It is known that sterols, such as cholesterol, will form complexes with cyclodextrins. Thus, in certain embodiments, where the inhibitor is a steroidal alkaloid, it may be formulated with cyclodextrins, such as α-, β- and γ-cyclodextrin, dimethyl-β cyclodextrin and 2-hydroxypropyl-β-cyclodextrin.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, by autoclave sterilization, or by incorporating sterilizing agents in the form of sterile solid compositions which can be dissolved or dispersed in sterile water or other sterile injectable medium prior to use. In various embodiments, a first portion of the formulation may be sterilized by filtration (e.g., the compound) and the second portion may be sterilized by autoclave sterilization (e.g., the excipient).

In order to prolong the effect of a compound of the disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Formulations of the pharmaceutical compositions of the disclosure for rectal, vaginal, or urethral administration may be presented as a suppository, which may be prepared by mixing one or more compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and with any preservatives, buffers, or propellants that may be required.

Dosage forms for the topical or transdermal administration of a compound include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulation, eye ointments, eye drops, and eardrops are also contemplated as being within the scope of this disclosure. Pharmaceutical compositions suitable for parenteral administration comprise one or more compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

When the compound of Formula (I) or Formula (II) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compound of Formula (I) or Formula (II) employed in the pharmaceutical composition at levels lower than that required in order to achieve the desired therapeutic effect and gradually increase the dosage until the desired effect is achieved.

In general, a suitable daily dose of a compound will be that amount of the compound that is the lowest dose effective to produce a therapeutic effect. Such an effective dose will generally depend upon the factors described above. Generally, intravenous, intracerebroventricular and subcutaneous doses of the compound of Formula (I) or Formula (II) for a patient will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

The patient receiving this treatment is any animal in need, including primates, in particular humans, and other mammals such as equines, cattle, swine and sheep; and poultry and pets in general.

The compound can be administered as such or in admixtures with pharmaceutically acceptable and/or sterile carriers and can also be administered in conjunction with other antimicrobial agents such as penicillins, cephalosporins, aminoglycosides and glycopeptides. Conjunctive therapy thus includes sequential, simultaneous and separate administration of the active compound in a way that the therapeutic effects of the first administered one are still detectable when the subsequent therapy is administered.

The present invention contemplates formulation of the subject compounds in any of the aforementioned pharmaceutical compositions and preparations. Furthermore, the present invention contemplates administration via any of the foregoing routes of administration. One of skill in the art can select the appropriate formulation and route of administration based on the condition being treated and the overall health, age, and size of the patient being treated.

The activity of a compound utilized in this disclosure as an inhibitor of the therapeutic targets may be assayed according to methods described generally in the Examples herein, or according to methods available to one of ordinary skill in the art.

### Additional Therapeutic Agent

It will also be appreciated that the compound of Formula (I) or Formula (II) and pharmaceutically acceptable compositions of the disclosure can be employed in combination therapies, that is, the compound of Formula (I) or Formula (II) and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: ursodeoxycholic acid (UDCA), rifampicin, cholestyramine, naltrexone, sertraline, phenobarbital, gabapentin, pregabalin, and ondansetron. Additionally, nondrug analgesic approaches may be utilized in conjunction with administration of one or more compounds of the disclosure. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp &Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following: (1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine; (2) a nonsteroidal antiinflammatory drug (NSAID), e.g. aspirin, diclofenac, diflunisal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac; (3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental; (4) a benzodiazepine having a sedative action, e.g. chlordiazepoxide, clorazepate, diazepam, flurazepam, lorazepam, oxazepam, temazepam or triazolam; (5) a histamine (H1) antagonist having a sedative action, e.g. diphenhydramine, pyrilamine, promethazine, chlorpheniramine or chlorcyclizine; (6) a sedative such as glutethimide, meprobamate, methaqualone or dichloralphenazone; (7) a skeletal muscle relaxant, e.g. baclofen, carisoprodol, chlorzoxazone, cyclobenzaprine, methocarbamol or orphenadrine; (8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone; (9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline; (10) a tricyclic antidepressant, e.g. desipramine, imipramine, amitriptyline or nortriptyline; (11) an anticonvulsant, e.g. carbamazepine (Tegretol®), lamotrigine, topiramate, lacosamide (Vimpat®) or valproate; (12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S); (13) a muscarinic antagonist, e.g oxybutynin, tolterodine, propiverine, tropsium chloride, darifenacin, solifenacin, temiverine and ipratropium; (14) a COX-2 selective inhibitor, e.g. celecoxib, rofecoxib, parecoxib, valdecoxib, deracoxib, etoricoxib, or lumiracoxib; (15) a coal-tar analgesic, in particular paracetamol; (16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan; (17) a vanilloid receptor agonist (e.g. resinferatoxin or civamide) or antagonist (e.g. capsazepine, GRC-15300); (18) a beta-adrenergic such as propranolol; (19) a local anaesthetic such as mexiletine; (20) a corticosteroid such as dexamethasone; (21) a 5-HT receptor agonist or antagonist, particularly a 5-HT1B/1D agonist such as eletriptan, sumatriptan, naratriptan, zolmitriptan or rizatriptan; (22) a 5-HT2A receptor antagonist such as R (+)-alpha-(2,3-dimethoxy-phenyl)-1-[2-(4-fluorophenylethyl)]-4-piperidinemethanol (MDL-100907); (23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine; (24) Tramadol®, Tramadol ER (Ultram ER®), Tapentadol ER (Nucynta®); (25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl) pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide; (26) an alpha-2-delta ligand such as gabapentin (Neurontin®), gabapentin GR (Gralise®), gabapentin, enacarbil (Horizant®), pregabalin (Lyrica®), 3-methyl gabapentin, (1 [alpha],3 [alpha],5 [alpha]) (3-amino-methyl-bicyclo[3.2.0]hept-3-yl)-acetic acid, (3S,5R)-3-aminomethyl-5-methyl-heptanoic acid, (3 S,5R)-3-amino-5-methyl-heptanoic acid, (3 S,5R)-3-amino-5-methyl-octanoic acid, (2S,4S)-4-(3-chlorophenoxy) proline, (2S,4S)-4-(3-fluorobenzyl)-proline, [(1R,5R,6S)-6-(aminomethyl) bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3 S,5R)-3-amino-5-methyl-nonanoic acid, (3 S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid; (27) a cannabinoid such as KHK-6188; (28) metabotropic glutamate subtype 1 receptor (mGluR1) antagonist; (29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone; (30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine; (31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine; (32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile; 2-[[(1R,3 S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3 S)-3-amino-4-hydroxy-1-(5-thiazolyl)butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide; (33) an acetylcholinesterase inhibitor such as donepezil; (34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid; (35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870; (36) a 5-lipoxygenase inhibitor, such as zileuton, 6-[(3-fluoro-5-[4-methoxy-3,4,5,6-tetrahydro-2H-pyran-4-yl]) phenoxy-methyl]-1-methyl-2-quinolone (ZD-2138), or 2,3,5-trimethyl-6-(3-pyridylmethyl)-1,4-benzoquinone (CV-6504); (37) a sodium channel blocker, such as lidocaine, lidocaine plus tetracaine cream (ZRS-201) or eslicarbazepine acetate; (38) an NaV1.7 blocker, such as XEN-402 and such as those disclosed in WO2011/140425; WO2012/106499; WO2012/112743; WO2012/125613 or PCT/US2013/21535 the entire contents of each application hereby incorporated by reference; (39) an NaV1.8 blocker, such as those disclosed in WO2008/135826 and WO2006/011050 the entire contents of each application hereby incorporated by reference; (40) a combined NaV1.7 and NaV1.8 blocker, such as DSP-2230 or BL-1021; (41) a 5-HT3 antagonist, such as ondansetron; (42) a TPRV 1 receptor agonist, such as capsaicin (NeurogesX®, Qutenza®); and the pharmaceutically acceptable salts and solvates thereof; (43) a nicotinic receptor antagonist, such as varenicline; (44) an N-type calcium channel antagonist, such as Z-160; (45) a nerve growth factor antagonist, such as tanezumab; (46) an endopeptidase stimulant, such as senrebotase; and (47) an angiotensin II antagonist, such as EMA-401.

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 10% to 100% of the amount normally present in a composition comprising that agent as the only therapeutically active agent.

The compound of Formula (I) or Formula (II) of this disclosure or pharmaceutically acceptable compositions thereof may also be incorporated into compositions for coating an implantable medical device, such as prostheses, artificial valves, vascular grafts, stents and catheters. Accordingly, the disclosure, in another aspect, includes a composition for coating an implantable device comprising a compound of the disclosure as described generally above, and in classes and subclasses herein, and a carrier suitable for coating said implantable device. In still another aspect, the disclosure includes an implantable device coated with a composition comprising a compound of the disclosure as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

Another aspect of the disclosure relates to inhibiting a therapeutic target activity in a biological sample or a subject, which method comprises administering to the subject, or contacting the biological sample with a compound of Formula (I) or Formula (II) or a composition comprising the compound. The term “biological sample,” as used herein, includes, without limitation, cell cultures or extracts thereof, biopsied material obtained from a mammal or extracts thereof, and blood, saliva, urine, feces, semen, tears, or other body fluids or extracts thereof.

Inhibition of a therapeutic target activity in a biological sample is useful for a variety of purposes that are known to one of skill in the art. Examples of such purposes include, but are not limited to, the study of therapeutic targets in biological and pathological phenomena; and the comparative evaluation of new therapeutic target inhibitors.

In some embodiments, the present disclosure relates to a kit that includes the composition and specific instructions that explain how to use the composition for treating, ameliorating, or preventing one or more conditions and diseases (e.g., pruritis) in a subject in need thereof.

Reference to “about” a value or parameter herein includes (and describes) embodiments that are directed to that value or parameter per se. For example, description referring to “about X” includes description of “X”. In some embodiments, the term “about” when used in association with a measurement, or used to modify a value, a unit, a constant, or a range of values, refers to variations of +/−10%, 5%, 2%, or 1%.

Reference to “between” two values or parameters herein includes (and describes) embodiments that include those two values or parameters per se. For example, description referring to “between x and y” includes description of “x” and “y” per se.

### EXAMPLES

The presently disclosed subject matter will be better understood by reference to the following examples, which are provided as exemplary of the invention, and not by way of limitation.

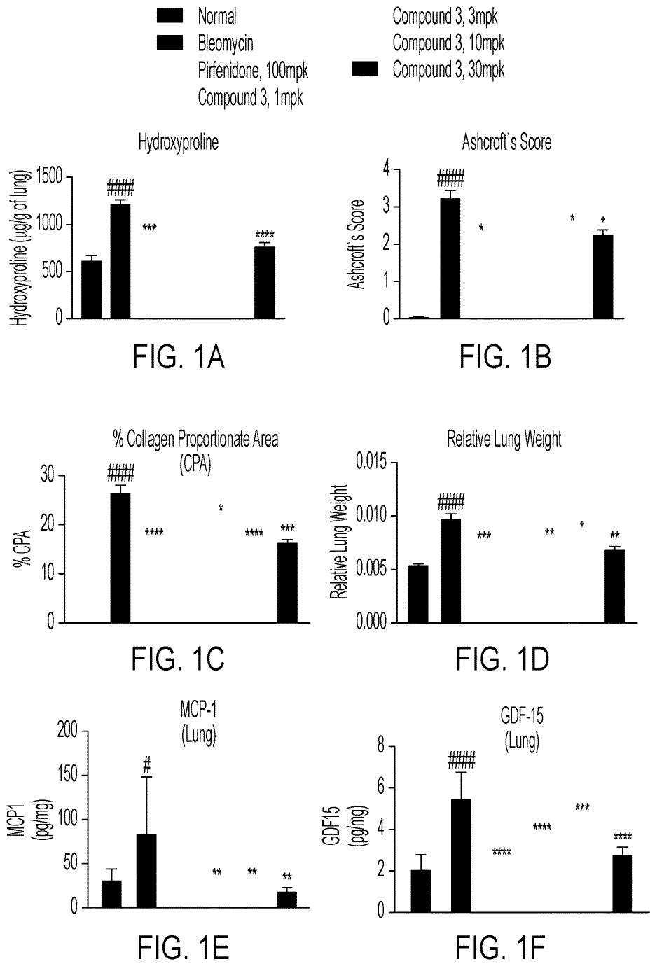

As referenced in FIGS. 1-17, Exemplary Compound I (also referred to herein as Compound 1) is:

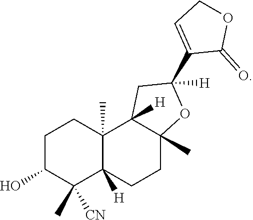

As referenced in FIGS. 1-17, Exemplary Compound II (also referred to herein as Compound 2) is:

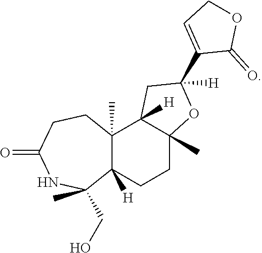

As referenced in FIGS. 1-17, Exemplary Compound III (also referred to herein as Compound 3) is:

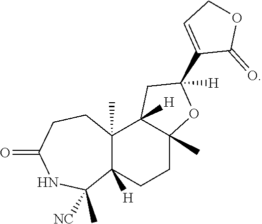

### Example 1

Detailed synthesis of exemplary compounds and intermediates as disclosed above is presented as detailed below:

TABLE 1
List of Compounds of Formula (I) excluding Isoandrographolide and
Andrographolide

| Compound No: | Structure |
|---|---|
| Isoandrographolide | 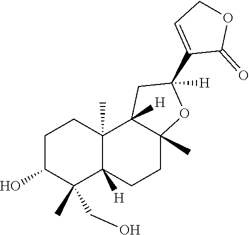 |
| Intermediate 1 | 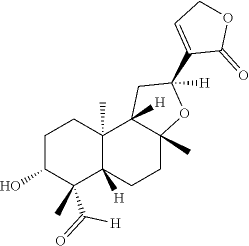 |
| Intermediate 2 | 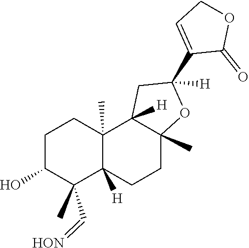 |
| Compound 1 | 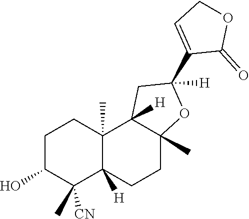 |
| Compound 2 | 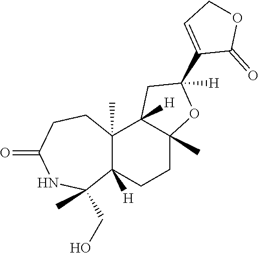 |
| Compound 3 | 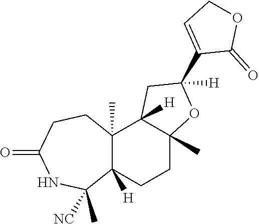 |
| Intermediate 3 | 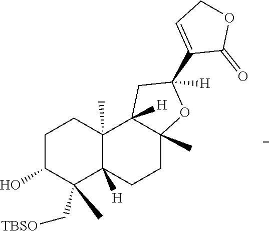 |
| Intermediate 4 | 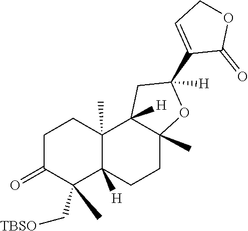 |
| Intermediate 5 | 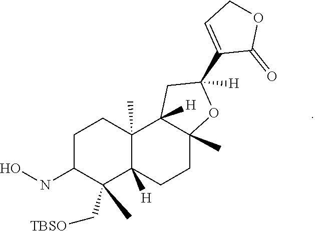 |
| Intermediate 6 | 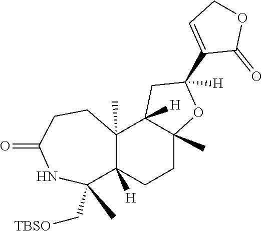 |
| Intermediate 7 | 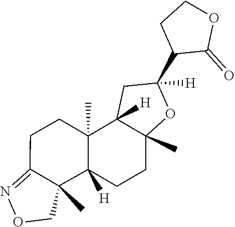 |
| Intermediate 8 | 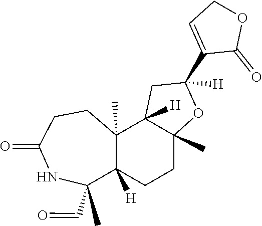 |
| Intermediate 9 | 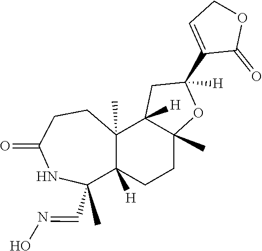 |
| Compound 4 | 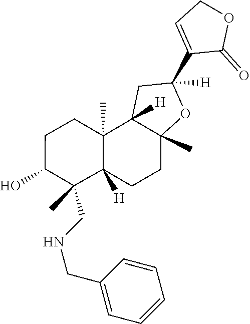 |
| Compound 5 | 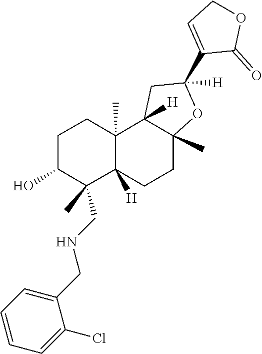 |
| Compound 6 | 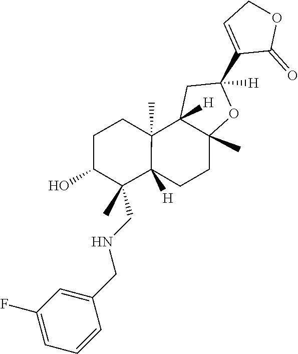 |
| Compound 7 | 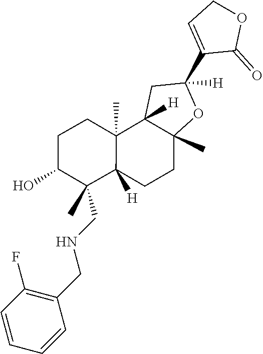 |
| Compound 8 | 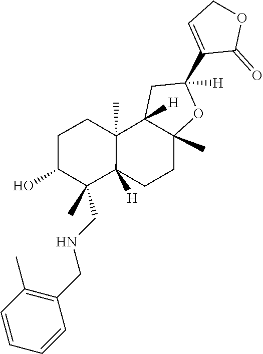 |
| Compound 9 | 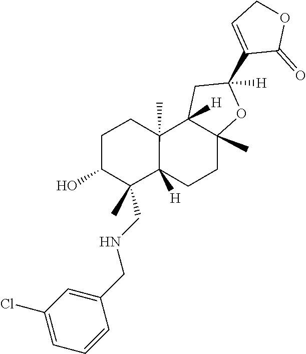 |
| Compound 10 | 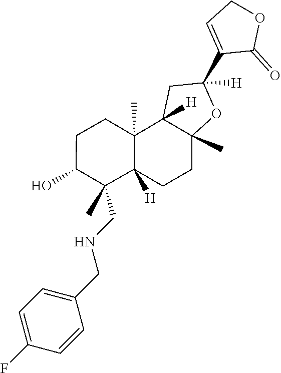 |
| Compound 11 | 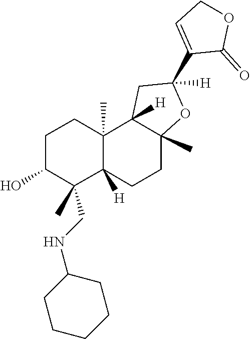 |
| Compound 12 | 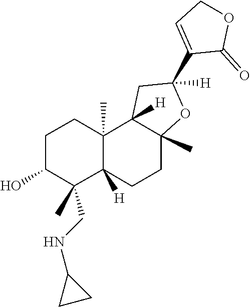 |
| Compound 13 | 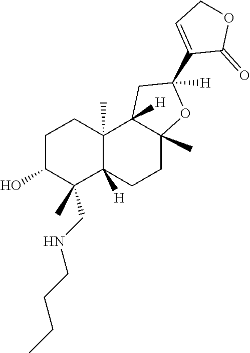 |
| Compound 14 | 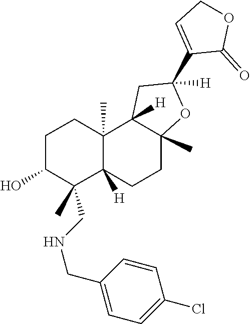 |
| Compound 15 | 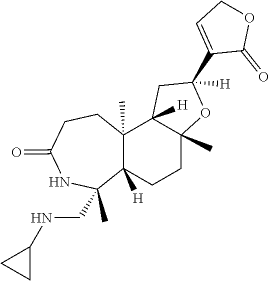 |
| Intermediate 10 | 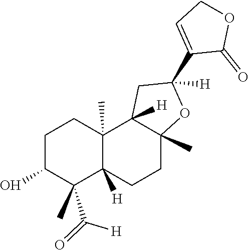 |
| Intermediate 11 | 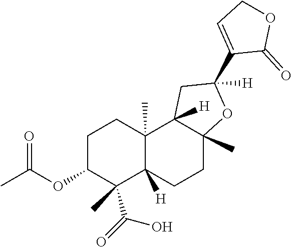 |
| Compound 16 | 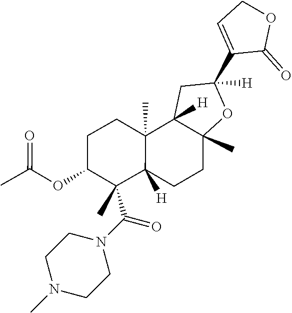 |
| Compound 17 | 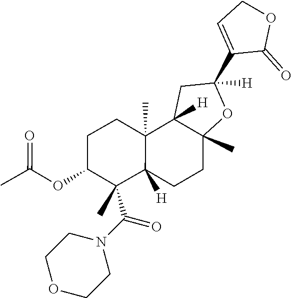 |
| Compound 18 | 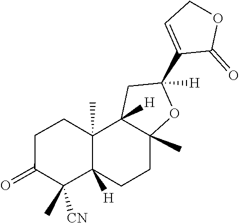 |
| Compound 19 | 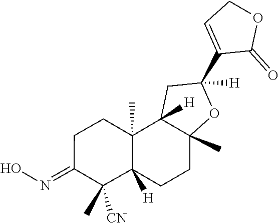 |
| Compound 20 | 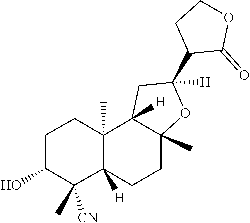 |
| Compound 21 | 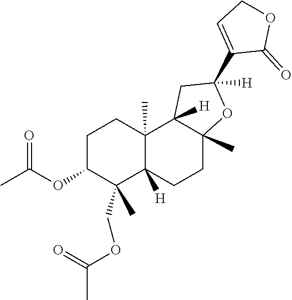 |
| Intermediate 12 | 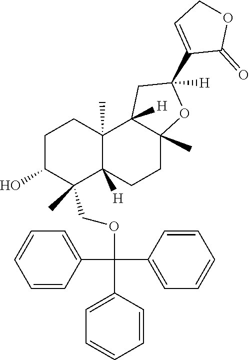 |
| Intermediate 13 | 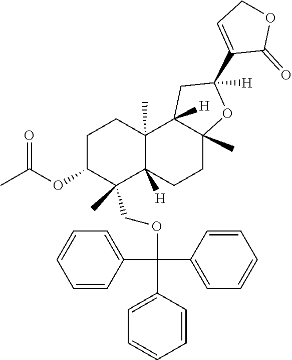 |
| Compound 22 | 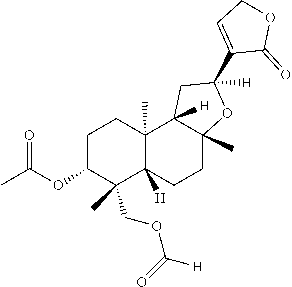 |
| Compound 23 | 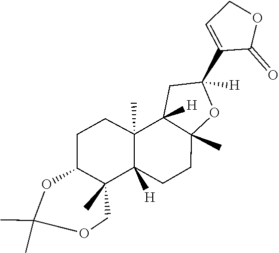 |
| Intermediate 14 | 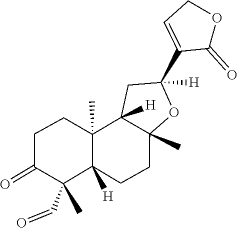 |
| Compound 24 | 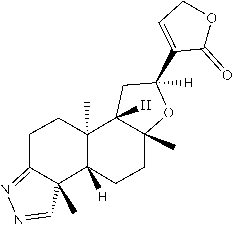 |
| Andrographolide | 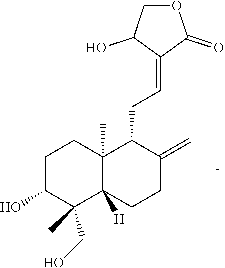 [SMILES: C=C1CC[C@@H]2[C@](C)(CO)[C@H](O)CC[C@@]2(C)[C@@H]1C/C=C1/C(=O)OCC1O] |
| Intermediate 15 | 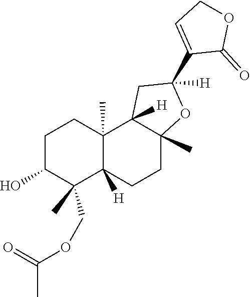 |
| Compound 25 | 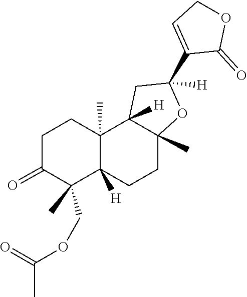 |
| Compound 26 | 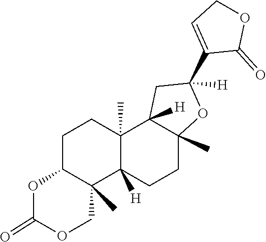 |
| Compound 27 | 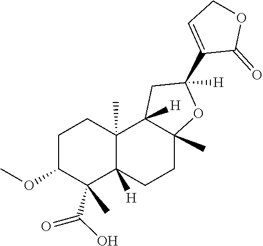 |
| Compound 28 | 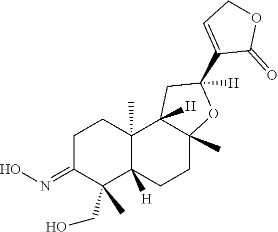 |
| Compound 29 | 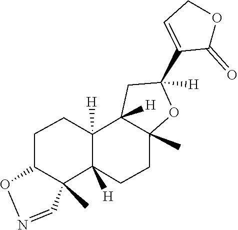 |
| Compound 30 | 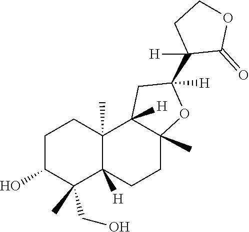 |
| Compound 31 | 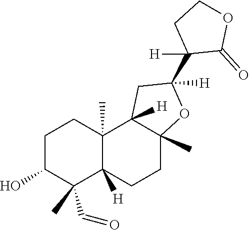 |

### Representative Synthesis of Compound 1 (Also Referred to Herein as Compound I)

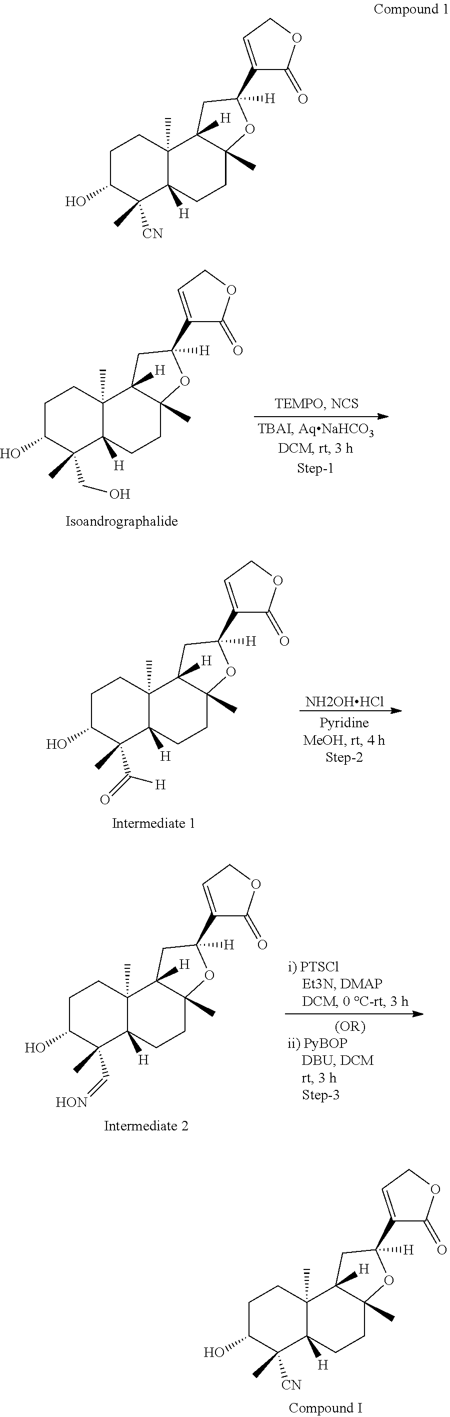

### Step 1: Synthesis of Intermediate 1

To a solution of 3-((2S,3aR,5aS,6R,7R,9aR,9bS)-7-hydroxy-6-(hydroxymethyl)-3a,6,9a-trimethyldodecahydronaphtho[2,1-b]furan-2-yl) furan-2(5H)-one (Isoandrographolide) (10.0 g, 28.5 mmol, 1.0 eq.) in DCM (100 mL) were added 2,2,6,6-Tetramethylpiperidinyloxy (TEMPO, 0.892 g, 5.71 mmol, 0.2 eq.), tetrabutylammonium iodide (TBAI, 2.11 g, 5.71 mmol, 0.2 eq.), N-chlorosuccinimide (NCS, 5.72 g, 42.8 mmol, 1.5 eq.), Aq. NaHCO₃ (0.5 M, 200 mL) and Aq. K₂CO₃ (0.05 M, 200 mL) cooled under ice bath. The resulting mixture was stirred at rt for 3 h. Progress of the reaction was monitored by TLC. After completion of the reaction, the organic layer was separated, and the aqueous layer was extracted with DCM thrice (3×100 mL). The combined organic layer was dried over Na₂SO₄, filtered, and concentrated under reduced pressure. The crude product was treated with hexane (100 mL) under stirring at rt for 15 min. Decanted the hexane layer and the residue obtained was treated methanol (30 mL) at 0 °C and stirred for 15 min., filtered, and washed with cold methanol (10 mL) and dried under vacuum to afford (2S,3aR,5aS,6S,7R,9aR,9bS)-7-hydroxy-3a,6,9a-trimethyl-2-(2-oxo-2,5-dihydrofuran-3-yl) dodecahydronaphtho[2,1-b]furan-6-carbaldehyde (Intermediate 1), as off white solid. Yield: 6.1 g (61.4%).

¹H NMR (400 MHz, CDCl₃, δ): 9.76 (d, J=2.0 Hz, 1H), 7.27 (q, J=2.0 Hz, 1H), 4.82-4.80 (m, 2H), 4.75-4.65 (m, 1H), 3.25-3.15 (m, 2H), 2.49-2.44 (m, 1H), 2.30-2.25 (m, 1H), 2.1-2.05 (m, 1H), 1.90-1.70 (m, 5H), 1.57-1.50 (m, 2H), 1.32 (s, 3H), 1.14-1.13 (m, 1H), 1.1 (s, 3H), 1.07-1.06 (m, 1H), 0.91 (s, 3H). 13C NMR (100 MHz, CDCl₃, δ): 207.9, 172.5, 143.2, 138.3, 82.3, 73.2, 70.5, 59.2, 56.4, 53.6, 39.1, 36.3, 36.0, 32.9, 31.2, 27.8, 24.3, 19.8, 18.1, 13.7.

### Step 1: Synthesis of Intermediate 1

To a solution of Isoandrographolide (500.0 g, 1.42 mol, 1.0 eq.) in DCM (5 L) were added TEMPO (44.5 g, 0.28 mol, 0.2 eq.), TBAI (105.0 g, 0.28 mol, 0.2 eq.), NCS (285.0 g, 2.14 mol, 1.5 eq.), aq. NaHCO₃ (0.5 M, 10 L, 20 vol.) and aq. K₂CO₃ (0.05 M, 10 L, 20 vol.) cooled under ice bath. The resulting reaction mixture was stirred at rt for 3 h. Progress of the reaction was monitored by TLC. After completion of the reaction, the organic layer was separated, and the aqueous layer was extracted with dichloromethane twice (2×5 L). The combined organic layer washed with brine solution (5 L) and concentrated under reduced pressure. Acetonitrile (1 L) was added to the crude and then stirred for 1 h at rt. The precipitated solid was filtered and washed with acetonitrile (0.5 L) followed by n-heptane (1 L), dried under vacuum to afford (2S,3aR,5aS,6S,7R,9aR,9bS)-7-hydroxy-3a,6,9a-trimethyl-2-(2-oxo-2,5-dihydrofuran-3-yl) dodecahydronaphtho[2,1-b]furan-6-carbal (Intermediate 1), as Off-white solid. Yield: 420.0 g (84.5%)

### Step 2: Synthesis of Intermediate 2

Hydroxylamine hydrochloride (8.38 g, 121 mmol, 3.5 eq.) and pyridine (27.7 mL, 344 mmol, 10.0 eq.) in methanol (50 mL) was added dropwise to a solution of (2S,3aR,5aS,6S,7R,9aR,9bS)-7-hydroxy-3a,6,9a-trimethyl-2-(2-oxo-2,5-dihydrofuran-3-yl) dodecahydronaphtho[2,1-b]furan-6-carbaldehyde (Intermediate 1) (12.0 g, 34.4 mmol, 1.0 eq.) in methanol (100 mL) at rt. The resulting mixture was stirred at rt for 4 h. Progress of the reaction was monitored by TLC. After completion of the reaction, concentrated the reaction mixture under reduced pressure and ice-cold water (150 mL) was added under stirring. The precipitated product was filtered and dried under vacuum to afford (E)-7-hydroxy-3a,6,9a-trimethyl-2-(2-oxo-2,5-dihydrofuran-3-yl) dodecahydronaphtho[2,1-b]furan-6-carbaldehyde oxime (Intermediate 2), as off-white solid. (9.0 g). Crude product was taken directly for the next step without further purification.

¹H NMR (400 MHz, CDCl₃, δ): 7.61 (brs, 1H), 7.44 (brs, 1H), 7.30-7.28 (m, 1H), 4.9-4.8 (m, 2H), 4.7-4.6 (m, 1H), 3.9-3.75 (m, 1H), 3.75-3.20 (m, 1H), 2.50 (t, J=9.2 Hz, 1H), 2.26 (d, J=10.4 Hz, 1H), 2.15-2.0 (m, 1H), 1.98-1.75 (m, 4H), 1.73-1.5 (m, 7H), 1.33 (s, 3H), 1.14 (s, 3H), 1.10-1.05 (m, 2H), 0.97 (s, 3H). 13C NMR (100 MHz, CDCl₃, δ): 172.5, 156.1, 143.1, 138.5, 82.5, 78.8, 73.3, 70.5, 56.9, 53.0, 44.4, 39.6, 36.0, 35.9, 32.8, 31.3, 27.6, 24.2, 18.1, 14.8.

### Step 2: Synthesis of Intermediate 2

(2S,3aR,5aS,6S,7R,9aR,9bS)-7-hydroxy-3a,6,9a-trimethyl-2-(2-oxo-2,5-dihydrofuran-3-yl) dodecahydronaphtho[2,1-b]furan-6-carbaldehyde (Intermediate 1) (420.0 g, 1.2 mol., 1.0 eq.) was added in lot wise to a solution of hydroxylamine hydrochloride (294.0 g, 4.21 mol., 3.5 eq.), pyridine (0.97 L, 12.0 mol., 10.0 eq.) in methanol (5 L, 12 vol.) at rt. The resulting reaction mixture was stirred at rt for 4 h. Progress of the reaction was monitored by TLC & LC-MS. After completion of the reaction, ice cold water (4.2 L) was added. The precipitated product was filtered and washed with heptanes (1.26 L). Compound swapped with toluene under vacuum below 48 °C, to afford (E)-7-hydroxy-3a,6,9a-trimethyl-2-(2-oxo-2,5-dihydrofuran-3-yl) dodecahydronaphtho[2,1-b]furan-6-carbaldehyde oxime (Intermediate 2), as off white solid. Yield: 325.0 g. Crude product was taken directly for the next step without further purification.

### Step 3: Synthesis of Compound 1

To a solution of (E)-7-hydroxy-3a,6,9a-trimethyl-2-(2-oxo-2,5-dihydrofuran-3-yl) dodecahydronaphtho[2,1-b]furan-6-carbaldehyde oxime (Intermediate 2) (1.7 g, 4.68 mmol, 1.0 eq.) in DCM (15 mL) was added triethylamine (1.96 mL, 14 mmol, 3.0 eq.) at 0 °C, were added 4-toluenesulfonyl chloride (1.34 g, 7.02 mmol, 1.5 eq.) and catalytic amount of 4-dimethylaminopyridine (57.1 mg, 0.468 mmol, 0.1 eq.). The resulting reaction mixture was allowed to warm to rt and stirred at rt for 3 h. Progress of the reaction was monitored by TLC. After completion of the reaction, diluted the reaction mixture with DCM (100 mL) and washed with water thrice (3×30 mL). The combined organic layer was dried over Na₂SO₄, filtered, and concentrated under reduced pressure. The crude product obtained was purified by column chromatography over silica gel (100-200 mesh) using mixtures of ethyl acetate and hexanes as eluent to afford (2S,3aR,5aS,6R,7R,9aR,9bS)-7-hydroxy-3a,6,9a-trimethyl-2-(2-oxo-2,5-dihydrofuran-3-yl) dodecahydronaphtho[2,1-b]furan-6-carbonitrile (Compound 1), as white solid. Yield: 0.6 g (37.1%).

¹H NMR (400 MHz, CDCl₃, δ): 7.32 (q, J=1.6 Hz, 1H), 4.82 (q, J=1.6 Hz, 2H), 4.8-4.7 (m, 1H), 3.3-3.2 (m, 1H), 2.50 (q, J=8.0 Hz, 1H), 2.35-2.25 (m, 1H), 2.1-1.94 (m, 2H), 1.92-1.70 (m, 4H), 1.68-1.56 (m, 5H), 1.53 (s, 3H), 1.27 (s, 3H), 1.09-0.98 (m, 2H). 13C NMR (100 MHz, CDCl₃, δ): 172.9, 146.4, 136.2, 123.2, 82.4, 74.8, 72.5, 71.1, 56.8, 48.5, 42.8, 38.0, 35.8, 34.6, 32.3, 31.4, 28.2, 23.8, 19.4, 14.4. LC-MS: m/z 346.2 (M+H).

### Step 3: Synthesis of Compound 1

To a solution of (E)-7-hydroxy-3a,6,9a-trimethyl-2-(2-oxo-2,5-dihydrofuran-3-yl) dodecahydronaphtho[2,1-b]furan-6-carbaldehyde oxime (Intermediate 2) (350.0 g, 0.96 mol., 1.0 eq.) in Dichloromethane (4.2 L, 12 vol.) were added PyBOP (1.0 Kg, 1.92 mol., 2.0 eq.) at rt followed by (5 min gap) DBU (337.0 g, 2.21 mol., 2.3 eq.) (Observation: Reaction mixture became a clear homogeneous solution). The resulting mixture was stirred at rt for 3 h. Progress of the reaction was monitored by TLC. After completion of the reaction, the reaction mixture is poured into water (10 vol.) and extracted with dichloromethane twice (2×500 mL). The combined organic layer was washed with saturated brine solution (3.5 L) and concentrated under reduced pressure. The product obtained was treated with ethyl acetate (3.5 L, 10 vol.) and stirred at rt for 2 h. Filtered the product and washed with ethyl acetate (700 mL) and dried under vacuum to afford (2S,3aR,5aS,6R,7R,9aR,9bS)-7-hydroxy-3a,6,9a-trimethyl-2-(2-oxo-2,5-dihydrofuran-3-yl) dodecahydronaphtho[2,1-b]furan-6-carbonitrile (Compound 1), as white solid. Yield: 215.0 g (64.7%).

### Representative Synthesis of Compound 2 (Also Referred to Herein as Compound II)

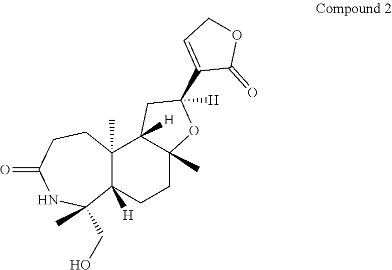

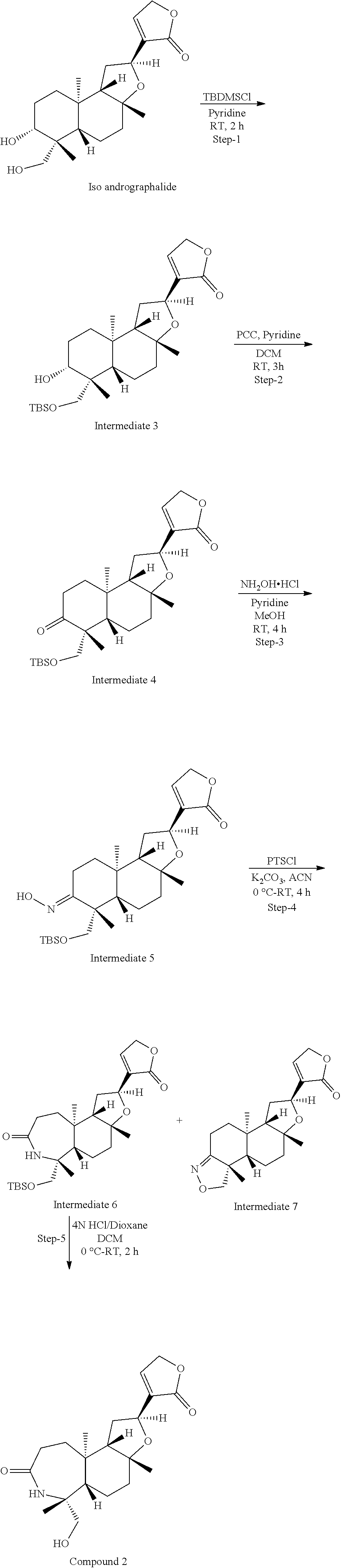

### Step 1: Synthesis of Intermediate 3

To a solution of 3-((2S,3aR,5aS,6R,7R,9aR,9bS)-7-hydroxy-6-(hydroxymethyl)-3a,6,9a-trimethyldodecahydronaphtho[2,1-b]furan-2-yl) furan-2(5H)-one (Isoandrographolide) (2.0 kg, 5.7 mol., 1.0 eq.) in pyridine (4.8 L, 2.4 vol.) was added TBDMS-Cl (3.44 kg, 22.8 mol., 4.0 eq.) at rt. The resulting mixture was stirred at rt for 3 h. Progress of the reaction was monitored by TLC & LCMS. After completion of the reaction, cold water (40 L) and heptane (20 L) were added to the reaction mixture and stirred at rt for 1 h, filtered, washed with heptane (4 L). Compound dried under vacuum to afford 3-((2S,3aR,5aS,6R,7R,9aR,9bS)-6-(((tert-butyl dimethylsilyl)oxy)methyl)-7-hydroxy-3a,6,9a-trimethyldodecahydronaphtho[2,1-b]furan-2-yl) furan-2(5H)-one (Intermediate 3) (1.1 kg), as an off-white solid. Organic layers were combined and washed with cold water (20 L, 10 vol.), brine solution (20 L, 10 vol.) and concentrated under reduced pressure. The crude product obtained was treated with Heptane (4 L) under stirring at 0 °C for 1 h. Filtered the solids and washed with cold Heptane (0.5 L), dried under vacuum to afford 3-((2S,3aR,5aS,6R,7R,9aR,9bS)-6-(((tert-butyldimethylsilyl)oxy)methyl)-7-hydroxy-3a,6,9a-trimethyldodecahydronaphtho[2,1-b]furan-2-yl) furan-2(5H)-one (Intermediate 3) (0.62 kg). Total yield: 1.72 kg (1.10 kg and 0.62 kg, 65.0%).

¹H NMR (400 MHz, CDCl₃, δ): 7.28 (t, J=2.0 Hz, 1H), 4.82 (t, J=2.4 Hz, 2H), 4.70 (m, 1H), 4.29-4.24 (m, 2H), 3.46 (d, J=10.0 Hz, 1H), 3.35-3.25 (m, 1H), 2.46 (q, J=8.0 Hz, 1H), 2.21-2.17 (m, 1H), 2.05-2.01 (m, 1H), 1.82-1.68 (m, 4H), 1.57-1.40 (m, 4H), 1.25 (s, 3H), 1.13 (s, 3H), 0.97 (s, 3H), 0.91 (s, 9H), 0.1 (s, 6H).

### Step 2: Synthesis of Intermediate 4

To a solution of 3-((2S,3aR,5aS,6R,7R,9aR,9bS)-6-(((tert-butyldimethylsilyl)oxy) methyl)-7-hydroxy-3a,6,9a-trimethyldodecahydronaphtho[2,1-b]furan-2-yl) furan-2(5H)-one (Intermediate 3) (1.0 kg, 2.15 mol., 1.0 eq.) in DCM (20 L) were added Pyridine (0.255 kg, 3.22 mol., 1.5 eq.), Celite (2 kg, 2 w/w) and Pyridinium chlorochromate (0.927 kg, 4.3 mol., 2.0 eq.) at rt. The resulting mixture stirred at rt for 24 h. Progress of the reaction was monitored by TLC & LCMS. After completion of the reaction, the reaction mixture was filtered over celite bed (2.0 kg) and washed the bed with dichloromethane (5 L, 5 vol.). The filtrate mL's washed with water (10 L, 10 vol.) and saturated brine solution (10 L, 10 vol.), concentrated under reduced pressure. The residue obtained was dissolved in ethyl acetate (10 L, 10 vol.), was added charcoal (100.0 g, 10%) at rt and then heated at 50 °C for 1 h. The resulting mixture was filtered and washed with ethyl acetate (5 L, 5 vol.). Filtrate was concentrated under reduced pressure. The crude product (brown colour solid) was treated with Methanol (2 L, 2 vol.) under stirring at rt for 20 min., then cooled to 0 °C and maintained for 2 h. Filtered the product and washed with cold methanol (0.5 L, 0.5 vol.), dried under vacuum to afford (2S,3aR,5aS,6R,9aR,9bS)-6-(((tert-butyldimethylsilyl)oxy) methyl)-3a,6,9a-trimethyl-2-(2-oxo-2,5-dihydrofuran-3-yl) decahydronaphtho[2,1-b]furan-7(3aH)-one (Intermediate 4), as off white solid. Yield: 650.0 g (65.2%).

¹H NMR (400 MHz, CDCl₃, δ): 7.31 (d, J=2.0 Hz, 1H), 4.83-4.77 (m, 3H), 3.84 (d, J=10 Hz, 1H), 3.59 (d, J=10 Hz, 1H), 2.68-2.50 (m, 2H), 2.37-2.21 (m, 2H), 2.15-2.03 (m, 1H), 2.02-1.92 (m, 1H), 1.83-1.68 (m, 1H), 1.66-1.48 (m, 4H), 1.43-1.33 (m, 2H), 1.28 (s, 3H), 1.15 (s, 3H), 1.01 (s, 3H), 0.86 (s, 9H). 13C NMR (100 MHz, CDCl₃, δ): 214.1, 172.5, 143.1, 138.4, 82.7, 73.1, 70.5, 66.5, 57.2, 53.9, 53.2, 39.1, 36.1, 35.9, 35.3, 33.0, 31.6, 25.8, 22.3, 19.3, 18.2, 15.5.

### Step 3: Synthesis of Intermediate 5

(2S,3aR,5aS,6R,9aR,9bS)-6-(((tert-butyldimethylsilyl)oxy)methyl)-3a,6,9a-trimethyl-2-(2-oxo-2,5-dihydrofuran-3-yl) decahydronaphtho[2,1-b]furan-7(3aH)-one (Intermediate 4) (650.0 g, 1.4 mol., 1.0 eq.) was added lot wise to a mixture of hydroxylamine hydrochloride (341.0 g, 4.9 mol., 3.5 eq.), pyridine (1.14 L, 14.0 mol., 10.0 eq.) in Methanol (7.8 L, 12 vol.) at rt. The resulting mixture was stirred at rt for 4 h. Progress of the reaction was monitored by TLC & LCMS. After completion of the reaction, ice cold water (6.5 L) was added. The precipitated solids were filtered, and washed with heptane (2.6 L, 4 vol.). Compound co distilled with Toluene twice (2.6 L, 2×1 vol.) under reduced pressure. The crude product was treated with heptane (6.5 L, 10 vol.) under stirring at rt for 2 h. Filtered the product under vacuum and washed with heptane (1.3 L, 2 vol.), dried to afford 3-((2S,3aR,5aS,6S,9aR,9bS,E)-6-(((tert-butyldimethylsilyl)oxy)methyl)-7-(hydroxyimino)-3a,6,9a-trimethyldodecahydronaphtho[2,1-b]furan-2-yl) furan-2(5H)-one (Intermediate 5), as off white solid. (598.0 g). Crude product was taken directly for the next step without further purification.

¹H NMR (400 MHz, CDCl₃, δ): 7.28 (d, J=2 Hz, 1H), 4.81 (brs, 2H), 4.75 (t, J=9.6 Hz, 1H), 3.76 (d, J=6 Hz, 1H), 3.58 (d, J=10 Hz, 1H), 3.25-3.15 (m, 1H), 2.51-2.45 (m, 1H), 2.24-2.19 (m, 1H), 2.1-1.95 (m, 2H), 1.8-1.7 (m, 1H), 1.58-1.48 (m, 4H), 1.2 (s, 3H), 1.15 (s, 3H), 1.1 (s, 3H), 0.86 (s, 9H), 0.06 (s, 6H). 13C NMR (100 MHz, CDCl₃, δ): 172.5, 163.4, 143.1, 138.4, 82.8, 73.1, 70.5, 66.9, 57.6, 53.6, 39.1, 36.3, 35.8, 32.9, 31.6, 25.8, 22.2, 19.0, 18.2, 17.8, 15.8.

### Steps 4 and 5: Synthesis of Intermediate 6, Compound 2, and Intermediate 7

To a solution of 3-((2S,3aR,5aS,6S,9aR,9bS,E)-6-(((tert-butyldimethylsilyl)oxy)methyl)-7-(hydroxyimino)-3a,6,9a-trimethyldodecahydronaphtho[2,1-b]furan-2-yl) furan-2(5H)-one (Intermediate 5) (0.630 kg, 1.31 mol., 1.0 eq.) in Acetonitrile (12.6 L, 20 vol.) were added 4-toluenesulfonyl chloride (0.377 kg, 1.97 mol., 1.5 eq.) and potassium carbonate (0.546 kg, 3.95 mol., 3.0 eq.) at 0 °C. The reaction mixture stirred at the same temperature for a further 4 h. Progress of the reaction was monitored by TLC & LCMS. After completion of the reaction, cold water (6.5 L, 10 vol.) was added to the reaction mixture and extracted with ethyl acetate (13 L, 2×10 vol.). Organic extracts were combined and washed with brine solution (6.5 L, 10 vol.), concentrated under reduced pressure. The crude product obtained was a mixture of Intermediate 6, Compound 2, and Intermediate 7, as pale brown thick syrup (0.6 kg). The crude product obtained was dissolved in ethyl acetate (6 L, 10 vol.) was added 4 M Hydrochloric acid in 1,4-dioxane (3.14 L, 12.5 mol., 10 eq.) dropwise at 0 °C. The resulting mixture was stirred at rt for 16 h. Progress of the reaction mixture was monitored by TLC. After completion of the reaction, ice cold water (6 L, 10 vol.) was added, separated the organic layer and the aqueous layer was extracted with ethyl acetate (6 L, 10 vol.). The aqueous layer was kept aside. The combined organic layer was concentrated under reduced pressure. The product obtained was treated with ethyl acetate (1.2 L, 2 vol.) under stirring at rt for 1 h, filtered the product and washed with ethyl acetate (0.6 L, 1 vol.). Compound was dried under vacuum to afford 3-((3aS,3bS,5aR,7S,8aS,8bR)-3a,5a,8b-trimethyl-3,3a,3b,4,5,5a, 7,8,8a,8b,9,10-dodecahydrofuro[3′,2′:5,6]naphtho[2,1-c]isoxazol-7-yl) furan-2(5H)-one (Intermediate 7), as off-white solid; Yield: 80.0 g (17.6%). The aqueous layer described above was adjusted pH to 8.5-9.0 using solid sodium carbonate (2.1 kg) and extracted with 10% methanol in dichloromethane (6 L, 10 vol.). The organic layer was concentrated under reduced pressure. The crude product obtained was treated with ethyl acetate (3 L, 5 vol.) at 50 °C under stirring for 2 h. The resulting mixture was cooled to rt, filtered and the product obtained was washed with ethyl acetate (1.2 L, 2 vol.) followed by heptane (1.2 L, 2 vol.), dried under vacuum to afford (2S,3aR,5aS,6R,10aR,10bS)-6-(hydroxymethyl)-3a,6,10a-trimethyl-2-(2-oxo-2,5-dihydrofuran-3-yl) dodecahydro-8H-benzofuro[5,4-c]azepin-8-one (Compound 2), as off white solid. Yield: 142.0 g (31%).

Intermediate 7: ¹H NMR (400 MHz, DMSO-d₆, δ): 7.46 (q, J=2.0 Hz, 1H), 4.85 (t, J=2.4 Hz, 2H), 4.54-4.50 (m, 1H), 3.94 (d, J=8.4 Hz, 1H), 3.83 (d, J=8.4 Hz, 1H), 2.62-2.53 (m, 1H), 2.46-2.40 (m, 1H), 2.34-2.27 (m, 1H), 2.13-2.01 (m, 2H), 1.85-1.76 (m, 1H), 1.61 (d, J=7.2 Hz, 1H), 1.56-1.45 (m, 2H), 1.43-1.32 (m, 2H), 1.26-1.18 (m, 1H), 1.13 (s, 3H), 1.05 (s, 3H), 1.05 (s, 3H). 13C NMR (100 MHz, DMSO-d₆, δ): 172.4, 164.9, 136.3, 81.9, 76.9, 71.9, 70.6, 54.3, 52.0, 49.9, 38.8, 35.9, 35.3, 34.0, 32.2, 30.8, 25.3, 18.8, 16.9, 13.5. LC-MS: m/z 346.2 (M+H).

Compound 2: 1H NMR (400 MHz, DMSO-d₆, δ): 7.48 (d, J=2.0 Hz, 1H), 6.47 (brs, 1H), 4.86 (brs, 2H), 4.82 (t, J=5.2 Hz, 1H), 4.50 (t, J=7.6 Hz, 1H), 3.59-3.45 (m, 2H), 2.34-2.29 (m, 2H), 2.20 (q, J=7.6 Hz, 1H), 2.02-1.92 (m, 2H), 1.79-1.74 (m, 1H), 1.72-1.59 (m, 2H), 1.58-1.46 (m, 3H), 1.42-1.33 (m, 1H), 1.18 (s, 3H), 1.13 (s, 3H), 1.04 (s, 3H). 13C NMR (100 MHz, DMSO-d₆, δ): 172.2, 172.4, 146.1, 135.4, 82.2, 71.6, 70.7, 65.6, 60.2, 54.7, 50.3, 38.5, 35.7, 34.8, 32.7, 30.9, 30.9, 28.2, 20.4, 17.53. LC-MS: m/z 364.2 (M+H)

### Representative Synthesis of Compound 3 (Also Referred to Herein as Compound III)

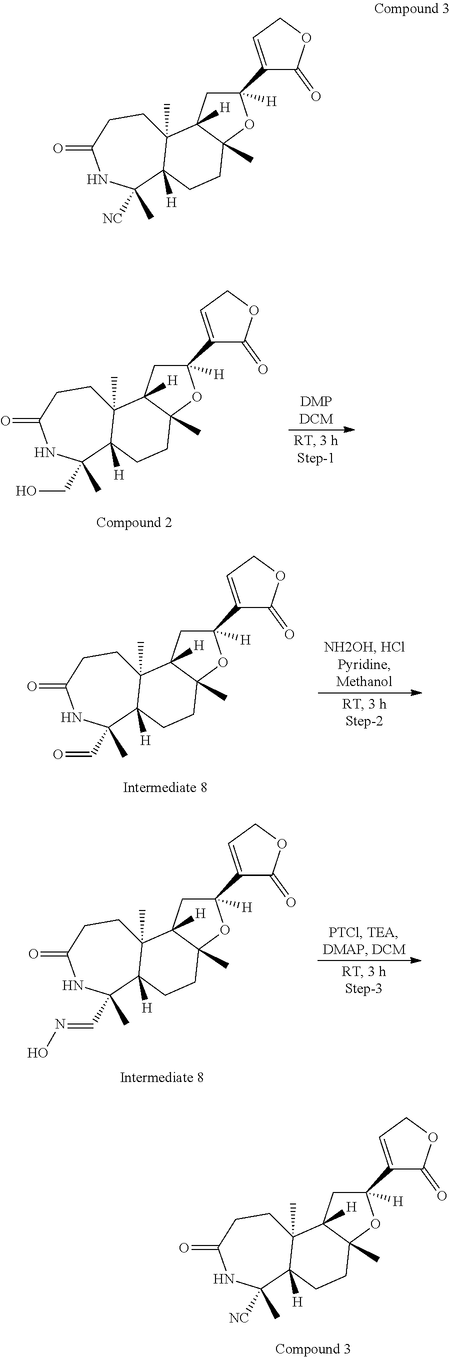

### Step 1: Synthesis of Intermediate 8

To a solution of (2S,3aR,5aS,6R,10aR,10bS)-6-(hydroxymethyl)-3a,6,10a-trimethyl-2-(2-oxo-2,5-dihydrofuran-3-yl) dodecahydro-8H-benzofuro[5,4-c]azepin-8-one (Compound 2) (30.0 g, 82.5 mmol, 1.0 eq.) in (600 mL) of DCM was added DMP (87.5 g, 206 mmol, 2.5 eq.) at rt. The resulting mixture was stirred at rt for 3 h. Progress of the reaction mixture was monitored by TLC. After completion of the reaction, dilute the reaction mixture with DCM (100 mL), wash with saturated hypo solution twice (2×100 mL), saturated NaHCO₃ solution (100 mL) and water (100 mL). The combined organic layer was dried over Na₂SO₄, filtered, and concentrated under reduced pressure. The obtained crude product was triturated with methanol (90 mL) under stirring at rt for 30 min. Filtered the product, washed with cold methanol (10 mL) and dried under reduced pressure to afford (2S,3aR,5aS,6R,10aR,10bS)-3a,6,10a-trimethyl-8-oxo-2-(2-oxo-2,5-dihydrofuran-3-yl) dodecahydro-2H-benzofuro[5,4-c]azepine-6-carbaldehyde (Intermediate 8), as white colour solid. Yield: 29.0 g (97.2%).

Intermediate 8: 1H NMR (400 MHz, CDCl₃, δ): 9.76 (s, 1H), 7.31 (q, J=2.0 Hz, 1H), 5.81 (brs, 1H), 4.83 (t, J=2.4 Hz, 2H), 4.72-4.68 (m, 1H), 2.45 (q, J=8.0 Hz, 1H), 2.40-2.35 (m, 2H), 2.32-2.25 (m, 1H), 2.15-1.85 (m, 4H), 1.78-1.72 (m, 1H), 1.7-1.6 (m, 3H), 1.43 (s, 3H), 1.2 (s, 3H), 1.05 (s, 3H). 13C (NMR 100 MHz, CDCl₃, δ): 199.0, 178.6, 172.4, 143.4, 137.7, 82.2, 72.9, 70.5, 66.2, 56.4, 55.0, 39.6, 36.4, 35.5, 33.4, 31.2, 30.5, 25.3, 20.4, 16.2.

### Step 2: Synthesis of Intermediate 9

To a solution of (2S,3aR,5aS,6R,10aR,10bS)-3a,6,10a-trimethyl-8-oxo-2-(2-oxo-2,5-dihydrofuran-3-yl) dodecahydro-2H-benzofuro[5,4-c]azepine-6-carbaldehyde (Intermediate 8) (28.0 g, 77.5 mmol, 1.0 eq.) in methanol (350 mL) was added a pre-dissolved solution of hydroxylamine hydrochloride (8.07 g, 116 mmol, 1.5 eq.) and pyridine (62.4 mL, 775 mmol, 10.0 eq.) in methanol (200 mL) drop wise at RT. The resulting mixture was stirred at RT for 3 h. Progress of the reaction mixture was monitored by TLC. After completion of the reaction, concentrated the reaction mixture under reduced pressure, ice cold water (500 mL) was added. The precipitated product was filtered and dried under vacuum to afford (E)-3a,6,10a-trimethyl-8-oxo-2-(2-oxo-2,5-dihydrofuran-3-yl) dodecahydro-2H-benzofuro[5,4-c]azepine-6-carbaldehyde oxime (Intermediate 9), as off white solid (19.0 g). Crude product was taken directly for the next step without further purification.

¹H NMR (400 MHz, CDCl₃, δ): 8.79 (d, J=2.8 Hz, 1H), 7.53 (s, 1H), 7.30 (q, J=2.0 Hz, 1H), 5.59 (brs, 1H), 4.82 (t, J=2.4 Hz, 2H), 4.68 (t, J=2.8 Hz, 1H), 2.65-2.53 (m, 1H), 2.46 (q, 7.6 Hz, 1H), 2.35 (q, J=7.6 Hz, 1H), 2.25-2.17 (m, 1H), 2.10-1.90 (m, 1H), 1.7-1.6 (m, 5H), 1.55-1.5 (m, 1H), 1.48-1.38 (m, 4H), 1.19 (s, 3H), 0.9 (s, 3H). 13C NMR (100 MHz, CDCl₃, δ): 179.7, 172.3, 151.6, 143.4, 137.7, 82.3, 72.8, 70.5, 58.7, 56.5, 54.2, 39.7, 36.6, 35.0, 33.2, 31.2, 30.8, 30.7, 20.3, 15.6.

### Step 3: Synthesis of Compound 3

To a solution of (E)-3a,6,10a-trimethyl-8-oxo-2-(2-oxo-2,5-dihydrofuran-3-yl) dodecahydro-2H-benzofuro[5,4-c]azepine-6-carbaldehyde oxime (Intermediate 9) (19.0 g, 50.5 mmol, 1.0 eq.) in DCM (600 mL) was added Et₃N (21.1 mL, 151 mmol, 3.0 eq.) and cooled the reaction mixture to 0 °C, followed by the addition of 4-methylbenzene-1-sulfonyl chloride (14.4 g, 75.7 mmol, 1.5 eq.) and DMAP (0.617 g, 5.05 mmol, 0.1 eq.). The resulting mixture was allowed to reach rt and stirred at rt for 3 h. Progress of the reaction mixture was monitored by TLC. After completion of the reaction, diluted the reaction mixture with DCM (300 mL) and washed with water (3×100 mL). The separated organic layer was dried over Na₂SO₄, filtered, and concentrated under reduced pressure. To the crude product obtained was treated with methanol (100 mL) and stirred at 0 °C for 30 min. Filtered the product and washed with cold methanol (10 mL), dried under vacuum to afford (2S,3aR,5aS,6R,10aR,10bS)-3a,6,10a-trimethyl-8-oxo-2-(2-oxo-2,5-dihydrofuran-3-yl) dodecahydro-2H-benzofuro[5,4-c]azepine-6-carbonitrile (Compound 3), as off white solid. Yield: 17.0 g (93.97%).

¹H NMR (400 MHz, CDCl₃, δ): 7.33 (q, 2.0 Hz, 1H), 5.95 (brs, 1H), 4.83-4.82 (m, 2H), 4.70-4.66 (m, 1H), 2.98-2.91 (m, 1H), 2.47-2.41 (m, 2H), 2.20-2.15 (m, 1H), 2.05-1.85 (m, 2H), 1.80-1.63 (m, 7H), 1.42-1.38 (m, 5H), 1.24 (s, 3H). 13C NMR (100 MHz, CDCl₃, δ): 177.7, 172.3, 144.0, 136.5, 120.5, 82.1, 72.6, 70.5, 58.0, 52.1, 51.5, 40.0, 36.9, 33.2, 32.8, 31.1, 30.3, 29.6, 20.9, 14.0. LC-MS: m/z 359.2 (M+H).

### Representative Synthesis of Reductive Amination Products (Compounds 4 to 14)

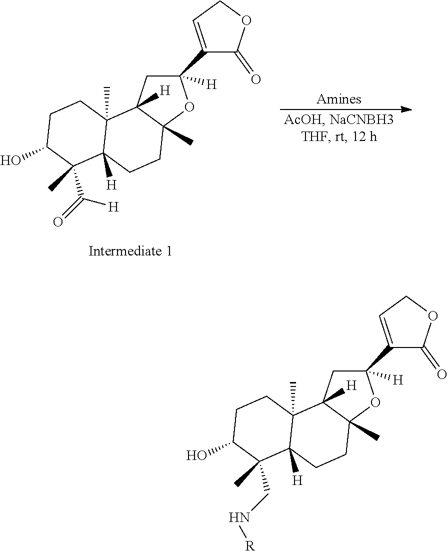

### Representative Synthesis of Compound 4

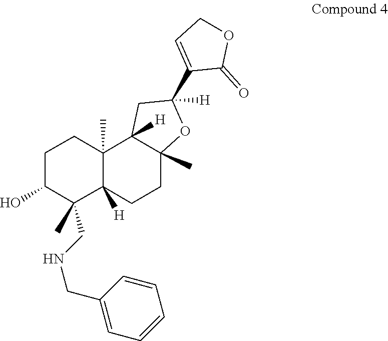

To a stirred solution of (2S,3aR,5aS,6S,7R,9aR,9bS)-7-hydroxy-3a,6,9a-trimethyl-2-(2-oxo-2,5-dihydrofuran-3-yl) dodecahydronaphtho[2,1-b]furan-6-carbaldehyde (Intermediate 1) (1 g, 2.87 mmol, 1 eq.) in THF (10 mL), Benzylamine (533 μL, 4.8 mmol, 1.7 eq.), acetic acid (catalytical amount, 82 μL) were added at rt. After 30 min. NaCNBH3 (361 mg, 5.74 mmol, 2 eq.) was added, the resulting reaction mixture was stirred at rt for 12 h. Progress of the reaction was monitored by TLC, diluted the reaction mixture with water (50 mL) and extracted with EtOAc (3×40 mL). The combined organic layer was dried over Na₂SO₄, filtered and concentrated under reduced pressure. The crude product obtained was purified by column chromatography by using (100-200 silica gel mesh) MeOH in DCM as eluent to afford 3-((2S,3aR,5aS,6R,7R,9aR,9bS)-6-((benzylamino)methyl)-7-hydroxy-3a,6,9a-trimethyldodecahydronaphtho[2,1-b]furan-2-yl) furan-2(5H)-one (Compound 4), as off white solid. Yield: 430 mg (34%).

¹H NMR (400 MHz, CDCl3, δ): 7.36-7.27 (m, 6H), 4.80 (d, J=4.4 Hz, 2H), 4.67 (t, J=8.0 Hz, 1H), 3.76 (q, J=12.8 Hz, 2H), 3.31 (dd, J=11.6, 3.2, 1H), 3.11 (d, J=12.0 Hz, 1H), 2.72 (dd, J=12.0, 0.8, Hz, 1H), 2.41 (q, J=8.0 Hz, 1H), 2.15 (d, J=12.4, 5.2 Hz, 1H), 2.05-1.96 (m, 1H), 1.75-1.67 (m, 2H), 1.53-1.45 (m, 5H), 1.24 (bs, 3H), 1.10 (bs, 3H), 1.04 (d, J=3.6 Hz, 1H), 0.99 (d, J=3.6 Hz, 1H), 0.96 (d, J=3.2 Hz, 1H), 0.90 (s, 3H). 13C NMR (100 MHz, CDCl3, δ): 175.1, 172.6, 161.2, 158.5, 143.2, 138.3, 137.3, 128.6, 127.5, 82.72, 80.4, 73.1, 70.8, 58.33, 54.3, 53.4, 50.7, 46.3, 40.5, 39.2, 35.8, 32.8, 31.5, 27.8, 23.1, 18.4, 16.3.

### Representative Synthesis of Compound 5

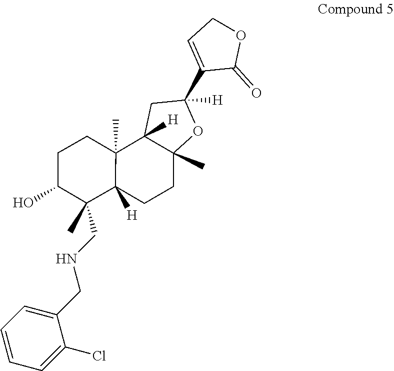

To a stirred solution of (2S,3aR,5aS,6S,7R,9aR,9bS)-7-hydroxy-3a,6,9a-trimethyl-2-(2-oxo-2,5-dihydrofuran-3-yl) dodecahydronaphtho[2,1-b]furan-6-carbaldehyde (Intermediate 1) (1 g, 2.87 mmol, 1 eq.) in THF (10 mL), 2-chloro-benzylamine (417 μL, 3.44 mmol, 1.2 eq.) and acetic acid (catalytical amount) (49 mg), were added at rt. After 30 min. was added NaCNBH3 (361 mg, 5.74 mmol, 2 eq.) at rt, the resulting reaction mixture was stirred at rt for 12 h. Progress of the reaction was monitored by TLC. After completion of the reaction, diluted the reaction mixture with water (30 mL) and extracted with EtOAc (3×40 mL). The combined organic layer was dried over Na₂SO₄, filtered and concentrated under reduced pressure. The crude product obtained was purified by column chromatography by using (100-200 silica gel mesh) MeOH in DCM as eluent to afford 3-((2S,3aR,5aS,6R,7R,9aR,9bS)-6-(((2-chlorobenzyl)amino)methyl)-7-hydroxy-3a,6,9a-trimethyldodecahydronaphtho[2,1-b]furan-2-yl) furan-2(5H)-one (Compound 5), as off white solid. Yield: 50 mg (3%).

¹H NMR (400 MHz, CDCl3, δ): 7.38 (d, J=4 Hz, 2H), 7.28-7.25 (m, 3H), 4.81 (s, 2H), 4.69 (t, J=7.2 Hz, 1H), 3.91 (q, J=13.6, 2H), 3.33 (dd, J=11.6, 3.6 Hz, 2H), 3.12 (d, J=12 Hz, 1H), 2.72 (d, J=10.8 Hz, 1H), 2.43 (q, J=8.0, 1H), 2.17 (dt, J=0 3.6 Hz, Hz, 1H), 2.05-2.01 (m, 1H), 1.78-1.69 (m, 2H), 1.54-1.46 (m, 5H), 1.26 (s, 3H), 1.11 (s, 3H), 1.00 (d, J=4.4 Hz, 1H) 0.94 (s, 3H), 0.90 (dd, J=10.2, 3.2 Hz, 2H). 13C NMR (100 MHz, CDCl3, δ): 172.5, 143.1, 138.2, 135.0, 133.9, 130.8, 129.6, 129.1, 127.02, 82.6, 73.0, 70.4, 58.2, 53.1, 51.4, 50.4, 50.4, 39.0, 35.9, 35.7, 32.7, 31.45, 27.6, 23.0, 18.3, 16.2.

### Representative Synthesis of Compound 6

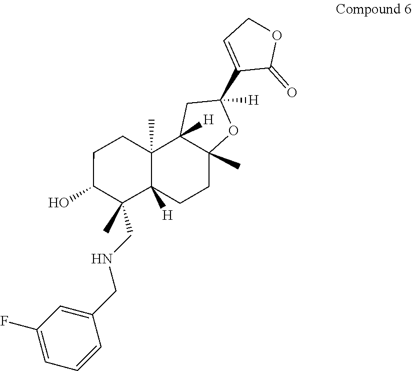

To a solution of Intermediate 1 (1 g, 2.87 mmol, 1 eq.) in THF (10 mL) were added 3-Fluorobenzylamine (0.4 mL, 2.87 mmol, 1.2 eq.), acetic acid (172 mg) at rt. After 4 h, were added methanol (5 mL), and NaCNBH₃ (361 mg, 5.74 mmol, 2 eq.). The resulting mixture was stirred at rt for 12 h. Progress of the reaction was monitored by TLC. After completion of the reaction, diluted the reaction mixture with water (15 mL) and extracted with EtOAc (2×20 mL). The combined organic layer was dried over Na₂SO₄, filtered and concentrated under reduced pressure. The crude product obtained was purified by column chromatography by using (60-120 silica gel mesh) MeOH in DCM as eluent to afford the desired product Compound 6, as off white solid. Yield: 210 mg (15%).

¹H NMR (400 MHz, CDCl3, δ): 7.32-7.27 (m, 1H), 7.27 (s, 1H), 7.06 (d, J=8 Hz, 1H), 7.00-6.94 (m, 2H), 4.80 (t, J=2.8 Hz, 2H), 4.68 (t, J=7.6 Hz, 1H), 3.73 (d, J=2.8 Hz, 2H), 3.30 (dd, J=10.4, 4.0 Hz, 1H), 3.09 (d, J=12.0 Hz, 1H), 2.70 (d, J=10.4 Hz, 1H), 2.42 (q, J=8.0 Hz, 1H), 2.16 (dt, J=14.4, 3.2 Hz, 1H), 2.04-1.96 (m, 1H), 1.79-1.68 (m, 2H), 1.61-1.45 (m, 6H), 1.24 (s, 3H), 1.21 (d, J=5.2 Hz, 1H), 1.16 (d, J=3.2 Hz, 1H), 1.10 (s, 3H), 1.00 (td, J=12.8, 2.4 Hz, 1H), 0.93 (s, 3H), 0.89 (t, J=4.8 Hz, 1H), 0.86 (d, J=4.0 Hz, 1H), 13C NMR (100 MHz, CDCl3, δ): 177.2, 172.4, 164.0, 161.5, 143.0, 141.3, 138.3, 130.0, 123.7, 115.1, 114.4, 82.6, 80.4, 73.0, 70.4, 58.2, 54.0, 53.2, 50.9, 46.6, 40.6, 39.6, 36.0, 35.8, 32.9, 31.4, 27.8, 23.0, 18.3, 16.4.

### Representative Synthesis of Compound 7

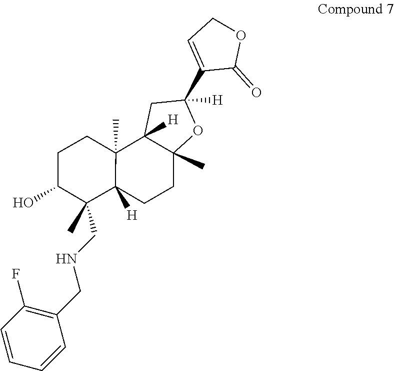

To a solution of Intermediate 1 (1 g, 2.87 mmol, 1 eq.) in THF (10 mL) were added 2-Fluorobenzylamine (431 mg, 3.44 mmol, 1.2 eq.), acetic acid (172 mg) at rt. After 4 h were added methanol (5 mL) and NaBH₄ (271 mg, 5.74 mmol, 2 eq.). The resulting mixture was stirred at rt for a further 12 h. Progress of the reaction was monitored by TLC. After completion of the reaction, diluted the reaction mixture with water (15 mL), and extracted with DCM (3×20 mL). The combined organic layer was dried over Na₂SO₄, filtered and concentrated under reduced pressure. The crude product obtained was purified by column chromatography by using (60-120 silica gel mesh) MeOH in DCM as eluent to afford Compound 7 as off white solid, Yield: 300 mg (21%).

¹H NMR (400 MHz, CDCl3, δ): 7.30-7.27 (m, 2H), 7.244 (d, J=3.2 Hz, 1H), 7.12 (dd, J=12.0 Hz, 7.6 Hz, 1H), 7.09 (m, 1H), 4.80 (t, J=2.8 Hz, 2H), 4.68 (t, J=2.8 Hz, 1H), 3.76 (s, 2H), 3.29 (dt, J=8.4 Hz, 3.2 Hz, 1H), 3.08 (d, J=12 Hz, 1H), 2.69 (dd, J=12 Hz, 1H), 2.43 (q, J=8.0 Hz, 1H), 2.15 (dt, J=14.4 Hz, 4.4 Hz, 1H), 2.03-1.97 (m, 1H), 1.77-1.70 (m, 3H), 1.53 (t, J=5.6 Hz, 2H), 1.48-1.41 (m, 3H), 1.25 (d, J=3.2 Hz, 1H), 1.22 (s, 3H), 1.09 (s, 3H), 1.00 (d, J=0 Hz, 1H), 0.95 (s, 3H), 0.89 (q, J=4.8 Hz, 1H). 13C NMR (100 MHz, CDCl3, δ): 172.5, 162.3, 159.9, 143.0, 138.4, 130.5, 129.2, 125.8, 124.2, 115.4, 82.6, 80.4, 73.1, 70.4, 58.3, 53.3, 50.8, 47.8, 40.6, 37.2, 36.1, 32.8, 31.5, 29.6, 27.9, 23.0, 18.40, 16.2.

### Representative Synthesis of Compound 8

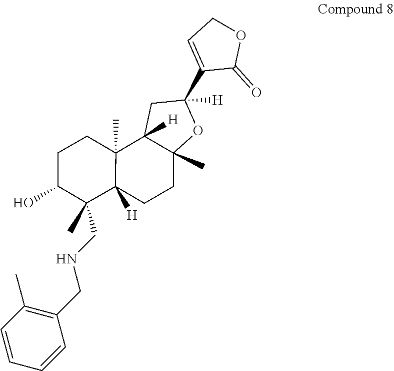

To a solution of Intermediate 1 (1 g, 2.87 mmol, 1 eq.) in THF (10 mL) were added 2-methyl benzyl amine (417 mg, 3.44 mmol, 1.2 eq.), acetic acid (172 mg) at rt. After 4 h were added methanol (5 mL), and NaBH₄ (217 mg, 5.74 mmol, 2 eq.). The resulting mixture was stirred at rt for a further 12 h. Progress of the reaction was monitored by TLC. After completion of the reaction, diluted the reaction mixture diluted with water (10 mL), then extracted to DCM (2×10 mL). The combined organic layer was dried over Na₂SO₄, filtered, and concentrated under reduced pressure. The crude product obtained was purified by column chromatography by using (60-120 silica gel mesh) MeOH in DCM as eluent to afford Compound 8, as off white solid, Yield: 300 mg (21%).

¹H NMR (400 MHz, CDCl3, δ): 7.27 (t, J=2.0 Hz, 1H), 7.21-7.16 (m, 4H), 4.80 (t, J=7.6, 1H), 3.72 (s, 2H), 3.28 (dd, J=11.6 Hz, 2.4 Hz, 1H), 3.14 (d, J=12.0 Hz, 1H), 2.75 (d, J=11.6 Hz, 1H), 2.43 (q, J=7.6 Hz, 1H), 2.34 (s, 3H), 2.18 (dd, J=10.8 Hz, 3.6 Hz, 1H), 2.04-1.96 (m, 1H), 1.77-1.69 (m, 3H), 1.55-1.45 (m, 6H), 1.25 (t, J=2.0, 1H), 1.23 (s, 3H), 1.10 (s, 3H), 1.01 (dd, J=13.2 Hz, 3.2 Hz, 1H), 0.96 (s, 3H). 13C NMR (100 MHz, CDCl3, δ): 172.5, 143.0, 138.3, 136.5, 136.4, 130.4, 128.9, 127.5, 125.9, 82.6, 80.3, 73.1, 70.4, 58.2, 53.2, 52.2, 51.2, 40.6, 39.1, 36.1, 35.8, 32.8, 31.5, 27.8, 23.0, 18.9, 18.4, 16.3.

### Representative Synthesis of Compound 9

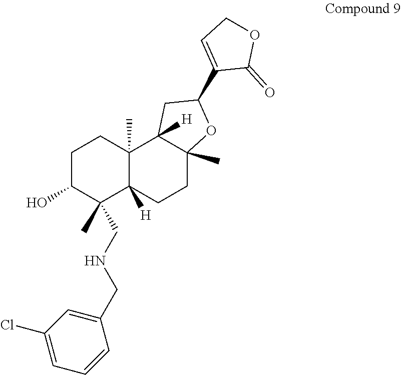

To a solution of Intermediate 1 (1 g, 2.87 mmol, 1 eq.) in THF (10 mL) were added 3-Chlorobenzylamine (610 mg, 4.34 mmol, 1.5 eq.), acetic acid (172 mg) at rt. After 30 min. was added NaBH3CN (361 mg, 5.74 mmol, 2 eq.). The resulting mixture was stirred at rt for a further 12 h. Progress of the reaction was monitored by TLC. After completion of the reaction, diluted the reaction mixture with water (10 mL), and extracted with DCM (2×10 mL). The combined organic layer was dried over Na₂SO₄, filtered, and concentrated under reduced pressure. The crude product obtained was purified by column chromatography by using (60-120 silica gel mesh) MeOH in DCM as eluent to afford Compound 9, as off white solid. Yield: 340 mg (24%).

¹H NMR (400 MHz, CDCl3, δ): 7.28 (m, 2H), 7.24 (d, J=2.0 Hz, 1H), 7.20-7.16 (m, 1H), 4.80 (t, J=2.8 Hz, 2H), 4.70 (t, J=7.6 Hz, 1H), 3.71 (s, 2H), 3.30 (dd, J=10.4 Hz, 2.8 Hz, 1H), 3.08 (d, J=12.0 Hz, 1H), 2.70 (dd, J=12.0 Hz, 1.2 Hz, 1H), 2.43 (q, J=8.0 Hz, 1H), 2.19 (dd, J=6.4 Hz, 3.2 Hz, 1H), 2.04-1.96 (m, 1H), 1.79-1.69 (m, 3H), 1.61-1.41 (m, 6H), 1.29 (d, J=2.8 Hz, 1H), 1.24 (s, 3H), 1.10 (s, 3H), 1.02 (dd, J=2.8 Hz, 1H), 0.97 (s, 3H), 0.93 (d, J=6.4 Hz, 1H). 13C NMR (100 MHz, CDCl3, δ): 172.5, 143.0, 140.8, 138.3, 134.3, 129.8, 128.3, 127.5, 126.3, 82.6, 80.4, 73.1, 58.2, 54.2, 53.2, 51.0, 40.6, 39.2, 36.1, 35.8, 32.8, 31.5, 27.8, 23.08, 18.4, 16.3.

### Representative Synthesis of Compound 10

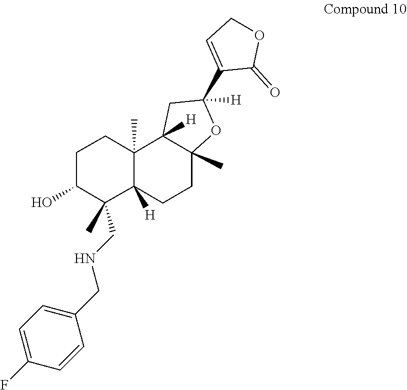

To a solution of Intermediate 1 (1 g, 2.87 mmol, 1 eq.) in THF (10 mL) were added 4-Fluorobenzylamine (431 mg, 3.44 mmol, 1.2 eq.), acetic acid (0.164 mL, 2.87 mmol, 1 eq.) at rt. After 30 min. was added NaBH₃CN (361 mg, 5.74 mmol, 2 eq.). The resulting mixture was stirred at rt for a further 12 h. Progress of the reaction was monitored by TLC. After completion of the reaction, diluted the reaction mixture with water (10 mL), and extracted with DCM (2×10 mL). The combined organic layer was dried over Na₂SO₄, filtered, and concentrated under reduced pressure. The crude product obtained was purified by column chromatography by using (60-120 silica gel mesh) MeOH in DCM as eluent to afford Compound 10, as off white solid. Yield: 310 mg (23%).

¹H NMR (400 MHz, CDCl3, δ): 7.26-7.22 (m, 3H), 7.00 (t, J=8.8 Hz, 2H), 4.79 (t, J=2 Hz, 2H), 4.67 (t, J=8.0 Hz, 1H), 3.68 (q, J=12.8 Hz, 2H), 3.28 (dd, J=11.2, 2.4, 1H), 3.07 (d, J=12.0 Hz, 1H), 2.68 (d, J=12.0 Hz, 1H), 2.41 (q, J=8.0 Hz, 1H), 2.16 (dd, J=10.4, 3.2 Hz, 1H), 2.00-1.97 (m, 1H), 1.75-1.87 (m, 2H), 1.52-1.45 (m, 5H), 1.22 (s, 3H), 1.09 (s, 3H), 1.04 (d, J=3.6 Hz, 1H), 0.99 (d, J=3.6 Hz, 1H), 0.96 (d, J=3.2 Hz, 1H), 0.91 (s, 3H), 0.87 (d, J=3.6 Hz, 1H). 13C NMR (100 MHz, CDCl3, δ): 172.5, 163.3, 160.9, 143.1, 138.4, 134.6, 134.5, 129.9, 115.4, 115.2, 82.7, 80.5, 73.1, 70.5, 58.3, 53.3, 50.8, 40.66 39.25 36.15 35.87 32.87 31.57 29.69 27.95 23.12 18.47 16.35.

### Representative Synthesis of Compound 11

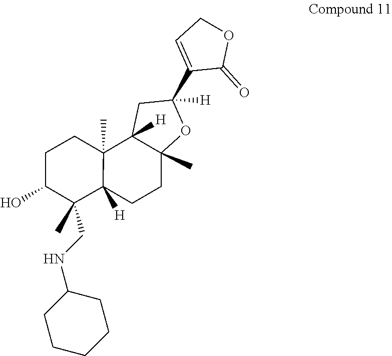

To a solution of Intermediate 1 (250 mg, 0.717 mmol, 1 eq.) in THF (8 mL) were added cyclohexylamine (0.123 mL, 1.08 mmol, 1.5 eq.), acetic acid (catalytical amount) (0.02 mL) at rt. After 1 h was added NaCNBH₃ (90.2 mg, 1.43 mmol, 2 eq.). The resulting mixture was stirred at rt for 5 h. Progress of the reaction was monitored by TLC. After completion of the reaction, water (25 mL) was added to the reaction mixture and extracted with EtOAc (3×20 mL). The combined organic layer was dried over Na₂SO₄, filtered and concentrated under reduced pressure. The crude product obtained was purified by column chromatography by using (100-200 silica gel mesh) Methanol in DCM as eluent to afford Compound 11, as white solid. Yield: 70 mg (22%).

¹H NMR (400 MHz, CDCl₃, δ): 7.29 (t, J=4.0 Hz, 1H), 4.83 (d, 2H), 4.71 (d, J=15.2 Hz, 1H), 3.34-3.31 (dd, 1H), 3.14 (d, 1H), 2.72 (d, J=11.6 Hz, 1H), 2.72 (d, 1H), 2.48-2.38 (m, 2H), 2.21-2.17 (t, 1H), 2.03-2.00 (m, 2H), 1.95-1.48 (m, 10H), 1.27-1.25 (d, 4H), 1.12 (s, 3H), 1.01 (s, 3H), 0.96-0.89 (m, 1H). ¹³C NMR (100 MHz, CDCl₃, δ): 172.5, 143.1, 138.4, 82.7, 80.5, 73.1, 70.5, 58.4, 57.1, 53.3, 48.2, 40.3, 39.3, 36.1, 35.8, 32.2, 33.0, 32.8, 31.6, 28.1, 25.9, 24.8, 24.8, 23.2, 18.4, 16.5.

### Representative Synthesis of Compound 12

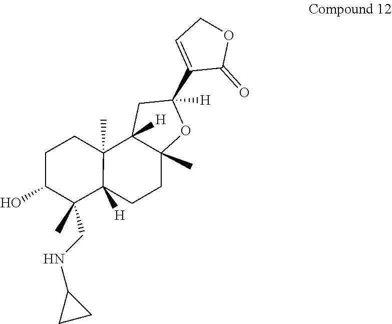

To a solution of Intermediate 1 (200 mg, 0.574 mmol, 1 eq.) in THF (8 mL) were added cyclopropylamine (0.0597 mL, 0.861 mmol, 1.5 eq.) and acetic acid (catalytical amount) (0.01 mL) was added at rt. After 1 h was added NaCNBH₃ (72.1 mg, 1.15 mmol, 2 eq.). The resulting mixture was stirred at rt for 5 h. Progress of the reaction was monitored by TLC. After completion of the reaction, water (25 mL) was added to the reaction mixture and extracted with EtOAc (3×30 mL). The combined organic layer was dried over Na₂SO₄, filtered and concentrated under reduced pressure. The crude product obtained was purified by column chromatography by using (100-200 silica gel mesh) Methanol in DCM as eluent to afford Compound 12, as white solid. Yield: 70 mg (31%).

¹H NMR (400 MHz, DMSO, 8): 7.46 (d, J=1.2 Hz, 1H), 4.86 (s, 2H), 4.51 (t, J=15.2 Hz, 1H), 3.35-3.23 (m, 3H), 2.88 (d, 1H), 2.60-2.50 (brs, 1H), 2.25-2.20 (m, 1H), 2.08-1.97 (m, 2H), 1.67-1.59 (m, 3H), 1.55-1.41 (m, 3H), 1.14 (s, 3H), 1.06 (s, 3H), 1.03-0.99 (m, 2H), 0.88 (s, 4H), 0.72-0.66 (m, 3H). ¹³C NMR (100 MHz, DMSO, 8): 172.9, 146.2, 136.5, 82.4, 78.4, 72.5, 71.2, 57.6, 52.2, 50.8, 38.4, 35.9, 35.5, 32.6, 31.7, 31.4, 27.1, 23.1, 18.3, 16.1.

### Representative Synthesis of Compound 13

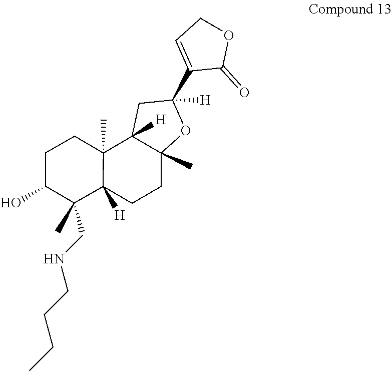

To a solution of Intermediate 1 (300 mg, 0.861 mmol, 1 eq.) in THF (8 mL) were added butan-1-amine (0.111 mL, 1.12 mmol, 1.3 eq.) and acetic acid (catalytical amount) (0.03 mL) at rt. After 1 h was added NaCNBH₃ (108 mg, 1.72 mmol, 2 eq.). The resulting mixture was stirred at rt for 5 h. Progress of the reaction was monitored by TLC. After completion of the reaction, water (25 mL) was added to the reaction mixture and extracted with EtOAc (3×25 mL). The combined organic layer was dried over Na₂SO₄, filtered and concentrated under reduced pressure. The crude product obtained was purified by recrystallization using MeOH:DCM (9:1) (5 mL). Filtered the solids and dried under vacuum to afford Compound 13, as white solid. Yield: 110 mg (31%).

¹H NMR (400 MHz, DMSO, 6): 7.47 (s, 1H), 4.68 (d, 2H0, 3.19 (q, 1H), 2.93 (q, 2H0, 2.81 (d, 1H), 2.25-2.20 (q, 1H), 2.08-1.98 (m, 2H), 1.64-1.47 (m, 8H), 1.39 (br, s, 2H), 1.35-1.28 (m, 2H), 1.17 (s, 3H), 1.06-1.07 (s, 4H), 0.9-0.8 (q, 6H). 13C NMR (100 MHz, DMSO, 8): 172.9, 146.2, 136.5, 82.4, 78.2, 72.5. 71.1, 57.5, 52.0, 49.2, 48.1, 38.2, 35.8, 35.4, 32.5, 31.4, 27.5, 26.8. 22.8, 19.7, 18.2, 16.1, 13.9.

### Representative Synthesis of Compound 14

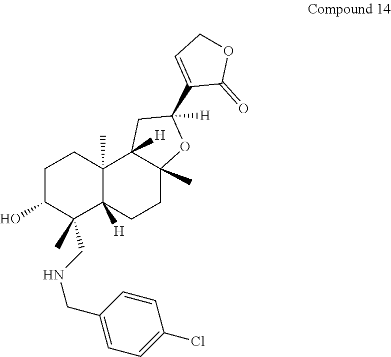

To a solution of Intermediate 1 (100 mg, 0.287 mmol, 1 eq.) in THF (3 mL) were added 1-(4-chlorophenyl) methanamine (48.8 mg, 0.344 mmol, 1.2 eq.), acetic acid (catalytical amount) (0.01 mL) at rt. After 30 min. was added NaCNBH₃ (36.1 mg, 0.574 mmol, 2 eq.). The resulting mixture was stirred at rt for further 12 h. Progress of the reaction was monitored by TLC. After completion of the reaction, diluted the reaction mixture with water (15 mL) and extracted with EtOAc (3×20 mL). The combined organic layer was dried over Na₂SO₄, filtered and concentrated under reduced pressure. The crude product obtained was purified by column chromatography by using (100-200 silica gel mesh) MeOH in DCM as eluent to afford Compound 14, as off white solid. Yield: 70 mg (51%).

¹H NMR (400 MHz, CDCl₃, δ): 7.35-7.28 (m, 4H), 4.05 (d, 1H), 3.85 (d, 1H), 3.44 (dd, 1H), 3.19 (d, 1H), 2.88 (d, 1H), 2.41 (q, 1H), 2.19 (t, 1H), 2.06-1.98 (m, 2H), 1.71 (d, 2H), 1.56-1.38 (m, 6H), 1.36-1.27 (s, 6H), 1.25-1.18 (m, 4H), 1.16-1.02 (m, 2H), 0.99-0.81 (m, 4H). ¹³C NMR (100 MHz, CDCl₃, δ): 172.5, 143.2, 138.2, 134.9, 131.6, 130.8, 129.2, 82.5, 79.5, 73.1, 70.5, 58.1, 52.8, 52.0, 49.5, 40.3, 38.7, 35.9, 35.6, 32.8, 31.4, 29.6, 26.7, 22.6, 18.2, 16.1.

### Representative Synthesis of Compound 15

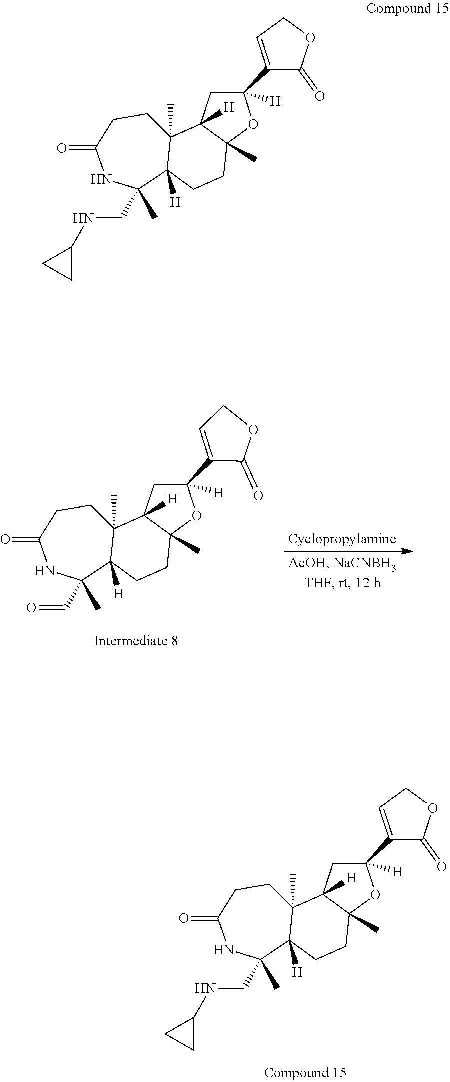

To a solution of Intermediate 8 (250 mg, 0.692 mmol, 1 eq.) in THF (8 mL) were added cyclopropylamine (0.072 mL, 1.04 mmol, 1.5 eq.) and acetic acid (catalytical amount) (0.1 mL) at rt. After 1 h, was added NaCNBH₃ (86.9 mg, 1.38 mmol, 2 eq.). The resulting mixture was stirred at rt for 12 h. Progress of the reaction was monitored by TLC. After completion of the reaction, water (25 mL) was added to the reaction mixture and extracted with EtOAc (3×25 mL). The combined organic layer was dried over Na₂SO₄, filtered, and concentrated under reduced pressure. The crude product obtained was purified by flash column chromatography by using MeOH in DCM as eluent, to afford Compound 15, as white colour solid. Yield: 80 mg (28%).

¹H NMR (400 MHz, CDCl₃, δ): 7.36-7.28 (q, 1H), 5.92 (s, 1H), 4.82 (s, 2H), 4.69 (t, 3H), 3.08 (d, 1H), 2.83 (d, 1H), 2.49-2.42 (m, 4H), 2.16-1.99 (m, 2H), 1.79-1.51 (m, 9H), 1.27-1.19 (m, 10H), 0.42 (d, 2H), 0.31 (t, 2H). 13C NMR (100 MHz, CDCl₃, δ): 176.8, 172.5, 143.6, 137.6, 82.6, 72.7, 70.5, 60.0, 56.3, 5b4.9, 51.5, 39.3, 36.8, 35.1, 33.5, 31.3, 31.2, 30.8, 29.5, 20.9, 18.4, 6.8, 6.6.

### Representative Synthesis of Reductive Amination Products (Compounds 16 and 17)

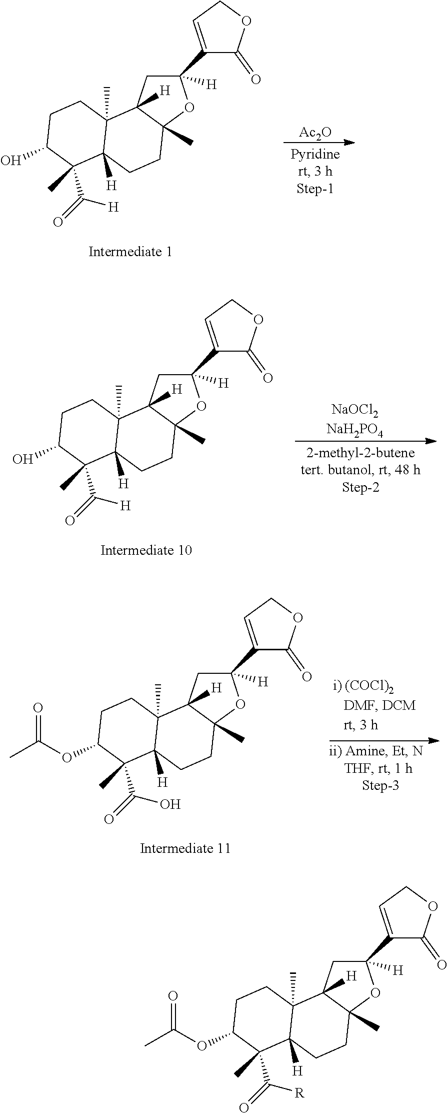

### Representative Synthesis of Compound 16

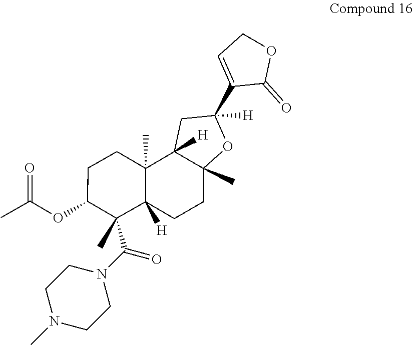

### Step 1: Synthesis of Intermediate 10

To a solution of Intermediate 1 (6.0 g, 17.2 mmol, 1 eq.) in pyridine (10 mL) at rt was added acetic anhydride (6 mL) dropwise at rt. The resulting mixture was stirred at rt for 3 h. Progress of the reaction was monitored by TLC. After completion of the reaction, ice cold water (100 mL) was added and the precipitated solids were filtered, washed with water (100 mL) and dried under vacuum to afford Intermediate 10, as off white solid. Yield: 5.6 g (83.29%).

¹H NMR (400 MHz, CDCl₃, δ): 10.10 (s, 1H), 7.28 (t, J=3.2 Hz, 1H), 4.82 (s, 2H), 4.73-4.69 (q, 2H), 2.50-2.44 (q, 1H), 2.24-2.19 (t, 1H), 2.07-2.04 (t, 4H), 2.01-1.96 (m, 1H), 1.90-1.86 (dd, 1H), 1.75-1.70 (t, 1H), 1.63-1.61 (d, 1H), 1.57-1.39 (m, 2H), 1.24-1.21 (d, 2H), 1.30 (s, 3H), 0.93 (s, 3H). ¹³C NMR (100 MHz, CDCl₃, δ): 203.9, 172.5, 170.4, 143.2, 138.2, 82.4, 78.5, 73.1, 70.5, 56.6, 54.4, 51.5, 38.3, 35.7, 35.6, 33.1, 31.3, 23.8, 2123, 21.0, 18.3, 16.9.

### Step 2: Synthesis of Intermediate 11

To a solution of Intermediate 10 5.5 g, 14.1 mmol, 1 eq.) in tert-butanol (180 mL), 2-methyl-2-butene (20.9 mL, 197 mmol, 14 eq.) and water (140 mL) were added NaOCl₂ (5.1 g, 56.3 mmol, 4 eq.) and NaH₂PO₄ (6.65 g, 56.3 mmol, 4 eq.) at rt. The resulting mixture was stirred at rt for 48 h. Progress of the reaction was monitored by TLC. After completion of the reaction, the reaction mixture was diluted with EtOAc (200 mL). The organic layer was separated, washed with water (3×80 mL). The combined organic layer was dried over Na₂SO₄, filtered and concentrated under reduced pressure. The crude product obtained was purified by column chromatography by using (100-200 silica gel mesh) EtOAc in Hexane as eluent to afford Intermediate 11 as white solid. Yield: 3.7 g (64.62%).

¹H NMR (400 MHz, CDCl₃, δ): 7.29 (t, J=6.8 Hz, 1H), 4.79 (d, 3H), 4.59-4.55 (dd, 1H), 2.52-2.45 (m, 1H), 2.42-2.35 (q, 1H), 2.28-2.24 (d, 1H), 2.15-1.14 (t, 1H), 2.09 (s, 3H), 1.87-1.80 (m, 2H), 1.77-1.68 (m, 2H), 1.56-1.51 (m, 2H), 1.30 (s, 3H), 1.16-1.14 (d, 2H), 1.12 (s, 3H), 1.01 (s, 3H). ¹³C NMR (100 MHz, CDCl₃, δ): 177.5, 172.3, 171.0, 143.4, 138.1, 82.9, 79.2, 73.2, 70.5, 57.2, 53.3, 47.7, 39.1, 36.3, 36.1, 32.6, 31.3, 24.2, 23.8, 21.3, 19.6, 13.9.

### Step 3: Synthesis of Compound 16

To a solution of Intermediate 11 (2 g, 4.92 mmol, 1 eq.) in DCM (15 mL) was added oxalyl chloride (1.27 mL, 14.8 mmol, 3 eq.), followed by the addition of catalytical amount of DMF (0.1 mL) at rt and the resulting mixture was stirred at rt for further 3 h. Progress of the reaction was monitored by TLC. After completion of the reaction, the reaction mixture was concentrated under reduced pressure. The residue obtained was dissolved in dry THF (12 mL) and was added to a mixture of 1-methylpiperazine (0.819 mL, 7.38 mmol, 1.5 eq.) and triethylamine (2.06 mL, 14.08 mmol, 3 eq.) in dry THF (3 mL) at 0 °C and the resulting mixture was stirred at rt for 1 h. Progress of the reaction was monitored by TLC. After completion of the reaction, was added water (80 mL) and extracted with EtOAc (4×40 mL). The combined organic layer was dried over Na₂SO₄, filtered and concentrated under reduced pressure. The crude product obtained was purified by column chromatography over silica gel (100-200 mesh) by using a mixture of MeOH in DCM as eluent to afford Compound 16, as yellow solid. Yield: 1.5 g (62.39%).

¹H NMR (400 MHz, CDCl₃, δ): 7.28 (s, 1H), 4.82 (s, 2H), 4.74 (t, J=14.8 Hz, 1H), 3.65 (s, 4H), 3.22 (s, 1H), 2.82-2.74 (m, 1H), 2.53-2.48 (m, 1H), 2.42-2.38 (m, 4H), 2.30 (s, 3H), 2.08 (s, 5H), 1.86 (d, 1H), 1.73-1.70 (q, 2H), 1.66-1.57 (m, 3H), 1.40 (s, 3H), 1.20 (s, 3H), 1.13 (s, 3H), 1.08 (s, 3H). ¹³C NMR (100 MHz, CDCl₃, δ): 173.0, 172.4, 171.2, 143.1, 138.3, 85.2, 82.2, 73.3, 70.4, 58.0, 55.4, 53.6, 50.1, 46.0, 45.7, 39.6, 36.5, 35.9, 33.0, 31.5, 26.9, 25.9, 24.8, 21.5, 20.2, 13.9.

### Representative Synthesis of Compound 17

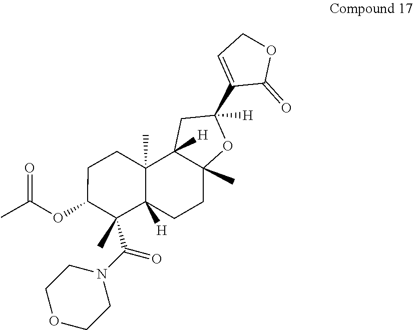

To a solution of Intermediate 11 (400 mg, 0.984 mmol, 1 eq.) in DCM (8 mL) was added oxalyl chloride (0.253 mL, 2.95 mmol, 3.0 eq.), followed by the addition of catalytical amount of DMF (0.02 mL) at rt and the resulting mixture was stirred at rt for further 3 h. Progress of the reaction was monitored by TLC. After completion of the reaction, the reaction mixture was concentrated under reduced pressure. The residue obtained was dissolved in dry THF (6 mL) and was added to a mixture of morpholine (0.127 mL, 1.48 mmol, 1.5 eq.) and triethylamine (0.411 mL, 2.95 mmol, 3.0 eq.) in dry THF (2 mL) at 0 °C, and the resulting mixture was stirred at rt for 1 h. Progress of the reaction was monitored by TLC. After completion of the reaction, was added water (80 mL) and extracted with EtOAc (3×30 mL). The combined organic layer was dried over Na₂SO₄, filtered, and concentrated under reduced pressure. The crude product obtained was purified by column chromatography over silica gel (100-200 mesh) by using a mixture of MeOH in DCM as eluent to afford Compound 17, as yellow solid. Yield: 220 mg (47.01%).

¹H NMR (400 MHz, CDCl₃, δ): 7.28 (s, 1H), 4.82 (t, J=4.0 Hz, 2H), 4.74 (t, 1H), 4.59-4.55 (dd, 1H), 3.70-3.65 (m, 7H), 2.75-2.11 (m, 3H), 2.09 (s, 3H), 2.07-2.03 (m, 1H), 1.90-1.85 (m, 1H), 1.77-1.56 (m, 9H), 1.40 (s, 3H), 1.16 (s, 3H), 1.11 (s, 3H). ¹³C NMR (100 MHz, CDCl₃, δ): 173.2, 172.4, 171.1, 143.1, 138.3, 85.1, 82.1, 73.3, 70.4, 67.1, 58.0, 53.6, 50.1, 46.8, 39.6, 36.5, 35.9, 33.0, 31.5, 25.8, 24.8, 21.4, 20.2, 13.9.

### Representative Synthesis of Compound 18

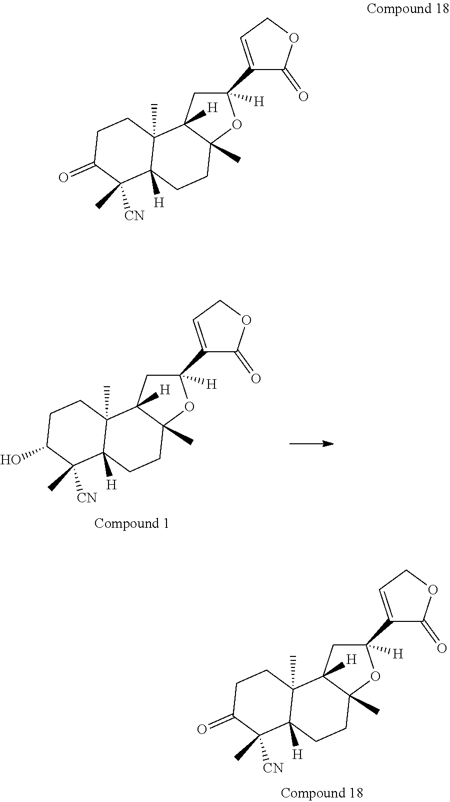

To a solution of (2S,3aR,5aS,6R,7R,9aR,9bS)-7-hydroxy-3a,6,9a-trimethyl-2-(2-oxo-2,5-dihydrofuran-3-yl)-dodecahydronaphtho[2,1-b]furan-6-carbonitrile Compound 1 (0.7 g, 2.03 mmol, 1 eq.) in DCM (15 mL) was added DMP (2.15 g, 5.07 mmol, 2.5 eq.) at rt. The mixture was stirred at rt for 3 h. Progress of the reaction was monitored by TLC. After completion of the reaction, the reaction mixture was diluted with DCM (80 mL) and washed with saturated NaHCO₃ solution (50 mL), saturated hypo solution (2×80 mL) and brine solution (80 mL). The combined organic layer was dried over Na₂SO₄, filtered, and concentrated under reduced pressure. The crude product obtained was purified by flash column chromatography by using (100-200 silica gel mesh) EtOAc in Hexane as eluent to afford the desired product (2S,3aR,5aS,6R,9aR,9bS)-3a,6,9a-trimethyl-7-oxo-2-(2-oxo-2,5-dihydrofuran-3-yl)-dodecahydronaphtho[2,1-b]furan-6-carbonitrile (Compound 18), as white solid. Yield: 510 mg (73.28%).

¹H NMR (400 MHz, CDCl₃, δ): 7.33 (d, J=1.6 Hz, 1H), 4.81 (t, J=15.6 Hz, 2H), 2.99-2.98 (dd, 1H), 2.50-2.10 (m, 2H), 2.47-2.01 (m, 4H), 1.73-1.59 (m, 6H), 1.51 (d, 5H), 1.44-1.27 (m, 3H), 1.16 (s, 3H). ¹³C NMR (100 MHz, CDCl₃, δ): 202.4, 143.5, 137.9, 119.7, 82.3, 73.2, 70.5, 56.4, 53.9, 46.5, 40.4, 36.0, 35.2, 34.5, 32.7, 31.3, 21.0, 20.6, 14.0.

### Representative Synthesis of Compound 19

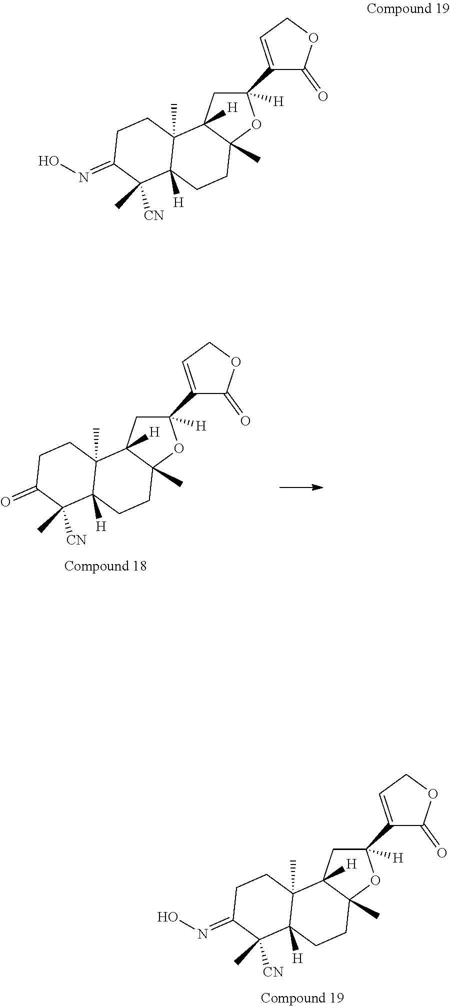

A mixture of hydroxylamine hydrochloride (1.98 g, 28.5 mmol, 3.5 eq.) and pyridine (6.57 mL, 81.5 mmol, 10.0 eq.) in methanol (25 mL) was added drop wise to a solution of (2S,3aR,5aS,6R,9aR,9bS)-3a,6,9a-trimethyl-7-oxo-2-(2-oxo-2,5-dihydrofuran-3-yl)-dodecahydronaphtho[2,1-b]furan-6-carbonitrile (Compound 18) (2.8 g, 8.15 mmol, 1.0 eq.) in methanol (25 mL) at RT. The resulting mixture was stirred at RT for 3 h. Progress of the reaction was monitored by TLC. After completion of the reaction, the reaction mixture was concentrated under reduced pressure and the obtained crude was washed with ice cold water (200 mL). The precipitated solids were filtered and dried under vacuum to afford as crude product (2S,3aR,5aS,6S,7E,9aR,9bS)-7-(hydroxyimino)-3a,6,9a-trimethyl-2-(2-oxo-2,5-dihydrofuran-3-yl)-dodecahydronaphtho[2,1-b]furan-6-carbonitrile Compound 19, as off white solid. Crude product was taken directly for the next step without further purification. Yield: 2.0 g (68.44%).

¹H NMR (400 MHz, CDCl₃, δ): 7.41 (s, 1H), 7.31 (q, 1H), 4.813-4.82 (q, 2H), 4.79-4.74 (m, 1H), 3.32-3.26 (m, 1H), 2.54-2.32 (q, 1H), 2.31-2.02 (m, 5H), 1.94-1.86 (m, 2H), 1.78-1.71 (m, 5H), 1.56 (s, 3H), 1.54-1.21 (m, 5H). ¹³C NMR (100 MHz, CDCl₃, δ): 172.5, 157.3, 143.5, 137.8, 121.5, 82.4, 73.1, 70.5, 56.5, 52.4, 39.8, 38.4, 36.0, 34.4, 32.6, 31.2, 22.8, 20.1, 18.2, 13.8.

### Representative Synthesis of Compound 20

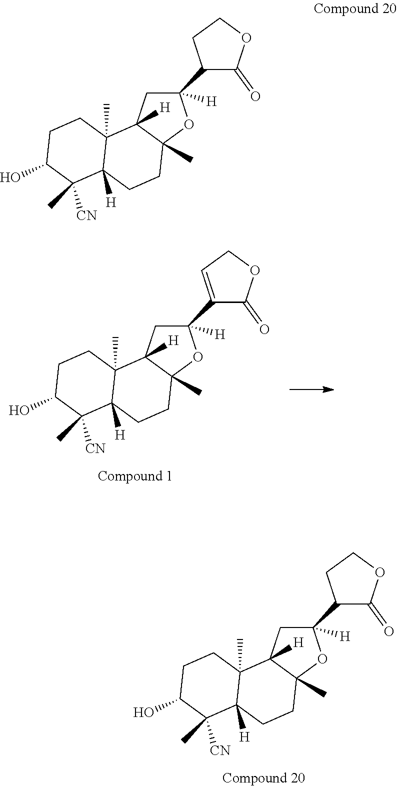

To a solution of (2S,3aR,5aS,6R,7R,9aR,9bS)-7-hydroxy-3a,6,9a-trimethyl-2-(2-oxo-2,5-dihydrofuran-3-yl)-dodecahydronaphtho[2,1-b]furan-6-carbonitrile (Compound 1) (0.5 g, 1.45 mmol, 1 eq.) in a 1:1 mixture of EtOH & THF (20 mL) at rt was added Pd/C (200 mg). The resulting mixture was stirred at rt under hydrogen gas pressure at rt for 12 h. The reaction mixture was monitored by TLC. After completion of the reaction, the reaction mixture was filtered through celite bed and washed the bed with EtOH (20 mL). The combined filtrate was concentrated under reduced pressure. The crude product obtained was purified by column chromatography by using (100-200 silica gel mesh) EtOAC in Hexane as eluent to afford (2S,3aR,5aS,6R,7R,9aR,9bS)-7-hydroxy-3a,6,9a-trimethyl-2-(2-oxooxolan-3-yl)-dodecahydronaphtho[2,1-b]furan-6-carbonitrile (Compound 20). Yield: 300 mg (59.65%).

¹H NMR (400 MHz, DMSO, 8): 5.42 (d, 1H), 4.31-4.23 (m, 1H), 4.2-4.1 (m, 1H), 4.0-3.9 (m, 1H), 3.32 (s, 3H), 3.11 (m, 1H), 2.81-2.70 (m, 1H), 2.30-2.21 (m, 1H), 2.2-2.1 (m, 1H), 2.2-1.9 (m, 3H), 1.75-1.56 (m, 5H), 1.15-1.39 (m, 4H), 1.40-1.30 (s, 4H), 1.28-1.20 (m, 2H), 1.20-1.15 (m, 1H), 1.14 (s, 3H), 1.14-1.10 (m, 1H), 1.05 (s, 3H), 1.00 (m, 1H). 13C NMR (100 MHz, DMSO, 8): 177.3, 122.7, 81.6, 76.1, 74.2. 70.5, 66.6, 57.3, 51.9, 46.1, 44.5, 43.2, 41.9, 38.4, 35.7, 32.5, 31.7, 30.9, 30.3, 29.5, 27.9. 24.4, 22.8, 19.2, 14.2.

### Representative Synthesis of Compound 21

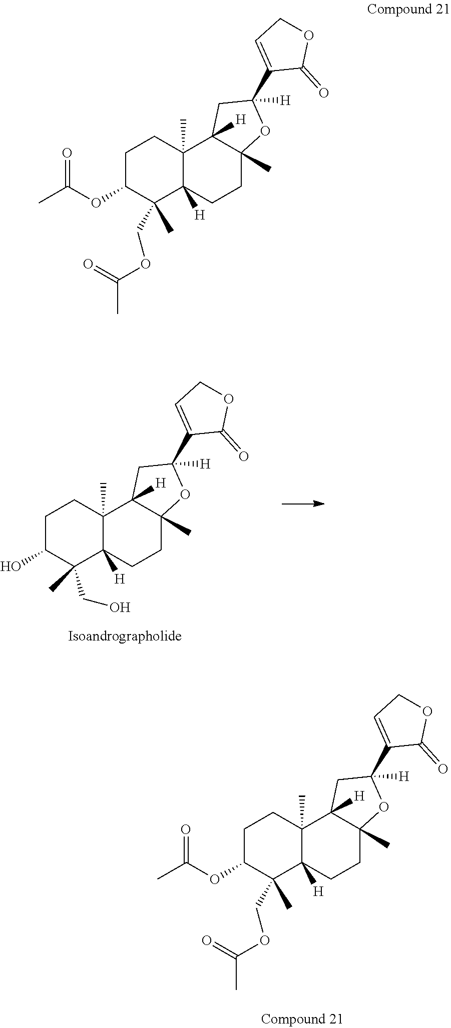

To a solution of 3-[(2S,3aR,5aS,6R,7R,9aR,9bS)-7-hydroxy-6-(hydroxymethyl)-3a,6,9a-trimethyl-dodecahydronaphtho[2,1-b]furan-2-yl]-2,5-dihydrofuran-2-one (Isoandrographolide) (1 g, 2.85 mmol, 1 eq.) in pyridine (2 mL) was added acetic anhydride (4 mL) dropwise at rt. The resulting mixture was kept at rt for further 16 h. The reaction was monitored by TLC. After completion of the reaction, the reaction mixture was poured into crushed ice (100 g), filtered the solids and dried under vacuum to afford the crude (850 mg). The crude product obtained was purified by column chromatography over (100-200 silica gel mesh) EtOAc in Hexane as eluent to afford [(2S,3aR,5aS,6R,7R,9aR,9bS)-7-(acetyloxy)-3a,6,9a-trimethyl-2-(2-oxo-2,5-dihydrofuran-3-yl)-dodecahydronaphtho[2,1-b]furan-6-yl]methyl acetate (Compound 21), as white solid. Yield: 0.34 g (27.42%).

¹H NMR (400 MHz, CDCl₃): δ 7.28 (t, J=2.0 Hz, 1H), 4.82 (s, 2H), 4.73 (t, J=9.2 Hz, 1H), 4.59 (q, J=5.6 Hz, 1H), 4.41 (d, J=11.6 Hz, 1H), 4.20 (d, J=11.6 Hz, 1H), 2.47 (q, J=8.0 Hz, 1H), 2.22 (dd, J=11.2 Hz, 3.2 Hz, 1H), 2.05 (s, 6H), 1.82 (d, J=3.2 Hz, 1H), 1.79 (d, J=3.2 Hz, 1H), 1.71-1.62 (m, 5H), 1.58-1.42 (m, 2H), 1.28 (d, J=13.6 Hz, 1H), 1.12 (s, 3H), 1.10 (d, J=4.4 Hz, 1H), 1.06 (s, 3H), 1.03 (s, 3H). 13C NMR (100 MHz, CDCl₃): δ 172.5, 170.8, 170.5, 143.1, 138.4, 82.6, 80.12, 73.2, 70.5, 65.0, 57.8, 52.9, 40.9, 39.0, 36.4, 35.7, 32.8, 31.4, 23.5, 23.0, 21.1, 21.0, 18.8, 15.8.

### Representative Synthesis of Compound 22

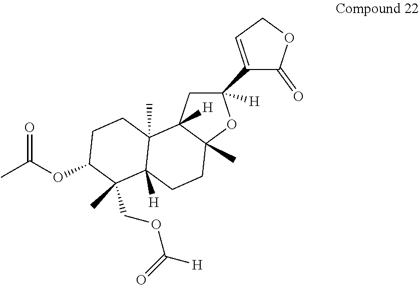

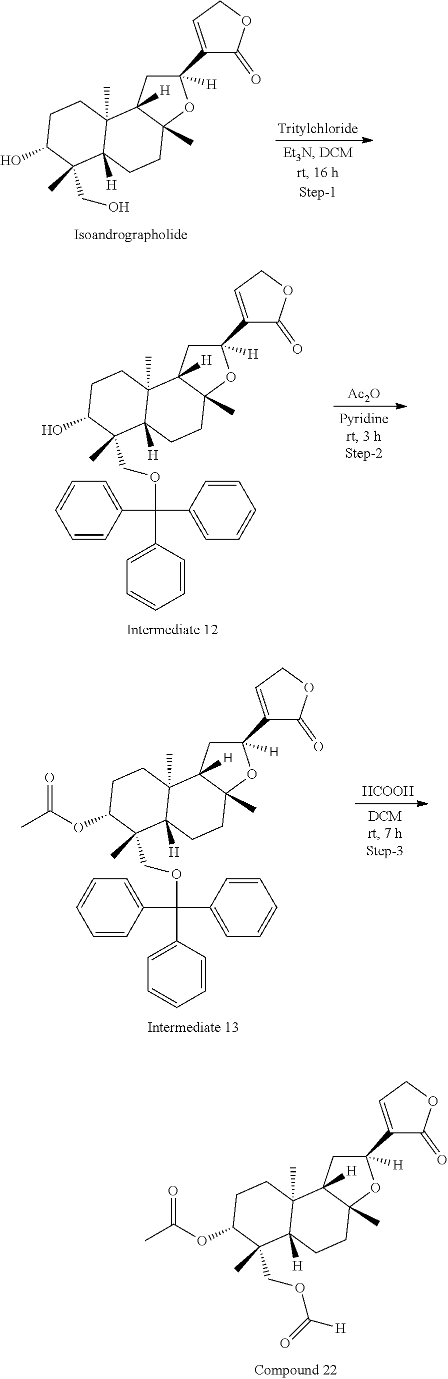

### Step 1: Synthesis of Intermediate 12

To a solution of 3-((2S,3aR,5aS,6R,7R,9aR,9bS)-7-hydroxy-6-(hydroxymethyl)-3a,6,9a-trimethyldodecahydronaphtho[2,1-b]furan-2-yl) furan-2(5H)-one (Isoandrographolide) (9.5 g, 27.1 mmol, 1 eq.) in DCM (150 mL) were added triethylamine (7.56 mL, 54.2 mmol, 2 eq.) and trityl chloride (8.31 g, 29.8 mmol, 1.1 eq.) portion wise at rt. The resulting mixture was kept at rt for further 16 h. Progress of the reaction mixture was monitored by TLC. After completion of the reaction, the reaction mixture was diluted with DCM (150 mL) and washed with water (3×100 mL). Organic layer was dried over Na₂SO₄, filtered and concentrated under reduced pressure. The crude product obtained was purified by column chromatography over silica gel (100-200 mesh) using mixtures of ethyl acetate and hexanes as eluent to afford 3-((2S,3aR,5aS,6R,7R,9aR,9bS)-7-hydroxy-3a,6,9a-trimethyl-6-((trityloxy)methyl) dodecahydronaphtho[2,1-b]furan-2-yl) furan-2(5H)-one (Intermediate 12), as white solid. Yield: 1.2 g (7.47%).

¹H NMR (400 MHz, CDCl₃): δ 7.47 (t, J=7.2 Hz, 5H), 7.34 (t, J=7.2 Hz, 5H), 7.27 (t, J=4.8 Hz, 5H), 7.24 (t, J=2.0 Hz, 1H), 4.79 (t, J=2.0 Hz, 1H), 4.57 (t, J=7.6 Hz, 1H), 4.14 (q, J=7.2 Hz, 1H), 3.47 (d, J=9.6 Hz, 1H), 3.20 (dd, J=22.0, 9.2 Hz, 1H), 2.31 (dd, J=13.6, 7.6 Hz, 8.0 Hz, 2H), 2.21 (dd, J=10.0, 5.2 Hz, 2H), 1.96-1.88 (m, 2H), 1.75-1.67 (m, 2H), 1.55 (d, J=11.2 Hz, 1H), 1.53 (s, 3H), 1.46 (t, J=8.0 Hz, 2H), 1.28 (t, J=7.2 Hz, 2H), 1.08 (s, 3H), 1.04-0.90 (m, 3H). 13C NMR (100 MHz, CDCl₃): δ 172.5, 143.4, 143.0, 138.3, 128.5, 128.0, 127.2, 87.7, 82.6, 80.6, 73.0, 70.4, 64.8, 60.4, 52.7, 42.4, 39.3, 35.8, 35.6, 33.0, 31.5, 27.4, 23.3, 18.2, 16.1, 14.2.

### Step 2: Synthesis of Intermediate 13

To a solution of 3-((2S,3aR,5aS,6R,7R,9aR,9bS)-7-hydroxy-3a,6,9a-trimethyl-6-((trityloxy)methyl) dodecahydronaphtho[2,1-b]furan-2-yl) furan-2(5H)-one (Intermediate 12) (350 mg, 0.590 mmol, 1 eq.) in pyridine (3 mL) was added acetic solvent (90.4 mg, 0.886 mmol, 1.5 eq.) at rt. The resulting mixture was stirred at rt for further 3 h. Progress of the reaction mixture was monitored by TLC. After completion of the reaction, the reaction mixture was diluted with DCM (50 mL) and washed with water (3×20 mL). Organic layer was dried over Na₂SO₄, filtered and concentrated under reduced pressure. The crude product obtained was purified by column chromatography over silica gel (100-200 mesh) using mixtures of ethyl acetate and hexanes as eluent to afford (2S,3aR,5aS,6R,7R,9aR,9bS)-3a,6,9a-trimethyl-2-(2-oxo-2,5-dihydrofuran-3-yl)-6-((trityloxy)methyl) dodecahydronaphtho[2,1-b]furan-7-yl acetate (Intermediate 13), as off-white solid. Yield: 0.3 g (80.04%).

¹H NMR (400 MHz, CDCl₃): δ 7.52-7.49 (m, 5H), 7.32-7.24 (m, 10H), 7.23 (t, J=2.4 Hz, 1H), 4.81 (t, J=2.0 Hz, 2H), 4.62 (t, J=7.6 Hz, 1H), 4.56 (dd, J=12 Hz, 4.0 Hz, 12.0 Hz, 1H), 3.42 (d, J=9.6 Hz, 1H), 3.15 (d, J=9.6 Hz, 1H), 2.36 (q, J=9.0 Hz, 1H), 2.10 (dt, J=13.2 Hz, 4.4 Hz, 1H), 2.04 (s, 3H), 2.00-1.93 (m, 2H), 1.67 (dt, J=13.6 Hz, 3.6 Hz, 1H), 1.64 (dd, J=13.6 Hz, 3.6 Hz, 1H), 1.50 (d, J=7.6 Hz, 2H), 1.46-1.39 (m, 2H), 1.28 (s, 3H), 1.18 (s, 3H), 1.10 (s, 3H), 0.92-0.85 (m, 2H), 0.61 (s, 3H). 13C NMR (100 MHz, CDCl₃): δ 172.5, 170.8, 144.2, 143.0, 138.4, 128.8, 127.6, 126.8, 87.0, 82.7, 80.6, 73.1, 70.5, 62.6, 58.1, 52.8, 42.2, 39.2, 36.1, 35.6, 32.9, 31.5, 29.7, 23.6, 23.3, 21.3, 18.9, 15.5.

### Step 3: Synthesis of Compound 22

To a solution of (2S,3aR,5aS,6R,7R,9aR,9bS)-3a,6,9a-trimethyl-2-(2-oxo-2,5-dihydrofuran-3-yl)-6-((trityloxy)methyl) dodecahydronaphtho[2,1-b]furan-7-yl acetate (Intermediate 13) (280 mg, 0.441 mmol, 1 eq.) in DCM (5 mL) was added formic acid (5 mL) drop wise at rt. The resulting mixture was stirred at rt for further 7 h. Progress of the reaction mixture was monitored by TLC. After completion of the reaction, basified the reaction mixture with saturated NaHCO₃ (15 mL) solution. Separated the organic layer and the aqueous layer was extracted with DCM (2×20 mL). The combined organic layer was dried over Na₂SO₄, filtered and concentrated under reduced pressure. The crude product obtained was purified by column chromatography over silica gel (100-200 mesh) using mixtures of ethyl acetate and hexanes as eluent to afford (2S,3aR,5aS,6R,7R,9aR,9bS)-6-((formyloxy)methyl)-3a,6,9a-trimethyl-2-(2-oxo-2,5-dihydrofuran-3-yl) dodecahydronaphtho[2,1-b]furan-7-yl acetate (Compound 22), as off-white solid. Yield: 0.14 g (75.48%).

¹H NMR (400 MHz, CDCl₃): δ 8.08 (d, J=8.0 Hz, 1H), 4.82 (s, 2H), 4.72 (t, J=7.6 Hz, 1H), 4.62-4.54 (m, 2H), 4.40 (d, J=11.6 Hz, 1H), 4.23 (t, J=11.6 Hz, 1H), 2.46 (q, J=8.0 Hz, 1H), 2.22 (d, 14.4 Hz, 1H), 2.09 (t, J=4.4 Hz, 2H), 2.04 (s, 3H), 1.82-1.51 (m, 8H), 1.27 (d, 7.6 Hz, 1H), 1.12 (s, 3H), 1.08 (s, 3H), 1.03 (s, 3H). 13C NMR (100 MHz, CDCl₃): δ 172.5, 170.5, 161.0, 143.1, 138.4, 82.5, 79.9, 73.2, 70.5, 64.2, 57.8, 52.8, 40.9, 38.9, 36.2, 35.7, 32.8, 31.4, 23.5, 22.9, 21.1, 18.7, 15.8.

### Representative Synthesis of Compound 23

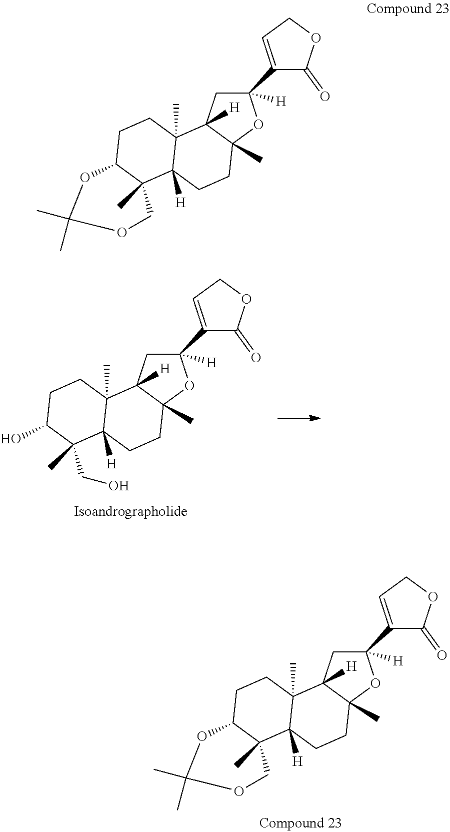

To a solution of (3-[(2S,3aR,5aS,6R,7R,9aR,9bS)-7-hydroxy-6-(hydroxymethyl)-3a,6,9a-trimethyl-dodecahydronaphtho[2,1-b]furan-2-yl]-2,5-dihydrofuran-2-one) (Isoandrographolide) (500 mg, 1.43 mmol, 1 eq.) in 2,2-dimethoxypropane (2.5 mL) was added catalytic amount of camphorsulfonic acid (16.6 mg, 0.0713 mmol, 0.05 eq.) at rt. The resulting mixture was kept at rt for further 5 h. The reaction was monitored by TLC. After completion of the reaction, the reaction mixture was concentrated under reduced pressure and the residue was diluted with EtOAc (30 mL) and washed with saturated NaHCO₃ solution (15 mL) and water (15 mL). The combined organic layer was dried over Na₂SO₄, filtered and concentrated under reduced pressure to afford the crude. The obtained crude product was purified by column chromatography by using (100-200 silica gel mesh) EtOAc:Hexane as eluent to afford (3-[(1S,2R,7R,10R,11S,13S,15R)-2,5,5,10,15-pentamethyl-4,6,14-trioxatetracyclo[8.7.0.0^{2,7}.0^{11, 15}]heptadecan-13-yl]-2,5-dihydrofuran-2-one) (Compound 23), as white solid. Yield: 0.33 g (59.23%)

¹H NMR (400 MHz, CDCl₃): δ 7.29 (t, J=1.6 Hz, 1H), 4.82 (d, J=1.6 Hz, 2H), 4.74 (t, J=9.2 Hz, 1H), 4.10 (d, J=12 Hz, 1H), 3.48 (dd, J=10.4, 4.0 Hz, 1H), 3.27 (d, J=12.0 Hz, 1H), 2.50 (q, J=8.0 Hz, 1H), 2.21 (dt, J=13.2, 4.4 Hz, 2H), 2.08-1.99 (m, 2H), 1.59 (d, 7.6 Hz, 1H), 1.54 (t, J=8.4 Hz, 1H), 1.48 (t, J=8.0 Hz, 2H), 1.46 (s, 3H), 1.39 (s, 3H), 1.25 (s, 3H), 1.17 (s, 3H), 1.12 (s, 3H), 1.07-0.95 (m, 3H). 13C NMR (100 MHz, CDCl₃): δ 172.5, 143.1, 138.5, 98.7, 82.8, 77.6, 73.1, 70.5, 63.7, 57.9, 50.5, 37.1, 37.0, 36.0, 35.1, 32.8, 31.6, 27.7, 26.6, 25.9, 25.1, 17.5, 17.2.

### Representative Synthesis of Compound 24

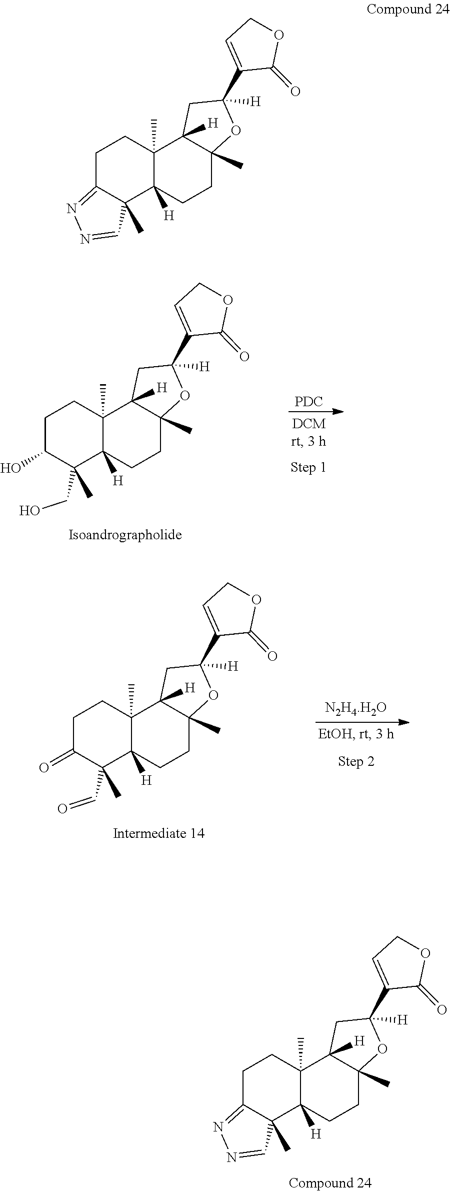

### Step 1: Synthesis of Intermediate 14

To a solution of 3-{(1R,2S,4S,6R,9S,10R,11R)-11-hydroxy-10-(hydroxymethyl)-1,6,10-trimethyl-5-oxatricyclo[7.4.0.0^{2,6}]tridec-4-yl}-2(5H)-furanone (Isoandrographolide) (500 mg, 1.43 mmol, 1 eq.) in DCM (10 mL) was added PCC (615 mg, 2.85 mmol, 2 eq.) at rt. The resulting mixture was stirred at rt for further 3 h. The reaction was monitored by TLC. After completion of the reaction, the reaction mixture was concentrated under reduced pressure. The crude product obtained was purified by column chromatography by using (100-200 silica gel mesh) EtOAc in Hexanes as eluent, to afford (((1R,2S,4S,6R,9S,10R)-1,6,10-trimethyl-11-oxo-4-(2-oxo-3-furyl)-5-oxatricyclo[7.4.0.0^{2,6}]tridecane-10-carbaldehyde) (Intermediate 14) as off white solid. Yield: 140 mg, (28.33%).

¹H NMR (400 MHz, CDCl₃) δ 9.70 (s, 1H), 7.30 (d, J=1.6 Hz, 1H), 4.83 (d, J=6.0 Hz, 2H), 2.67 (td, J=14.8, 8.8 Hz, 1H), 2.54 (q, J=8.0 Hz, 1H), 2.42 (dt, J=14.2, 2.8 Hz, 1H), 2.33 (dt, J=14.2, 2.8, 1H), 2.18-2.13 (m, 1H), 2.11-2.03 (m, 1H), 1.96 (td, J=12.8, 4.4 Hz, 1H), 1.80 (dt, J=13.2, 2.8 Hz, 2H), 1.64 (d, J=7.6 Hz, 1H), 1.58 (q, J=7.6 Hz, 1H), 1.50 (dd, J=12.8, 2.0 Hz, 1H), 1.38 (td, J=18.4, 4.4 Hz, 1H), 1.31 (s, 3H), 1.17 (s, 3H), 1.15 (s, 3H). 13C NMR (100 MHz, CDCl₃) δ 208.5, 201.3, 172.4, 143.2, 138.2, 82.3, 73.2, 70.5, 63.3, 55.7, 55.3, 39.6, 36.2, 36.1, 35.9, 32.9, 31.1, 19.2, 17.2, 14.8.

### Step 2: Synthesis of Compound 24

To a solution of ((2S,3aR,5aS,6R,9aR,9bS)-3a,6,9a-trimethyl-7-oxo-2-(2-oxo-2,5-dihydrofuran-3-yl)-dodecahydronaphtho[2,1-b]furan-6-carbaldehyde) (Intermediate 14) (100 mg, 0.289 mmol, 1 eq.) in Ethanol (5 mL) was added hydrazine hydrate (21.7 mg, 0.433 mmol, 1.5 eq.) at rt. The resulting mixture was stirred at rt for further 3 h. The reaction was monitored by TLC. After completion of the reaction, the reaction mixture was diluted with water (20 mL) and extracted with DCM (2×30 mL). The combined organic layer was dried over Na₂SO₄, filtered and concentrated under reduced pressure. The crude product obtained was purified by column chromatography by using (100-200 silica gel mesh) MeOH in DCM as eluent to afford 3-[(1S,2R,9R,10S,12S,14R)-2,9,14-trimethyl-13-oxa-4,5-diazatetracyclo[7.7.0.0^{2,6}.0^{10,14}]hexadeca-3,5-dien-12-yl]-2,5-dihydrofuran-2-one (Compound 24), as white solid. Yield: 50 mg, (50.58%).

¹H NMR (400 MHz, CDCl₃): δ 8.05 (s, 1H), 4.82 (d, J=6.0 Hz, 2H), 4.68 (t, J=8.4 Hz, 1H), 3.08-2.99 (m, 1H), 2.82 (q, J=10.4 Hz, 1H), 2.48 (q, J=8.4 Hz, 1H), 2.10 (dd, J=8.8 Hz, 2.8 Hz, 1H), 2.04-1.98 (m, 3H), 1.88-1.80 (m, 1H), 1.7 (dd, J=12.4 Hz, 2.0 Hz, 2H), 1.63-1.52 (m, 3H), 1.31 (s, 3H), 1.13 (s, 3H), 0.59 (s, 3H). 13C NMR (100 MHz, CDCl₃): δ 172.4, 170.9, 143.2, 138.3, 82.4, 70.5, 64.4, 54.8, 54.7, 37.1, 36.5, 35.5, 33.1, 31.6, 23.7, 22.7, 20.7, 13.5.

### Representative Synthesis of Compound 25

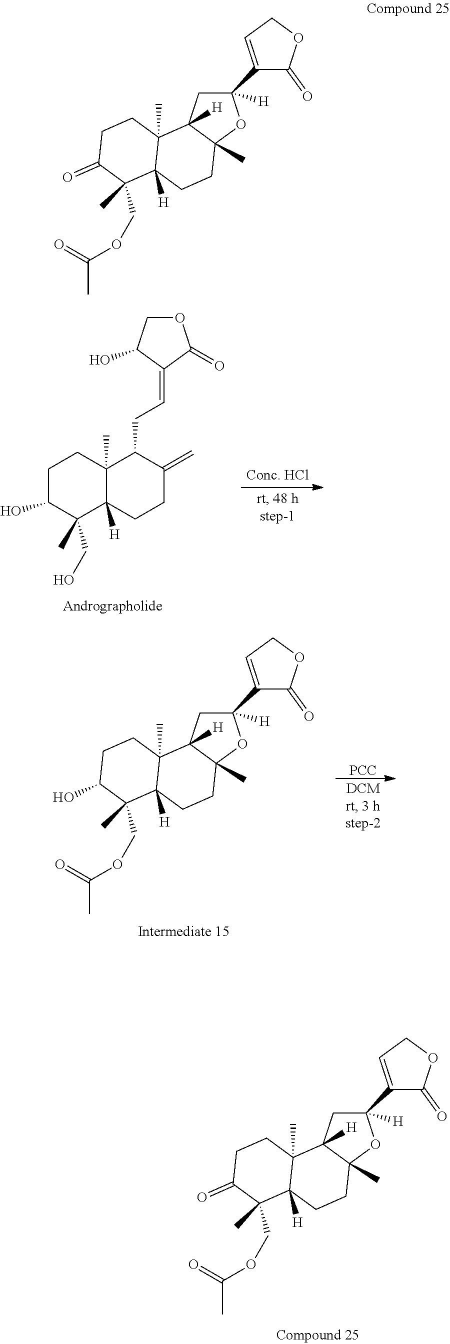

### Step 1: Synthesis of Intermediate 15

In a 500 mL single neck RBFlask ((3E,4S)-3-{2-[(1R,4aS,5R,6R,8aS)-6-hydroxy-5-(hydroxymethyl)-5,8a-dimethyl-2-methylidene-decahydronaphthalen-1-yl]ethylidene}-4-hydroxyoxolan-2-one) (Andrographolide) (50 g, 143 mmol, 1 eq.) and concentrated HCl (250 mL, 5 volumes) were mixed with stirring until the solution was completely dissolved. The resulting mixture was kept a side for 48 h. The reaction was monitored by TLC. After completion of the reaction, the reaction mixture was slowly poured into crushed ice, the precipitated or separated solids were stirred at rt for 2 h. Filtered the solids and washed with water (2000 mL), dried the solid under vacuum. The filtrate was concentrated under reduced pressure and the residue was purified by column chromatography over silica gel (100-200 mesh) by using a mixture of Acetone in DCM as eluent to afford ([(2S,3aR,5aS,6R,7R,9aR,9bS)-7-hydroxy-3a,6,9a-trimethyl-2-(2-oxo-2,5-dihydrofuran-3-yl)-dodecahydronaphtho[2,1-b]furan-6-yl]methyl acetate) (Intermediate 15). Yield: 1.9 g, 3.39%.

¹H NMR (400 MHz, CDCl₃): δ 7.28 (t, J=1.6 Hz, 1H), 4.81 (s, 2H), 4.71 (t, J=9.2 Hz, 1H), 4.39 (d, J=11.6 Hz, 1H), 4.18 (d, J=11.6 Hz, 1H), 3.30 (dd, J=10.8, 4.8 Hz, 1H), 2.46 (q, J=8.0 Hz, 1H), 2.21 (dd, J=13.2, 3.2 Hz, 1H), 2.06 (s, 3H), 2.01 (dd, J=8.0, 3.2 Hz, 2H), 1.79 (dd, J=13.2, 3.2 Hz, 1H), 1.76-1.67 (m, 2H), 1.61 (q, J=7.2 Hz, 2H), 1.55 (q, J=5.6 Hz, 2H), 1.17 (s, 3H), 1.12 (s, 3H), 1.03 (dd, J=16.8, 3.6 Hz, 2H), 0.98 (s, 3H). 13C NMR (100 MHz, CDCl₃): δ 172.5, 171.1, 143.2, 138.4, 82.6, 79.2, 73.2, 70.5, 65.1, 57.9, 52.9, 42.1, 39.3, 36.3, 35.8, 32.8, 31.4, 27.0, 22.7, 21.1, 18.5, 16.0.

### Step 2: Synthesis of Compound 25

To a solution of ([(2S,3aR,5aS,6R,7R,9aR,9bS)-7-hydroxy-3a,6,9a-trimethyl-2-(2-oxo-2,5-dihydrofuran-3-yl)-dodecahydronaphtho[2,1-b]furan-6-yl]methyl acetate) (Intermediate 15) (250 mg, 0.637 mmol, 1 eq.) in DCM (10 mL) was added PCC (275 mg, 1.27 mmol, 2 eq.) at rt. The resulting mixture was stirred at rt for further 3 h. The reaction was monitored by TLC. After completion of the reaction, the reaction mixture was concentrated under reduced pressure. The crude product obtained was purified by column chromatography by using (100-200 silica gel mesh) EtOAc in Hexanes as eluent to afford ([(2S,3aR,5aS,6R,9aR,9bS)-3a,6,9a-trimethyl-7-oxo-2-(2-oxo-2,5-dihydrofuran-3-yl)-dodecahydronaphtho[2,1-b]furan-6-yl]methyl acetate) (Compound 25), as white solid. Yield: 170 mg, (68.35%).

¹H NMR (400 MHz, CDCl₃): δ 7.31 (t, J=1.6 Hz, 1H), 4.84 (d, J=1.6 Hz, 2H), 4.78 (t, J=9.2 Hz, 1H), 4.62 (d, J=11.2 Hz, 1H), 4.99 (d, J=11.6 Hz, 1H), 2.79 (td, J=9.2, 6.0 Hz, 1H), 2.54 (q, J=8.0 Hz, 1H), 2.34 (dt, J=13.6, 1.6 Hz, 1H), 2.27 (dd, J=12.0, 6.2 Hz, 1H), 2.13-2.03 (m, 2H), 2.01 (s, 3H), 1.65-154 (m, 4H), 1.43-1.48 (m, 2H), 1.27 (s, 3H), 1.18 (s, 3H), 1.14 (s, 3H). 13C NMR (100 MHz, CDCl₃): δ 212.6, 172.4, 170.7, 143.2, 138.3, 82.5, 73.1, 70.5, 66.2, 57.0, 54.5, 51.4, 39.4, 36.0, 35.5, 34.9, 32.9, 31.4, 21.3, 20.7, 19.2, 15.6.

### Representative Synthesis of Compound 26

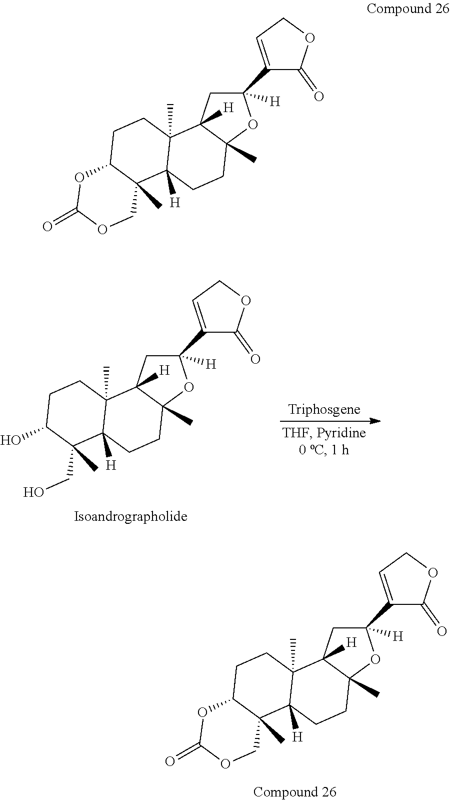

To a solution of 3-((2S,3aR,5aS,6R,7R,9aR,9bS)-7-hydroxy-6-(hydroxymethyl)-3a,6,9a-trimethyldodecahydronaphtho[2,1-b]furan-2-yl) furan-2(5H)-one (Isoandrographolide) (100 mg, 0.285 mmol, 1 eq.) in THF (5 mL) was added pyridine (0.069 mL, 0.856 mmol, 3 eq.) at rt. Cooled the reaction mixture to 0 °C, then added triphosgene (127 mg, 0.428 mmol, 1.5 eq.) lot wise. The resulting mixture was stirred at 0 °C for 1 h. Quenched the reaction mixture with saturated NaHCO₃ solution (20 mL), extracted with ethyl acetate (3×25 mL). The combined organic layer was dried over Na₂SO₄, filtered and concentrated under reduced pressure. The crude product obtained was purified by column chromatography by using (100-200 silica gel mesh) acetone in DCM as eluent to afford (4aR,4bS,6aR,8S,9aS,9bR,11aR)-4a,6a,9b-trimethyl-8-(2-oxo-2,5-dihydrofuran-3-yl) dodecahydro-4H-furo[3′,2′:5,6]naphtho[2,1-d][1,3]dioxin-2-one (Compound 26) as off-white solid. Yield: 60 mg (55.86%).

¹H NMR (400 MHz, CDCl₃, δ): 7.29 (d, J=6.4 Hz, 1H), 4.83-4.68 (m, 4H), 4.08 (t, 1H), 3.90 (d, J=11.2 Hz, 1H), 2.48-2.43 (q, 1H), 2.27-2.23 (q, 1H), 2.10-2.04 (m, 2H), 1.88-1.81 (q, 4H), 1.46-1.44 (m, 1H), 1.32 (s, 3H), 1.27 (d, 1H), 1.20-1.10 (m, 4H), 1.08-1.03 (m, 5H). ¹³C NMR (100 MHz, CDCl₃, δ): 172.4, 148.3, 143.3, 137.9, 73.0, 58.0, 50.5, 37.2, 35.5, 35.4, 34.7, 32.7, 31.6, 25.4, 23.9, 17.8, 16.3.

### Representative Synthesis of Compound 27

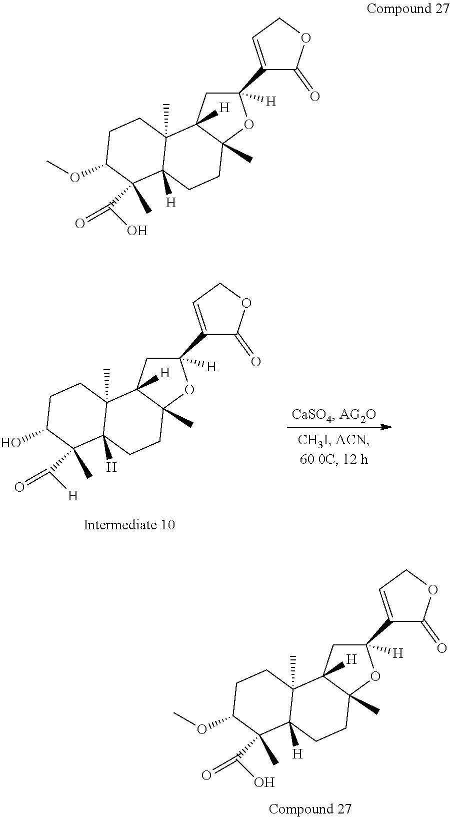

To a solution of (2S,3aR,5aS,6S,7R,9aR,9bS)-7-hydroxy-3a,6,9a-trimethyl-2-(2-oxo-2,5-dihydrofuran-3-yl) dodecahydronaphtho[2,1-b]furan-6-carbaldehyde (Intermediate 10) (1 g, 2.87 mmol, 1 eq.) in ACN (20 mL) were added Ag₂O (3.35 g, 14.3 mmol, 5 eq.), CaSO₄ (2.34 g, 17.2 mmol, 6 eq.) and CH₃I (8.15 g, 57.4 mmol, 20 eq.) at rt. The resulting mixture was stirred at rt for 30 h, then heated at 60 °C for further 12 h. The reaction mixture was monitored by TLC. After completion of the reaction, filtered the reaction mixture through a pad of silica gel and washed with ethyl acetate (100 mL). The combined filtrate was concentrated under reduced pressure. The crude product obtained was purified by column chromatography by using (100-200 silica gel mesh) EtOAc in Hexanes as eluent to afford (2S,3aR,5aS,6S,7R,9aR,9bS)-7-methoxy-3a,6,9a-trimethyl-2-(2-oxo-2,5-dihydrofuran-3-yl) dodecahydronaphtho[2,1-b]furan-6-carboxylic acid (Compound 27), as off-white solid. Yield: 350 mg (32.22%).

¹H NMR (400 MHz, CDCl₃, δ): 7.28 (s, 1H), 4.81 (s, 2H), 4.72 (brs, 1H), 3.67 (s, 3H), 3.39 (d, J=12 Hz, 1H), 3.11 (t, 1H), 2.50-2.45 (q, 1H), 2.22 (d, J=14.8 Hz, 1H), 2.09-1.99 (m, 2H), 1.79 (s, 5H), 1.54 (d, J=7.2 Hz, 1H), 1.43 (s, 3H), 1.39 (d, 1H), 1.10 (s, 4H), 1.04 (d, 1H), 0.84 (s, 3H). ¹³C NMR (100 MHz, CDCl₃, δ): 178.2, 172.5, 143.1, 138.5, 82.5, 78.4, 73.3, 70.5, 56.8, 53.8, 51.3, 49.0, 39.9, 36.6, 36.1, 32.9, 31.2, 27.9, 23.9, 19.7, 14.2.

### Representative Synthesis of Compound 28

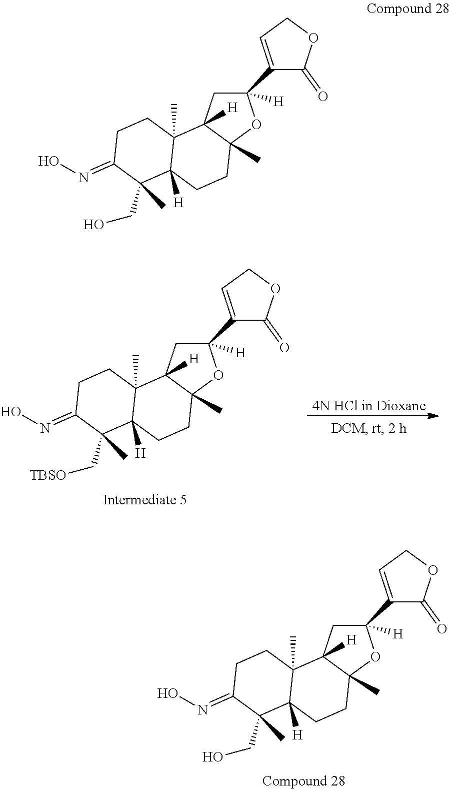

To a solution of 3-((2S,3aR,5aS,6S,9aR,9bS,E)-6-(((tert-butyldimethylsilyl)oxy)methyl)-7-(hydroxyimino)-3a,6,9a-trimethyldodecahydronaphtho[2,1-b]furan-2-yl) furan-2(5H)-one (Intermediate 5) (1 g, 2.09 mmol, 1 eq.) in DCM (10 mL) was added 4N HCl in Dioxane solution (2.61 mL, 5 eq.) at 0 °C. The resulting reaction mixture was stirred at rt for 2 h. The reaction was monitored by TLC. After completion of the reaction, the reaction mixture was diluted with DCM (50 mL), basified (up to PH=8) with saturated NaHCO₃ solution (50 mL), then extracted with DCM (3×40 mL). The combined organic layer was dried over Na₂SO₄, filtered and concentrated under reduced pressure to afford 3-((2S,3aR,5aS,6S,9aR,9bS,E)-7-(hydroxyimino)-6-(hydroxymethyl)-3a,6,9a-trimethyldodecahydronaphtho[2,1-b]furan-2-yl) furan-2(5H)-one (Compound 28), as white colour solid. Yield: 650 mg (85.43%).

¹H NMR (400 MHz, CDCl₃, δ): 7.85 (brs, 1H), 7.30 (d, J=2.0 Hz, 1H), 4.83 (d, J=1.6 Hz, 2H), 4.75 (t, 1H), 3.84 (d, J=10.8 Hz, 1H), 3.48 (d, J=8.4 Hz, 1H), 3.05-2.98 (dd, 1H), 2.76 (brs, 1H), 2.54-2.49 (q, 1H), 2.32-2.23 (m, 2H), 2.13-2.07 (m, 1H), 1.57-1.53 (m, 1H), 1.36-1.32 (t, 1H), 1.27 (s, 3H), 1.22-1.15 (m, 1H), 1.13 (s, 3H), 1.05 (s, 3H). ¹³C NMR (100 MHz, CDCl₃, δ): 172.5, 164.7, 143.1, 138.5, 82.5, 73.1, 70.5, 65.4, 56.7, 53.0, 44.6, 36.9, 36.2, 35.4, 32.9, 31.4, 23.6, 18.5, 18.0, 15.5.

### Representative Synthesis of Compound 29

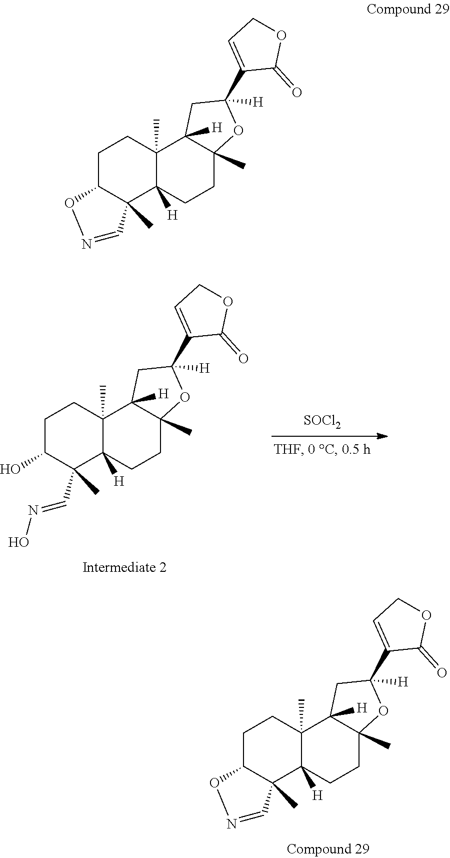

To solution of (E)-7-hydroxy-3a,6,9a-trimethyl-2-(2-oxo-2,5-dihydrofuran-3-yl) dodecahydronaphtho[2,1-b]furan-6-carbaldehyde oxime (Intermediate 2) (800 mg, 2.2 mmol, 1.0 eq.) in THF (10 ml), were added SOCl₂ (239 μL, 3.3 mmol, 1.5 eq.) drop wise at 0 °C. Then resulting reaction mixture was stirred at 0 °C for 30 min. The reaction was monitored by TLC. After completion of the reaction, saturated NaHCO₃ solution was added to reaction mixture and extracted with ethyl acetate (2×20 mL), the combined organic layer was dried over Na₂SO₄, filter and concentration under reduced pressure. The obtained crude was purified through column chromatography by using silica (100-120 mesh), ethyl acetate in hexane as elute to afford 3-((3aR,3bS,5aR,7S,8aS,8bR,10aR)-3a,5a,8b-trimethyl-3a,3b,4,5,5a,7,8,8a,8b,9,10,10a-dodecahydrofuro[3′,2′:5,6]naphtho[1,2-d]isoxazol-7-yl) furan-2(5H)-one (Compound 29), as off-white solid. Yield: 70 mg (9.21%).

¹H NMR (400 MHz, CDCl₃, δ): 7.26 (d, J=5.2 Hz, 1H), 7.12 (s, 1H), 4.80 (s, 2H), 4.71 (t, 1H), 4.15-4.11 (q, 1H), 2.49-2.43 (q, 1H), 2.27 (d, J=10.8 Hz, 1H), 2.08-2.00 (m, 1H), 1.84 (m, 1H), 1.61-1.42 (m, 7H), 1.20 (m, 1H), 1.15 (s, 3H), 1.11 (s, 3H), 0.93 (s, 3H), 0.87 (d, 1H). ¹³C NMR (100 MHz, CDCl₃, δ): 172.4, 153.6, 143.2, 138.4, 84.2. 82.6, 73.1, 70.5, 56.0, 53.6, 48.7, 35.8, 35.1, 34.8, 32.5, 31.5, 25.1, 24.2, 19.9, 14.2.

### Representative Synthesis of Compound 30

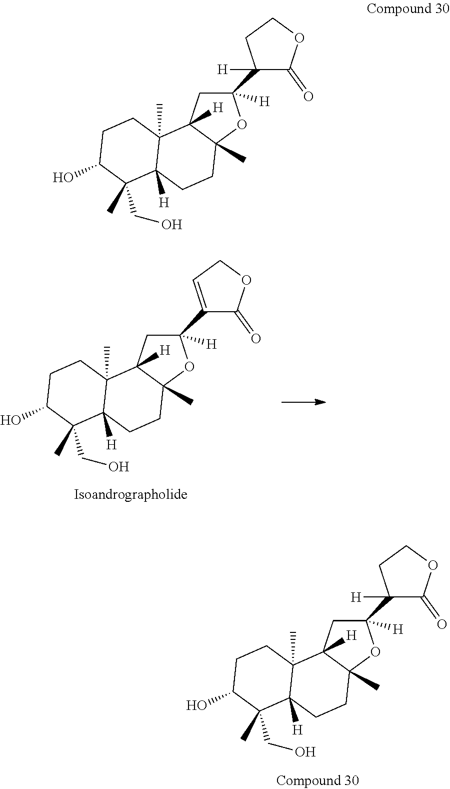

To a solution of 3-{(1R,2S,4S,6R,9S,10R,11R)-11-hydroxy-10-(hydroxymethyl)-1,6,10-trimethyl-5-oxatricyclo[7.4.0.0^{2,6}]tridec-4-yl}-2(5H)-furanone (Isoandrographolide) (2 g, 5.71 mmol, 1 eq.) in Methanol (80 mL) at rt was added Pd/C (1 g). The resulting mixture was stirred at rt under hydrogen gas pressure at rt for 12 h. The reaction mixture was monitored by TLC. After completion of the reaction, reaction mixture was filtered through celite bed and washed the bed with MeOH (100 mL). The combined filtrate was concentrated under reduced pressure. The crude product obtained was purified by column chromatography by using (100-200 silica gel mesh) MeOH in DCM as eluent to afford the desired product 3-{(1R,2S,6R,9S,10R,11R)-11-hydroxy-10-(hydroxymethyl)-1,6,10-trimethyl-5-oxatricyclo[7.4.0.0^{2,6}]tridec-4-yl}-4,5-dihydro-2(3H)-furanone (Compound 30). Yield: 850 mg (42.26%).

¹H NMR (400 MHz, CDCl₃, δ): 5.04 (d, J=6.0 Hz, 1H), 4.3-4.2 (m, 1H), 4.18-4.10 (m, 2H), 3.95-3.71 (m, 2H), 3.21-3.19 (m, 1H), 2.78-2.71 (m, 1H), 2.32-2.24 (m, 1H), 2.14-1.82 (m, 4H), 1.62-1.41 (m, 6H), 1.10 (s, 3H), 1.06 (s, 3H), 1.02-0.91 (m, 2H), 0.83 (s, 3H).

### Representative Synthesis of Compound 31

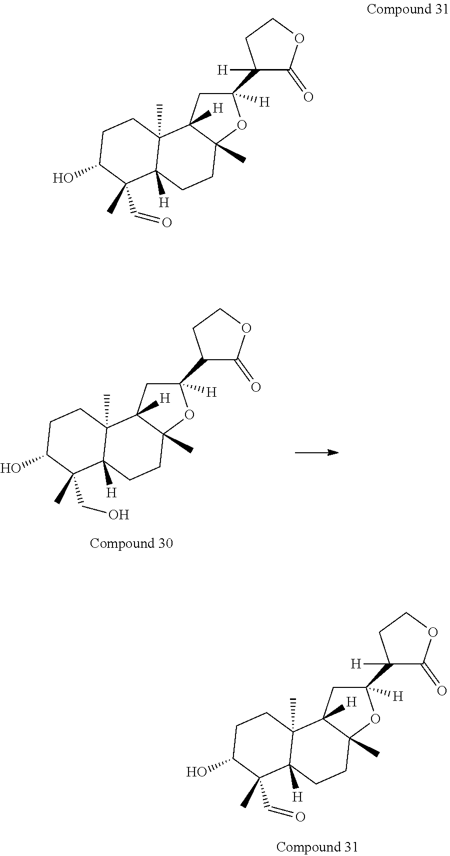

To a solution of 3-{(1R,2S,6R,9S,10R,11R)-11-hydroxy-10-(hydroxymethyl)-1,6,10-trimethyl-5-oxatricyclo[7.4.0.0^{2,6}]tridec-4-yl}-4,5-dihydro-2(3H)-furanone (Compound 30) (0.4 g, 1.13 mmol, 1 eq.) in DCM (10 mL) were added TEMPO (35.5 mg, 0.2227 mmol, 0.2 eq.), TBAI (83.8 mg, 0.227 mmol, 0.2 eq.), NCS (227 mg, 1.7 mmol, 1.5 eq.), 0.5 M Aq. NaHCO₃ (5 mL) and 0.05 M Aq. K₂CO₃ (5 mL) solution at 0 °C. The resulting mixture was stirred at rt for 2 h. The reaction mixture was monitored by TLC. After completion of the reaction, the reaction mixture was quenched with water (50 ml) and extracted with DCM (3×30 mL), organic layer was washed with saturated Sodium thiosulfate solution (3×40 mL). The combined organic layer was dried over Na₂SO₄, filtered and concentrated under reduced pressure. The crude product obtained was purified by column chromatography by using (100-200 silica gel mesh) EtOAc in Hexanes as eluent to afford (2S,3aR,5aS,6S,7R,9aR,9bS)-7-hydroxy-3a,6,9a-trimethyl-2-(2-oxooxolan-3-yl)-dodecahydronaphtho[2,1-b]furan-6-carbaldehyde (Compound 31), as a yellow solid. Yield: 300 mg (75.43%).

¹H NMR (400 MHz, CDCl₃, δ): 9.92 (s, 1H), 5.03=(d, J=4.0 Hz, 1H), 4.27 (dt, J1=2.4 Hz, J2=8.4 Hz, 1H), 4.13 (dd, J1=6.8 Hz, J2=9.2 Hz, 1H), 3.76 (brs, 1H), 3.32-3.25 (m, 1H), 2.62-2.53 (m, 1H), 2-41-2.32 (m, 1H), 1.98-1.82 (m, 2H), 1.76-1.52 (m, 4H), 1.48-1.44 (m, 2H), 1.47-1.40 (m, 4H), 1.10-1.01 (m, 6H), 0.45 (s, 3H), 0.23-0.26 (m, 1H).

### Example 2

### Reduction of Fibrosis in Pulmonary Fibrosis Model

At Day 0, prophylactic treatment of mice with Compound 3 or pirfenidone by PO was performed. From Day 0 to Day 20, the prophylactically treated mice were intratracheally instilled with bleomycin. At Day 21, the mice were euthanized.

As depicted in FIGS. 1A-1F, a series of bar graphs illustrate the reduction in fibrotic symptoms in a bleomycin-induced pulmonary fibrosis model upon treatment with Compound 3 at 1 mpk, 3 mpk, 10 mpk, and 100 mpk as compared to a normal control, treatment-free control (bleomycin), and pirfenidone (comparative) at 100 mpk. Parameters such as collagen deposition, collagen accumulation, collagen fiber formation, inflammatory cytokine expression, and inflammatory cell infiltration were measured. The results indicated a notable decrease in these parameters, suggesting the compound's potential in treating fibrotic diseases.

### Example 3

### Therapeutic Effects in Bleomycin-Induced Pulmonary Fibrosis Model

Prophylactic group (P): At Day 0, prophylactic treatment of mice (P) with Compound 1 or pirfenidone by PO was performed. From Day 0 to Day 20, the prophylactically treated mice (P) were intratracheally instilled with bleomycin. At Day 21, the mice (P) were euthanized.

Treatment group (T): Likewise, from Day 0 to Day 7, naïve mice (T) were intratracheally instilled with bleomycin. At Day 7, the naïve mice were treated with Compound 1. Bleomycin instillation continued to Day 20. At Day 21, the mice (T) were euthanized.

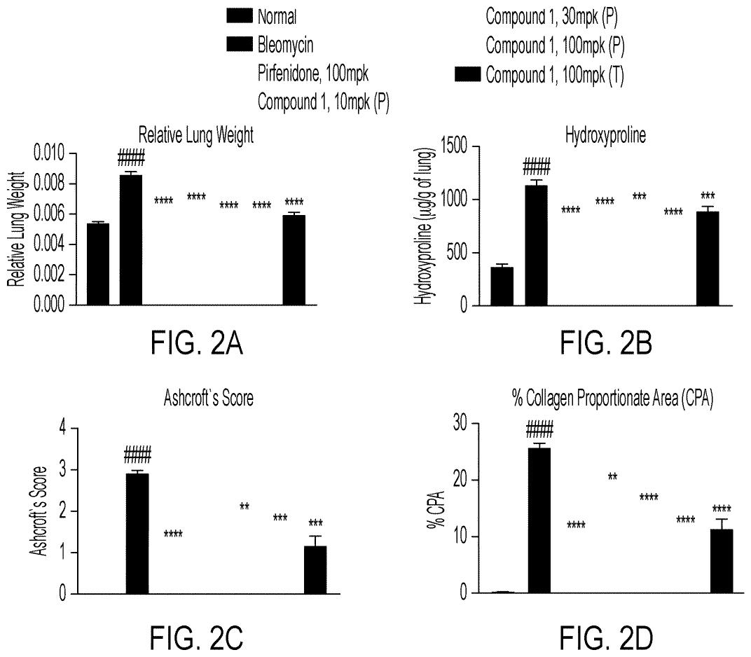

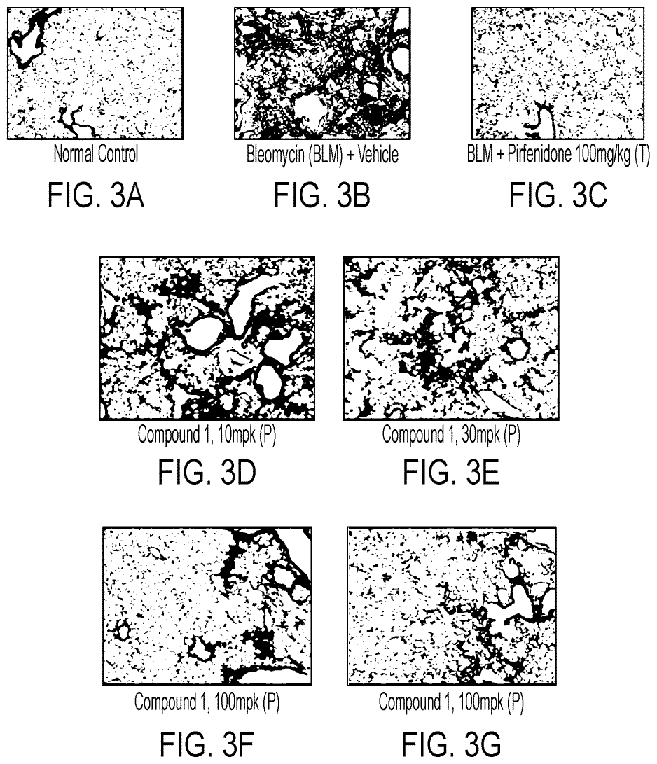

FIGS. 2A-2D and FIGS. 3A-3G illustrate the therapeutic effects of Compound 1 at 10 mpk (P), 30 mpk (P), 100 mpk (P), and 100 mpk (T), as compared to a normal control, treatment-free control (bleomycin), and pirfenidone (comparative) at 100 mpk (P) in a bleomycin-induced model of pulmonary fibrosis. The bar graphs and histological images demonstrate the ability of Compound 1 to reduce hydroxyproline content, lower the Ashcroft score, and decrease inflammatory cell infiltration in lung tissue. The histological images with H&E staining at 100× magnification provide a visual confirmation of the reduced fibrosis and improved lung tissue morphology in the compound-treated group compared to the control.

### Example 4

### Inhibition of Inflammatory Cytokines

At time point −1 hour, prophylactic treatment of mice by PO with Compound 3 or dexamethasone was performed. From time point −0.5 hours to 0 hours, neutrophil activation was induced with aerosolized LPS at 1 mg/mL. At timepoint 4 hours, blood, BALF, and lung tissue were harvested.

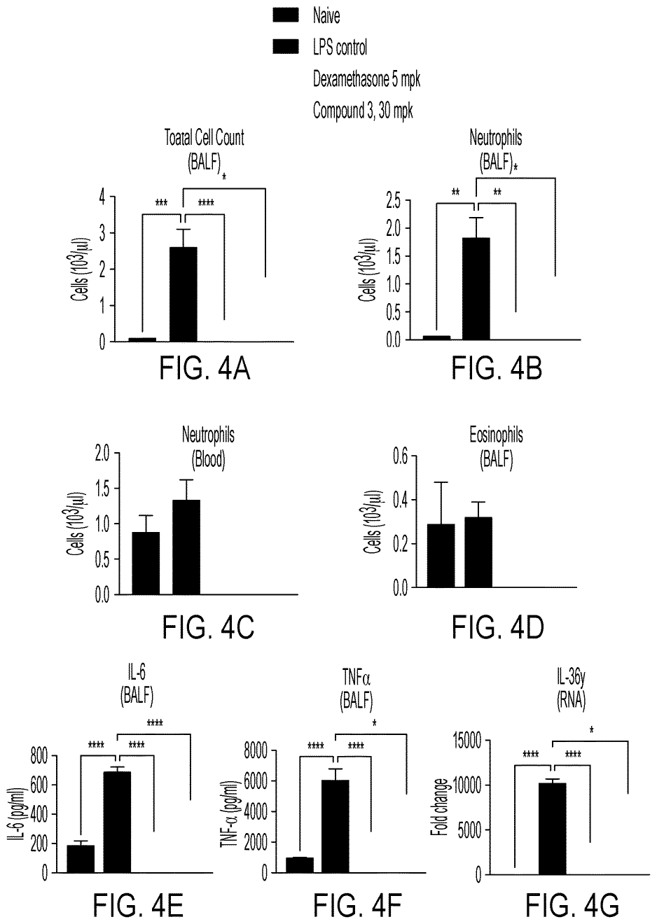

FIGS. 4A-4G present the effects of Compound 3 at 30 mpk as compared to naive control, treatment-free control (LPS), and dexamethasone (comparative) at 5 mpk on inflammatory cytokines in a lipopolysaccharide (LPS)-induced acute lung injury model. The bar graphs compare the levels of TNF-α, IL-6, IL-36γ, Total cell count (BALF), neutrophils (BALF), neutrophils (blood), and eosinophils (BALF), MIP-2. Compound 3 demonstrated a dose-dependent reduction in cytokine levels, indicating their anti-inflammatory properties.

### Example 5

### Impact on Inflammation and Cell Counts

At Day 0, prophylactic treatment of mice with Compound 3 or pirfenidone by PO was performed. From Day 0 to Day 9, the prophylactically treated mice were intratracheally instilled with bleomycin. At Day 10, the mice were euthanized.

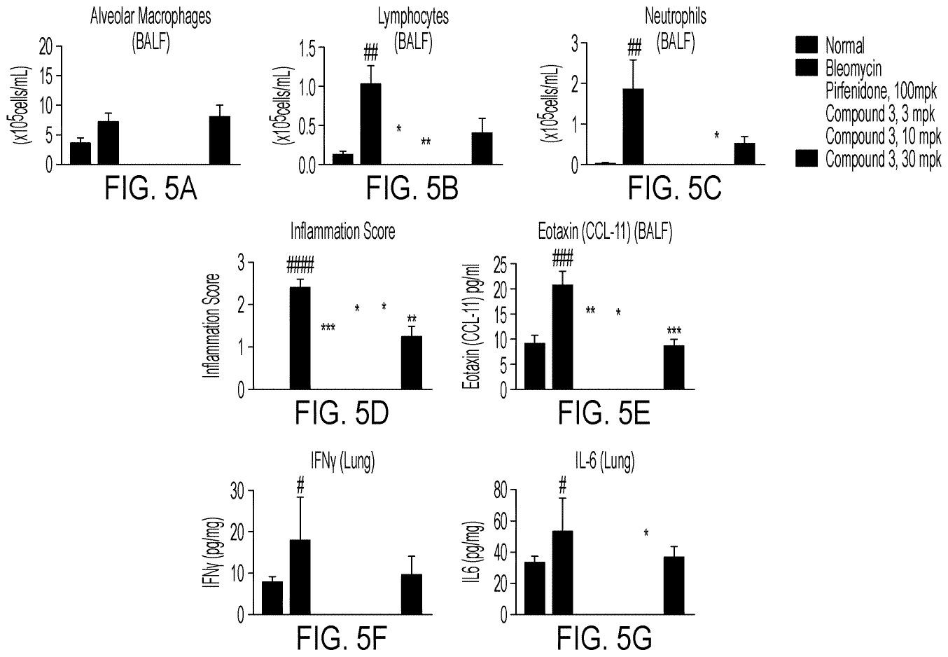

As shown in FIGS. 5A-5G, the impact of Compound 3 at 3 mpk, 10 mpk, and 30 mpk as compared to normal control, treatment-free control (bleomycin), and pirfenidone (comparative) at 100 mpk on various cell counts associated with inflammation was evaluated. The bar graphs compare alveolar macrophage counts, neutrophil counts, lymphocyte counts, inflammation score, eotaxin (BALF), INFγ (lung), and IL-6 (lung). The exemplary compound showed a reduction in inflammation and cell counts, suggesting its potential as an anti-inflammatory agent.

### Example 6

### Dose-Dependent Impact on Biomarkers

Cryopreserved human precision-cut lung slices (hPCLS) were prepared and used to analyze biomarkers in inflammatory and fibrotic models. Information of the donors that provided the hPCLS is provided below.

TABLE 2
hPCLS Donor Information

| | | |
|---|---|---|
| Lung Health | Normal | IPF-ILD |
| Age | 59 | 65 |
| Gender | M | F |
| Ethnicity | hispanic | other |
| Height (cm) | 163 | NA |
| Weight (kg) | 88.6 | NA |
| PCLS Thickness | 437 ± 50 | 475 ± 84 |
| PCLS Diameter | −8 mm | −8 mm |

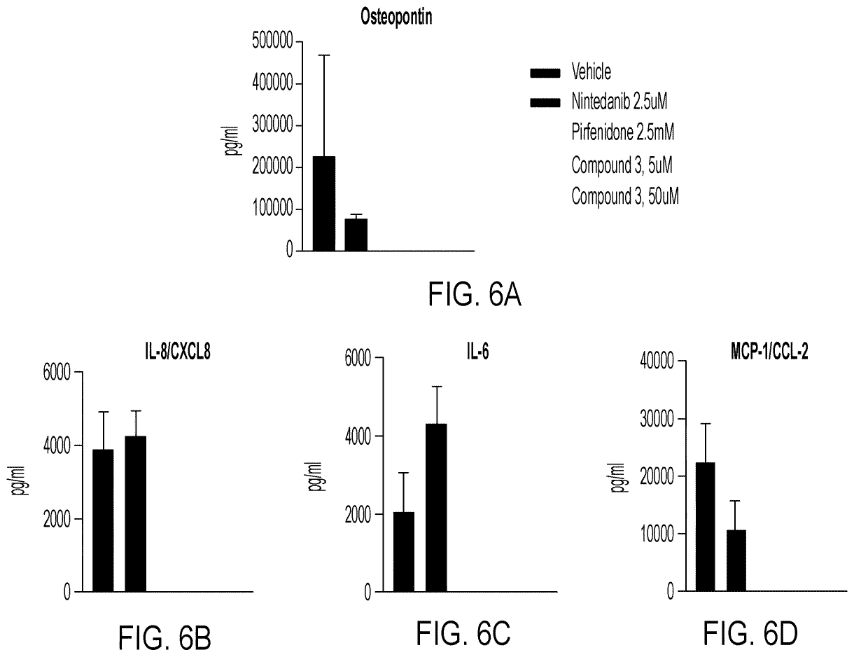

FIGS. 6A-6D illustrate the dose-dependent impact of Compound 3 at 5 μM and 50 uM as compared to vehicle control, nintedanib at 2.5 μM (comparative), and pirfenidone at 2.5 uM (comparative) on biomarkers associated with inflammation and fibrosis in human pulmonary cells. The data indicate that increasing concentrations of the compounds result in a corresponding decrease in the levels of biomarkers, such as IL-8/CXCL8, IL-6, and MCP-1/CCL-2, which are implicated in the pathogenesis of fibrotic diseases.

### Example 7

### Inhibition of Neutrophil Chemotaxis

Freshly isolated healthy human neutrophils were isolated and utilized. The neutrophils were treated with IL-8 at 2 nM or fMLP at 100 nM to induced chemotaxis.

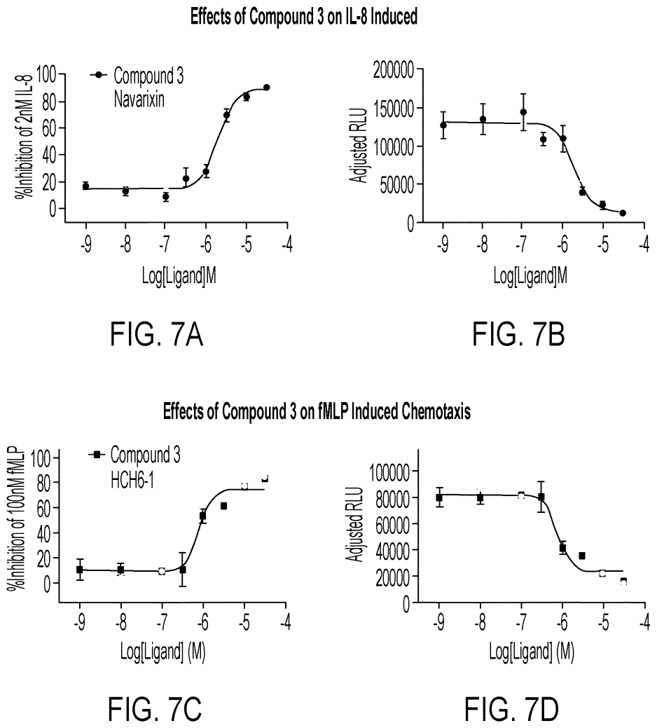

As shown in FIGS. 7A-7D, Compound 3 exhibits concentration-dependent inhibition of neutrophil chemotaxis induced by various stimuli, including IL-8, and fMLP. FIGS. 7A and 7B display the percentage of inhibition of 2 nm of IL-8 induced chemotaxis at different compound concentrations of Compound 3 as compared to Navarixin (comparative). FIGS. 7C and 7D display the percentage of inhibition of 100 nm fMLP induced chemotaxis at different compound concentrations of Compound 3 as compared to HCH6-1 (comparative). Table 3 below provides the IC50 values for each stimulus, providing a comparative measure of the compounds' efficacy.

TABLE 3
IC50 Values

| | | IC₅₀ | |
|---|---|---|---|
| Navarixin | Donor 1 | 1.23 | μM |
| (IL-8) | Donor 2 | 2.69 | μM |
| | Donor 3 | 3.16 | μM |
| | All | 2.36 | μM |
| Compound 3 | Donor 1 | 1.41 | μM |
| (IL-8) | Donor 2 | 3.16 | μM |
| | Donor 3 | 1.62 | μM |
| | All | 2.06 | μM |
| HCH6-1 | Donor 1 | 452 | nM |
| (fM LP) | | | |
| Compound 3 | Donor 1 | 798 | nM |
| (fM LP) | | | |

### Example 8

### Efficacy in DSS Mouse Model of Colitis

From Day-3 to Day 8, mice were treated with Compound 1, fingolimod, or mesalamine. From Day-3 to Day 0, mice were randomized and acclimated to their environment while assessing a disease activity index (DAI). At Day 1, 2% w/w of DSS in water was administered to the mice by PO. From Day 1 to Day 7, DAI continued to be assessed. At Day 8, the mice were euthanized post the final dose of Compound 1, fingolimod, or mesalamine.

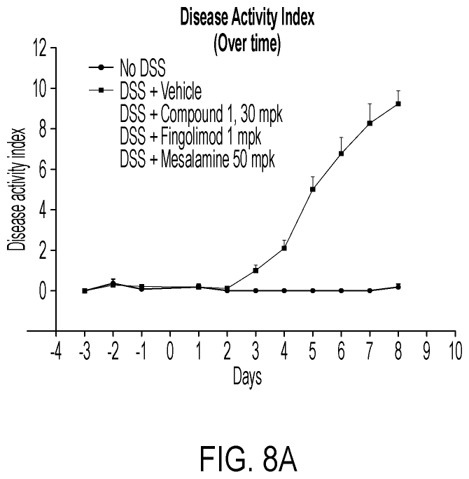

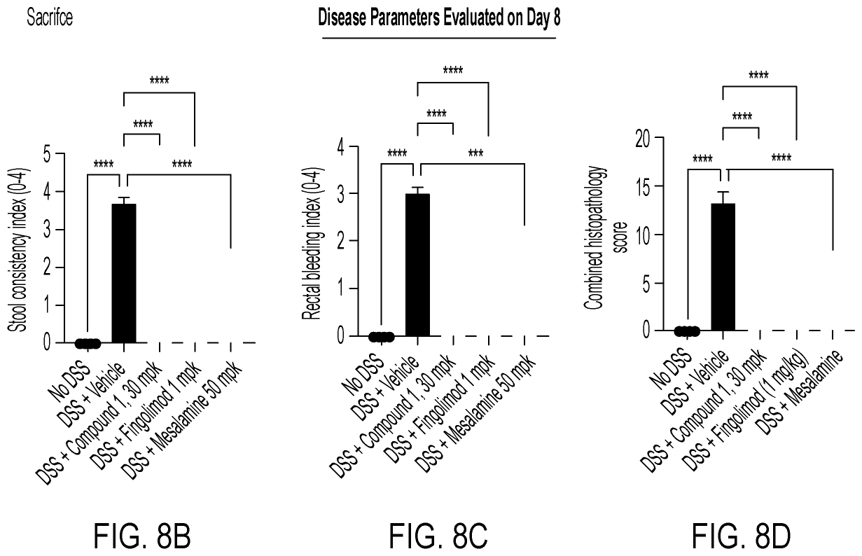

FIGS. 8A-8D present the efficacy of Compound 1 at 30 mpk as compared to vehicle control, DSS control, fingolimod at 1 mpk (comparative), and mesalamine at 50 mpk (comparative) in a DSS mouse model of colitis. The graphs show the effect of Compound 1 on disease activity index, stool consistency, rectal bleeding index, and combined histopathology. The dose-response curve over time indicates that the compounds effectively mitigate the symptoms of colitis, such as weight loss and increased disease severity, at various dosages.

### Example 9

### Reduction of Fibrosis in Peritoneal Fibrosis Model

From Day 1 to Day 21, 0.1% w/w of chlorhexidine gluconate (CG) in water was administered, every other day, to the mice by PO to induce peritoneal fibrosis. From Day 1 to Day 21, mice were treated with Compound 1 or an inhibitor of TGFβ receptor I (ALK5) (SB525334). At Day 22, the mice were euthanized.

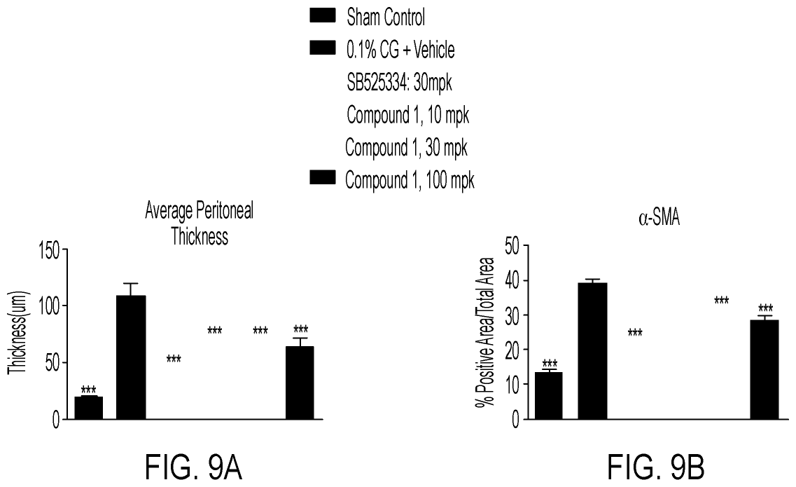

FIGS. 9A and 9B illustrate the effect of Compound 1 at 10, mpk, 30, mpk, and 100 mpk as compared to sham control, 0.1% chlorhexidine gluconate (CG) and vehicle, and an inhibitor of TGFβ receptor I (ALK5) (SB525334) at 30 mpk (comparative), and mesalamine at 50 mpk (comparative) on average peritoneal thickness and α-SMA expression in a mouse model of peritoneal fibrosis. The data demonstrate that Compound 1 lead to a reduction in these fibrosis markers, suggesting their potential to treat fibrotic conditions beyond pulmonary fibrosis, such as peritoneal fibrosis.

### Example 10

### Efficacy in Psoriasis Mouse Model

From Day 0 to Day 7, 5% w/w of imiquimod (IMQ) in water was administered to the mice by PO to induce psoriasis. From Day 0 to Day 7, mice were treated with Compound 1, clobetasol, or dexamethasone. At Day 7, the mice were euthanized.

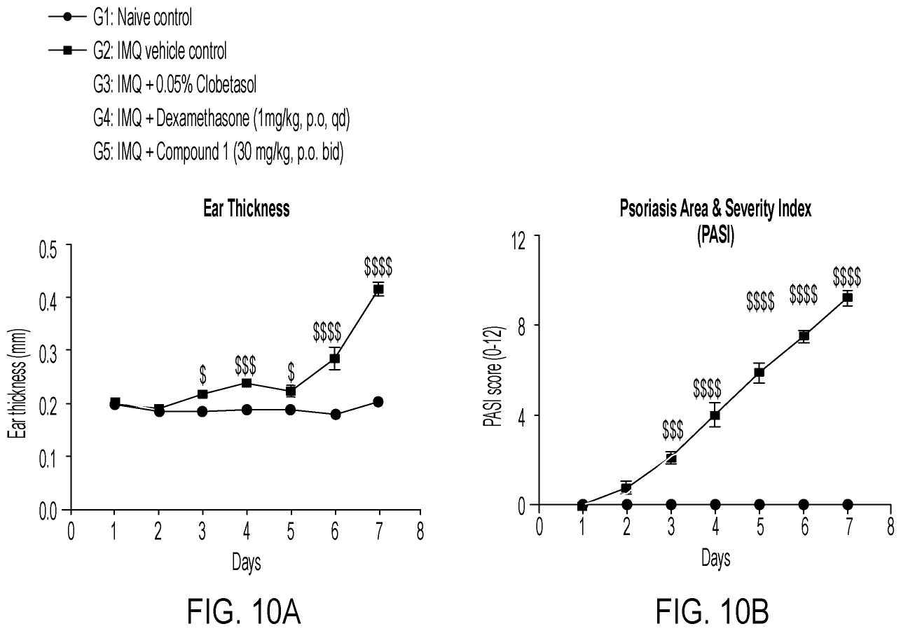

FIGS. 10A and 10B illustrate the effect of Compound 1 at 30 mpk (P.O., BID) as compared to naive control, vehicle control, 0.05% clobetasol, and dexamethasone at 1 mpk (P.O., QD) on psoriasis in a mouse model. The graphs show the changes in ear thickness and Psoriasis Area and Severity Index (PASI) score over time. The data demonstrate that Compound 1 treated mice exhibited improvements in skin condition and a reduction in symptoms, highlighting the potential of Compound 1 in treating dermatological conditions.

### Example 11

### Effects on Atopic Dermatitis

From Day 1 to Day 14, 2 nmol in 100% ethanol, 50 μl/mouse/back skin (MC903) was administered every other day to mice to induce atopic dermatitis. From Day 1 to Day 14, mice were treated with Compound 1 or clobetasol. At Day 14, the mice were euthanized.

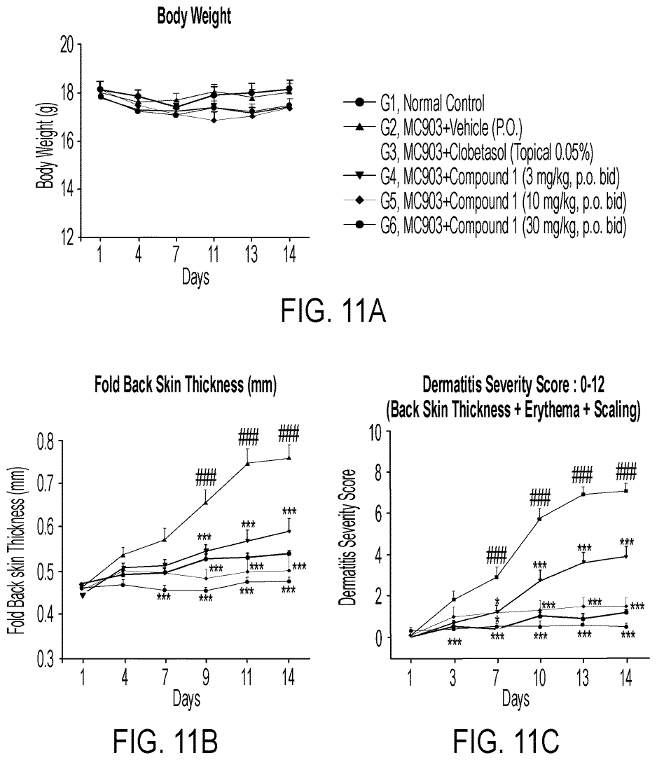

FIGS. 11A-11C illustrate the effects of Compound 1 at 3 mpk, 10 mpk, and 30 mpk (PO, BID) as compared to normal control, vehicle control, and 0.05% clobetasol (topical, QD) (comparative) on atopic dermatitis in a mouse model. The graphs demonstrate the influence of Compound 1 on body weight, fold back skin thickness and dermatitis severity score. The Compound 1-treated groups showed a decrease in dermatitis severity and skin thickness, indicating the effectiveness of Compound 1 in ameliorating symptoms of atopic dermatitis.

### Example 12

Compound 2 improves MC-903-induced Atopic Dermatitis in Mice comparable to high-potency topical corticosteroids

Experimental details: Preclinical animal model of atopic dermatitis was developed by MC-903, a vitamin-D analogue. In the current study, the test item Compound 2 was screened to evaluate its efficacy in MC-903 induced AD model. 2 nmol of MC-903 was topically applied to 8-10 weeks old female BALB/c mouse from G2-G7 group at a volume of 50 μl/mouse on back skin on every alternate day for 14 days to induced disease. For the G1 group, (Normal+ethanol control) the same volume of ethanol that was used to solubilize MC-903 was applied. Clobetasol (0.05%, 20 mg) was applied topically once a day to G3 group animals. Compound 2 was administered twice a day to respective treatment groups (G4-G7) for 14 days. On the day of MC-903 administration, test compounds were dosed one hour prior to MC-903 insult. Clinical scores to assess the disease intensity were measured from visual examination for erythema, scaling and back skin thickness using dial caliper. All the scores were carried by blinded observers to treatment groups. Cumulative dermatitis severity scores were calculated by adding up the individual scores of erythema, scaling & thickness. On day-14 of the study, all animals were terminally bled under mild anesthesia followed by humane sacrifice and back skin tissue was dissected. Required skin tissue samples were collected in fixative and part of the tissue was flash-frozen in liquid nitrogen and stored at −80 °C. Collected tissue samples were processed and used for biomarker analysis using gene and protein expression analysis by qPCR & ELISA, pharmacokinetic analysis by LC-MS and histopathology studies.

Results & Discussion: MC-903 induces the activation and proliferation of epidermal keratinocytes and promotes the expression of thymic stromal lymphopoietin (TSLP) expression, a master switch for Th2 allergic inflammation, including AD. This preclinical mouse model utilizes the topical application of MC903 on the skin, which induces skin and systemic AD-like inflammation. The phenotypes closely mimic most of the clinical, skin barrier-related, histological, and immunological characteristics observed in AD patients. Other AD characteristics, such as skin eczematous-like lesions with xerosis and pruritus, increased serum IgE levels, inflammatory infiltrates in the skin and draining lymph nodes, were also observed in mice treated with MC-903. Thus, the MC903-induced AD mouse model is a promising preclinical research tool for studying immunologic abnormalities involved in AD development or maintenance or preclinical studies for unravelling new therapeutic strategies for this disease.

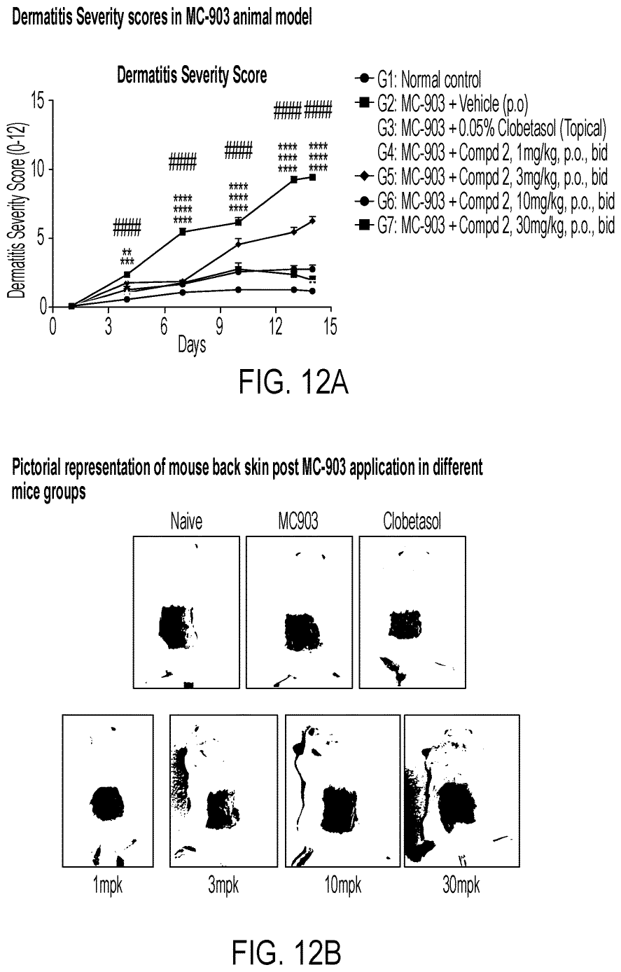

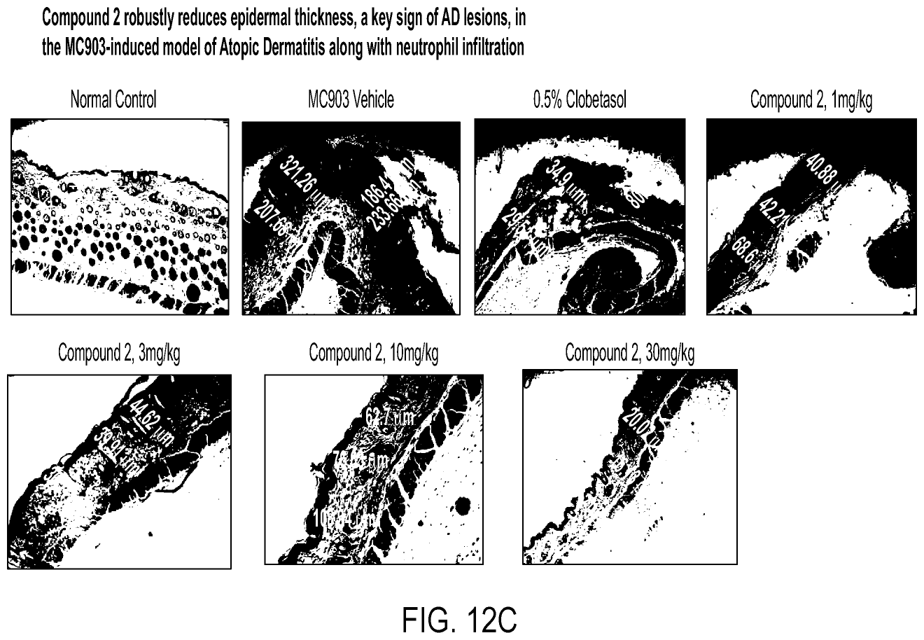

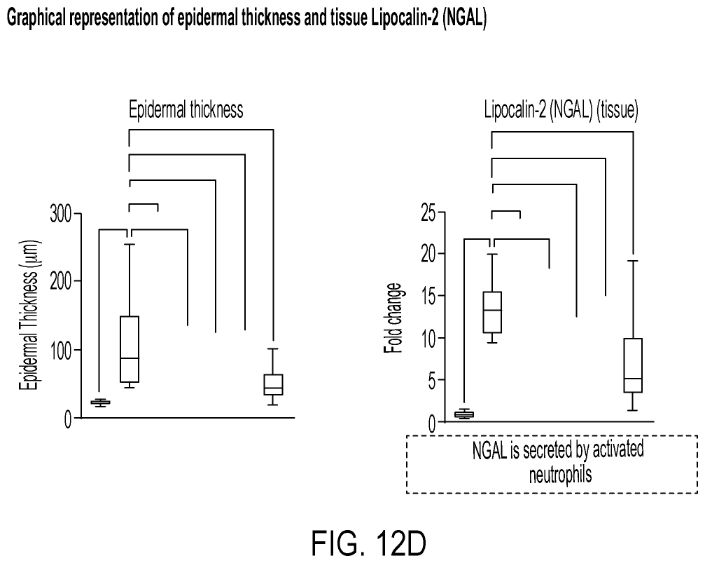

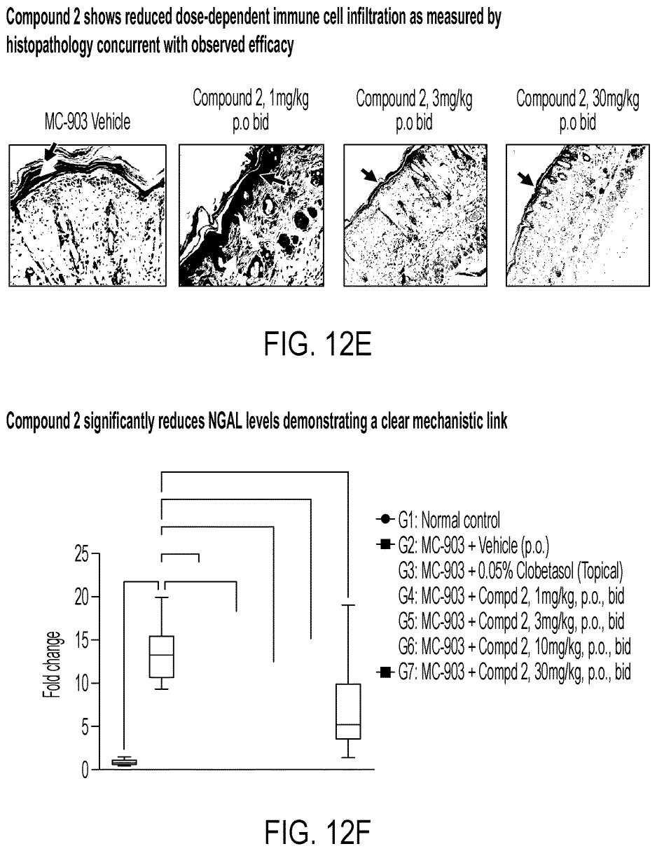

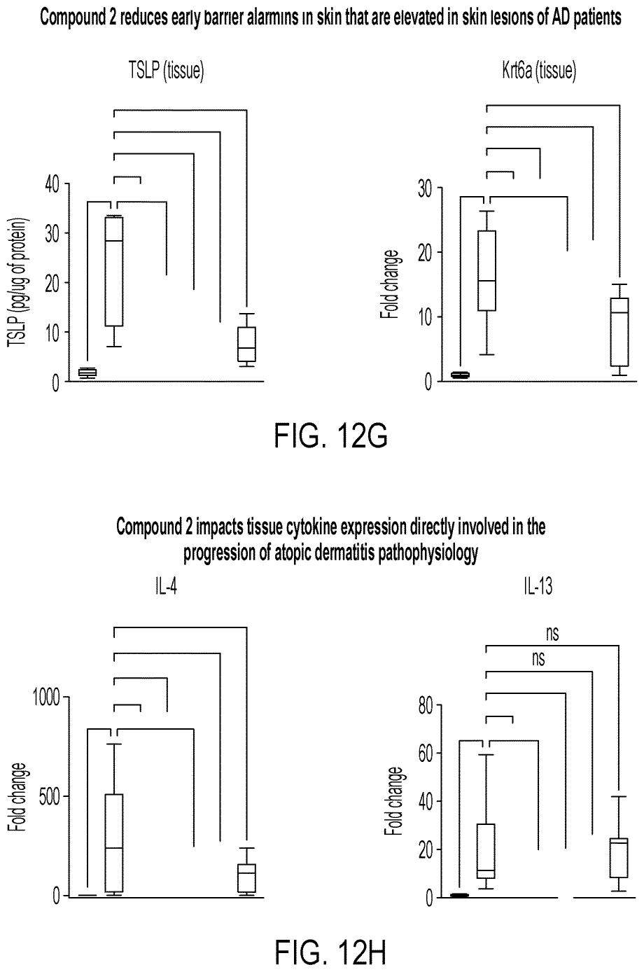

In the current study, MC-903 topical application on mouse back skin significantly increased back fold skin thickness and dermatitis severity scores during in-life phase of the study in all animals except in normal control group from D-1 to D-14. Compound 2 showed dose dependent decrease in dermatitis severity scores when compared with MC-903 vehicle control (FIG. 12A). Representative images from all the groups were presented in FIG. 12B.

MC-903 topical insult significantly increased the expression of all Th2 cytokines and epidermal barrier proteins such as Tissue TSLP, IL-4, IL-13, Krt6a (FIG. 12G & FIG. 12H), neutrophil markers such as Lipocalin-2 and NGAL (FIG. 12D & FIG. 12F) and itch markers such as IL-31 (FIG. 12I). MC-903 also significantly increased the epidermal thickness (FIG. 12D) and histopathological changes in keratin, epidermis and dermal skin layers such as immune cell infiltration, hyperkeratosis, parakeratosis, lengthening and clubbing of rete ridges and final histopathology scores (an increase in score means increase in dermatitis disease intensity) in back skin, when compared with normal control (FIG. 12C).

Compound 2 as a prophylactic treatment significantly reduced the expression of all Th2 cytokines and epidermal barrier proteins such as Tissue TSLP, IL-4, IL-13, Krt6a (FIG. 12G & FIG. 12H), neutrophil markers such as Lipocalin-2 and NGAL (FIG. 12D & FIG. 12F) and Itch markers such as IL-31 (FIG. 12I). Compound 2 treatment also significantly reduced the epidermal thickness (FIG. 12D) and histopathological changes in keratin, epidermis and dermal skin layers such as immune cell infiltration, hyperkeratosis, parakeratosis, lengthening and clubbing of rete ridges and final histopathology scores (an increase in score means increase in dermatitis disease intensity) in back skin, when compared with MC-903 vehicle control (FIG. 12C). The overall effects of Compound 2 in preclinical model of atopic dermatitis strongly suggests Compound 2 as a potential clinical candidate for testing in atopic dermatitis indication.

### Example 13

### Compound 2 Significantly Reduces Dermatitis Severity Score in the DNFB Model of Atopic Dermatitis

Experimental details: Preclinical animal model of atopic dermatitis was developed by 2,4-dinitrofluorobenzene (DNFB), a Hapten. In the current study, the test item Compound 2 was screened to evaluate its efficacy in DNFB induced AD model. 0.15% of DNFB was topically applied to 7-8 weeks old female BALB/c mouse from G2-G7 group at a volume of 100 μl/mouse on back skin once in 3 days to induced disease. For the G1 group, (Vehicle control) the same volume of acetone and olive oil (3:1) that was used to solubilize DNFB was applied. Dexamethasone (5 mg/kg, p.o, qd) was dosed per oral once a day to G3 group animals. Compound 2 was administered twice a day to respective treatment groups (G4-G6) for 14 days. On the day of DNFB administration, test compounds were dosed one hour prior to DNFB insult. Clinical scores to assess the disease intensity were measured from visual examination for erythema, scaling, excoriation, and back skin thickness using dial caliper. All the scores were carried by blinded observers to treatment groups. Cumulative dermatitis severity scores were calculated by adding up the individual scores of erythema, scaling, thickness & excoriation. On day-13 of the study, four hours after the last DNFB challenge, scratching behavior was recorded using a web camera in acrylic observational chambers. The number of scratches were measured for 10 minutes. Later, all the animals were terminally bled under mild anesthesia followed by humane sacrifice and back skin tissue was dissected. Required skin tissue samples were collected in fixative and part of the tissue was flash-frozen in liquid nitrogen and stored at −80 °C. Collected tissue samples were processed and used for biomarker analysis using gene and protein expression analysis by qPCR & ELISA, pharmacokinetic analysis by LC-MS and histopathology studies.

Results & Discussion: DNFB induces the activation and proliferation of epidermal keratinocytes and promotes the expression of thymic stromal lymphopoietin (TSLP) expression, a master switch for Th2 allergic inflammation, including AD. This preclinical mouse model utilizes the topical application of DNFB on the skin, which induces skin and systemic AD-like inflammation. The phenotypes closely mimic most of the clinical, skin barrier-related, histological, and immunological characteristics observed in AD patients. Other AD characteristics, such as skin eczematous-like lesions with xerosis and pruritus, increased serum IgE levels, inflammatory infiltrates in the skin and draining lymph nodes, were also observed in mice treated with DNFB. Thus, the DNFB-induced AD mouse model is a promising preclinical research tool for studying immunologic abnormalities involved in AD development or maintenance or preclinical studies for unravelling new therapeutic strategies for this disease.

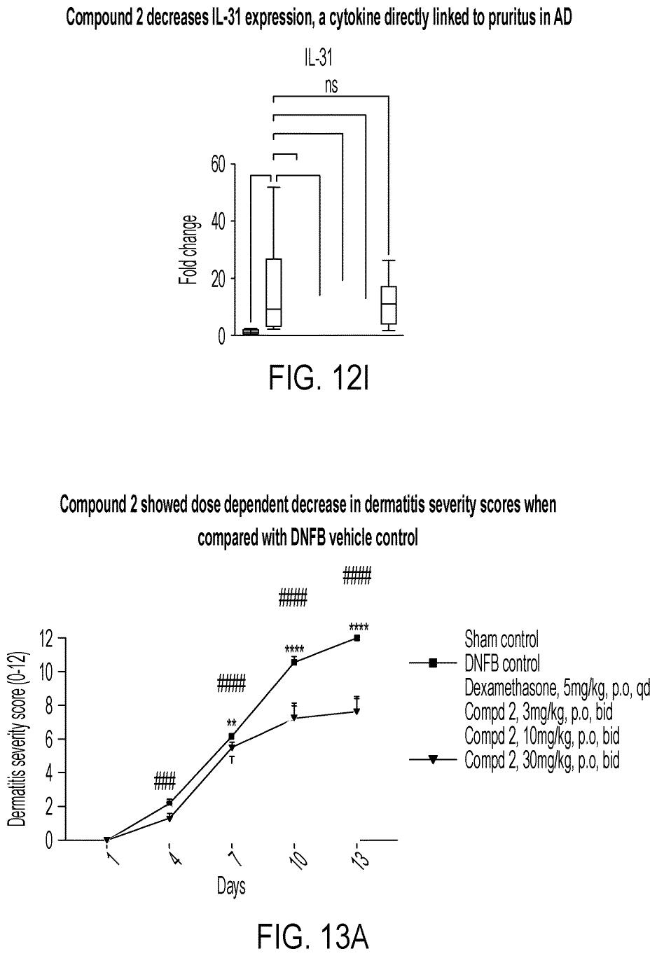

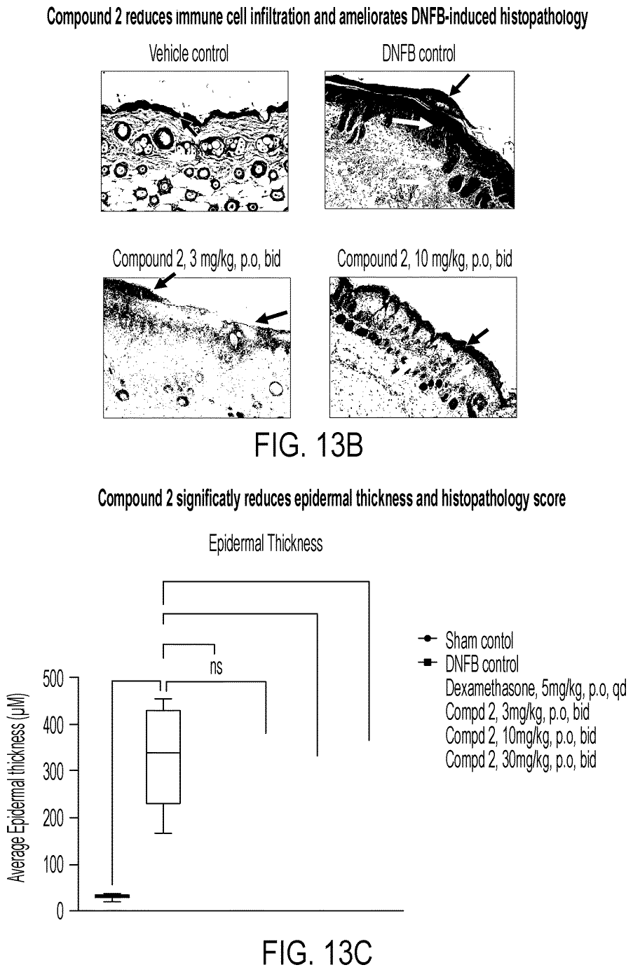

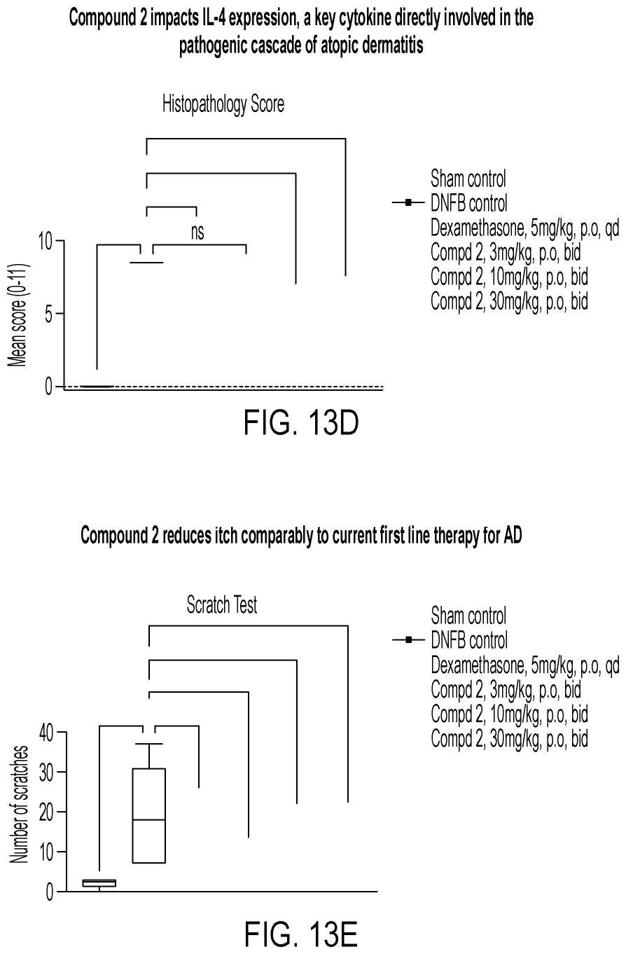

In the current study, DNFB topical application on mouse back skin significantly increased back fold skin thickness and dermatitis severity scores during in-life phase of the study in all animals except in normal vehicle group from D-1 to D-14. Compound 2 showed dose dependent decrease in dermatitis severity scores when compared with DNFB vehicle control (FIG. 13A).

DNFB topical insult significantly increased the epidermal thickness (FIG. 13C) and histopathological changes in keratin, epidermis and dermal skin layers such as immune cell infiltration, hyperkeratosis, parakeratosis, lengthening and clubbing of rete ridges and final histopathology scores (an increase in score means increase in dermatitis disease intensity) in back skin, when compared with normal control (FIG. 13B). DNFB insult also increased the expression of Th2 cytokine IL-4 (FIG. 13D) and number of scratches in scratch test (FIG. 13E) when compared with normal vehicle control.

Compound 2 prophylactic treatment significantly reduced the epidermal thickness (FIG. 13C) and histopathological changes in keratin, epidermis and dermal skin layers such as immune cell infiltration, hyperkeratosis, parakeratosis, lengthening and clubbing of rete ridges and final histopathology scores (an increase in score means increase in dermatitis disease intensity) in back skin, when compared with normal control (FIG. 13B). Compound 2 treatment also reduced the expression of Th2 cytokine IL-4 (FIG. 13D) and number of scratches in scratch test (FIG. 13E) when compared with DNFB vehicle control. The overall effects of Compound 2 in preclinical model of atopic dermatitis suggests Compound 2 can be a clinical candidate for testing in atopic dermatitis indication.

### Example 14

### Compound 2 Shows Similar Effect to Topical JAK-i and High Potency Steroid in the MC903 Model of Atopic Dermatitis Even in Therapeutic Treatment Mode

Experimental details: Preclinical animal model of atopic dermatitis was developed by MC-903, a vitamin-D analogue. In the current study, the test item Compound 2 was screened in therapeutic mode, to evaluate its efficacy in MC-903 induced AD model. 2 nmol of MC-903 was topically applied to 8-10 weeks old female BALB/c mouse from G2-G8 group at a volume of 50 μl/mouse on back skin on every alternate day for 14 days to induced disease. For the G1 group, (Normal+ethanol control) the same volume of ethanol that was used to solubilize MC-903 was applied. Clobetasol (0.05%, 15 mg/mouse) was applied topically once a day to G3 group animals and Ruxolitinib (1.5%, 20 mg/mouse) was applied topically twice a day to G4 group animals. Compound 2 was administered twice a day to respective treatment groups (G4-G8) for 14 days. On the day of MC-903 administration, test compounds were dosed one hour prior to MC-903 insult. Clinical scores to assess the disease intensity were measured from visual examination for erythema, scaling and back skin thickness using dial caliper. All the scores were carried by blinded observers to treatment groups. Disease severity was assessed by dermatitis erythema & dermatitis scaling.

Results & Discussion: MC-903 induces the activation and proliferation of epidermal keratinocytes and promotes the expression of thymic stromal lymphopoietin (TSLP) expression, a master switch for Th2 allergic inflammation, including AD. This preclinical mouse model utilizes the topical application of MC903 on the skin, which induces skin and systemic AD-like inflammation. The phenotypes closely mimic most of the clinical, skin barrier-related, histological, and immunological characteristics observed in AD patients. Other AD characteristics, such as skin eczematous-like lesions with xerosis and pruritus, increased serum IgE levels, inflammatory infiltrates in the skin and draining lymph nodes, were also observed in mice treated with MC-903. Thus, the MC903-induced AD mouse model is a promising preclinical research tool for studying immunologic abnormalities involved in AD development or maintenance or preclinical studies for unravelling new therapeutic strategies for this disease.

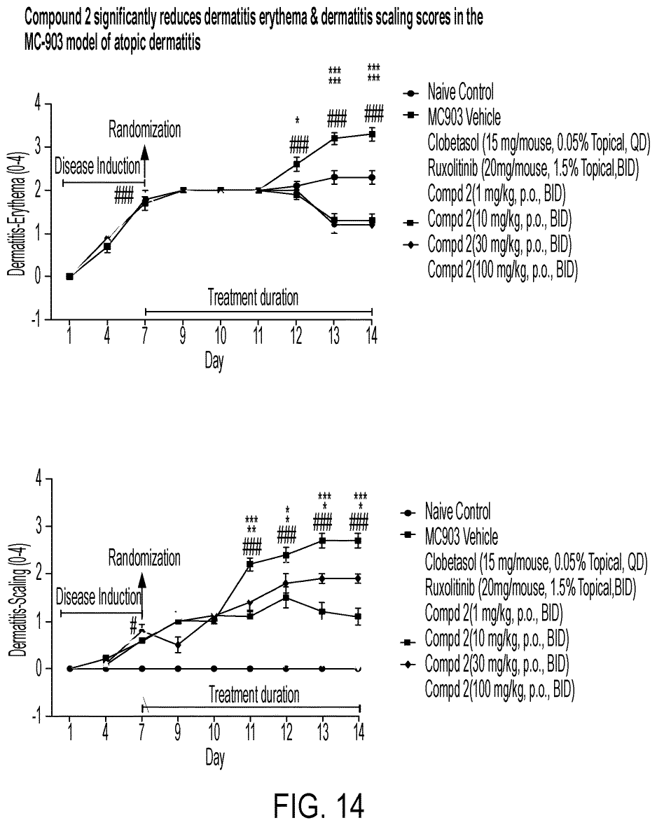

In the current study, MC-903 topical application on mouse back skin significantly increased dermatitis erythema and dermatitis scaling scores during in-life phase of the study in all animals except in normal control group from D-1 to D-14. Compound 2 treatment showed dose dependent decrease in dermatitis erythema and dermatitis scaling scores when compared with MC-903 vehicle control (FIG. 14).

### Example 15

Compound 2 Demonstrates Efficacy On-Par with Oral JAK-i in the MC903 AD Model, with the Potential for an Improved Safety Profile

Experimental details: Preclinical animal model of atopic dermatitis was developed by MC-903, a vitamin-D analogue. In the current study, the test item Compound 2 was screened in therapeutic mode, to evaluate its efficacy in MC-903 induced AD model. 2 nmol of MC-903 was topically applied to 8-10 weeks old female BALB/c mouse from G2-G8 group at a volume of 50 μl/mouse on back skin on every alternate day for 14 days to induced disease. For the G1 group, (Normal+ethanol control) the same volume of ethanol that was used to solubilize MC-903 was applied. Clobetasol (0.05%, 15 mg/mouse) was applied topically once a day to G3 group animals, Upadacitinib was dosed oral twice a day at 2, 6 & 20 mg/kg to G4, G5 & G6 respectively. Compound 2 was administered twice a day to respective treatment groups (G7-G10) for 14 days. On the day of MC-903 administration, test compounds were dosed one hour prior to MC-903 insult. Clinical scores to assess the disease intensity were measured from visual examination for erythema, scaling and back skin thickness using dial caliper. All the scores were carried by blinded observers to treatment groups. Disease severity was assessed by dermatitis severity scores.

Results & Discussion: MC-903 induces the activation and proliferation of epidermal keratinocytes and promotes the expression of thymic stromal lymphopoietin (TSLP) expression, a master switch for Th2 allergic inflammation, including AD. This preclinical mouse model utilizes the topical application of MC903 on the skin, which induces skin and systemic AD-like inflammation. The phenotypes closely mimic most of the clinical, skin barrier-related, histological, and immunological characteristics observed in AD patients. Other AD characteristics, such as skin eczematous-like lesions with xerosis and pruritus, increased serum IgE levels, inflammatory infiltrates in the skin and draining lymph nodes, were also observed in mice treated with MC-903. Thus, the MC903-induced AD mouse model is a promising preclinical research tool for studying immunologic abnormalities involved in AD development or maintenance or preclinical studies for unravelling new therapeutic strategies for this disease.

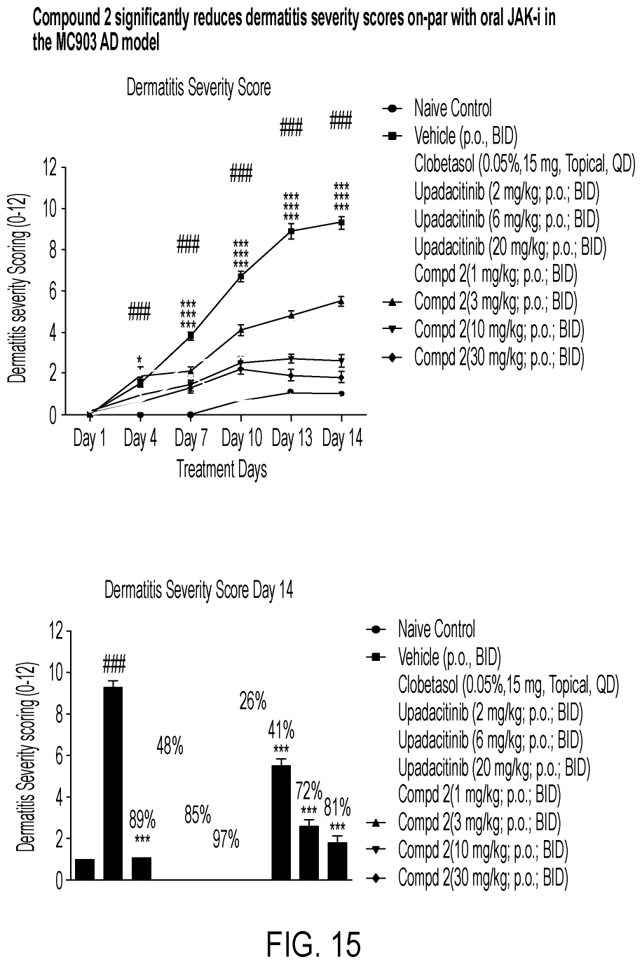

In the current study, MC-903 topical application on mouse back skin significantly increased dermatitis severity scores during in-life phase of the study in all animals except in normal control group from D-1 to D-14. Compound 2 treatment across doses showed significant dose dependent decrease in dermatitis severity scores on-par with oral JAK-i when compared with MC-903 vehicle control (FIG. 15).

### Example 16

Compound 2 is Pharmacologically Active at Doses as Low as 0.1 mg/Kg in the MC903 Model of AD

Experimental details: Preclinical animal model of atopic dermatitis was developed by MC-903, a vitamin-D analogue. In the current study, the test item Compound 2 was screened in therapeutic mode, to evaluate its efficacy in MC-903 induced AD model. 2 nmol of MC-903 was topically applied to 8-10 weeks old female BALB/c mouse from G2-G8 group at a volume of 50 μl/mouse on back skin on every alternate day for 14 days to induced disease. For the G1 group, (Normal+ethanol control) the same volume of ethanol that was used to solubilize MC-903 was applied. Clobetasol (0.05%, 15 mg/mouse) was applied topically once a day to G3 group animals. Compound 2 was administered per oral twice a day at 0.01, 0.1, 1, 3 & 10 mg/kg to respective treatment groups (G4-G8) for 14 days. On the day of MC-903 administration, test compounds were dosed one hour prior to MC-903 insult. Clinical scores to assess the disease intensity were measured from visual examination for erythema, scaling and back skin thickness using dial caliper. All the scores were carried by blinded observers to treatment groups. Disease severity was assessed by dermatitis severity scores.

Results & Discussion: MC-903 induces the activation and proliferation of epidermal keratinocytes and promotes the expression of thymic stromal lymphopoietin (TSLP) expression, a master switch for Th2 allergic inflammation, including AD. This preclinical mouse model utilizes the topical application of MC903 on the skin, which induces skin and systemic AD-like inflammation. The phenotypes closely mimic most of the clinical, skin barrier-related, histological, and immunological characteristics observed in AD patients. Other AD characteristics, such as skin eczematous-like lesions with xerosis and pruritus, increased serum IgE levels, inflammatory infiltrates in the skin and draining lymph nodes, were also observed in mice treated with MC-903. Thus, the MC903-induced AD mouse model is a promising preclinical research tool for studying immunologic abnormalities involved in AD development or maintenance or preclinical studies for unravelling new therapeutic strategies for this disease.

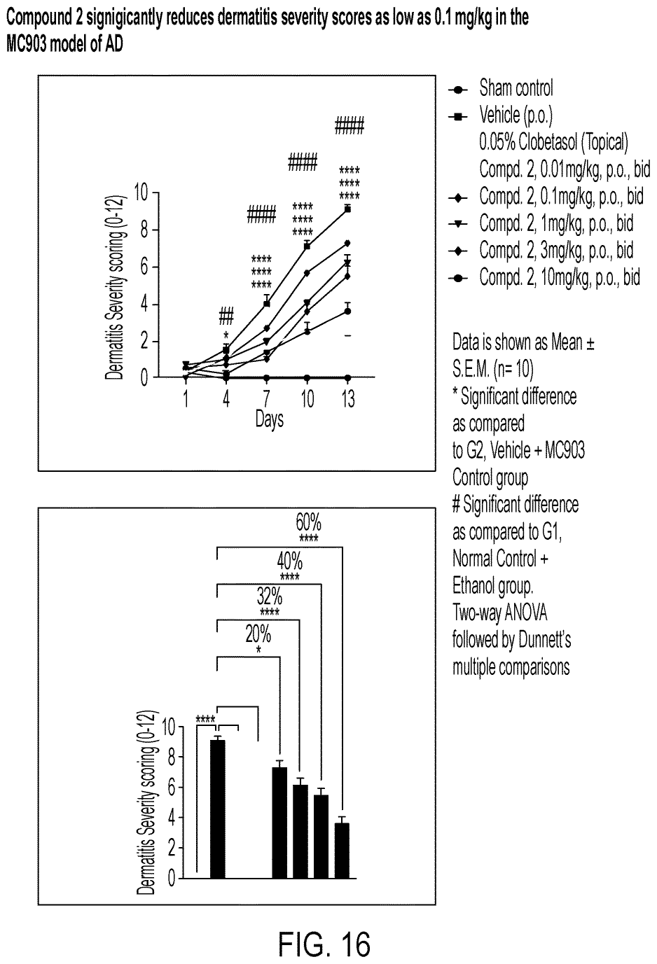

In the current study, MC-903 topical application on mouse back skin significantly increased dermatitis severity scores during in-life phase of the study in all animals except in normal control group from D-1 to D-14. Compound 2 treatment showed significant dose dependent decrease in dermatitis severity scores from the lowest dose of 0.1 mg/kg, p.o, when compared with MC-903 vehicle control (FIG. 16).

1. A compound of Formula (I) or Formula (II): 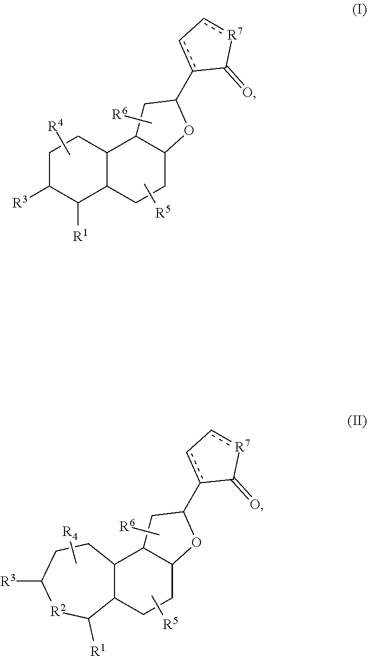
or a stereoisomer thereof, or pharmaceutically acceptable salt of the foregoing, wherein:
R¹ is a C₁-C₆ alkyl, —CN, —NH₂, —OH, —CH₂OH, —CHO, —CH₂R⁸, —COOH, —CONH₂, —COR⁸, —N₃, —SH—SMe, a halogen atom, aryl, heteroaryl, or linked to R³ to form a 3- to 10-membered cyclic, heterocyclic, or heteroaromatic group, wherein the C₁-C₆ alkyl, the aryl, the heteroaryl, and the cyclic or heterocyclic group are unsubstituted or substituted with one or more oxo or R⁹;
R² is oxygen or NR⁹;
R³ is a hydroxyl, ═O, ═N—OH, halogen, —SH, —SMe, —NR⁹R¹⁰, —COOH, —COOR⁹, —OCOR⁹, —CONH₂, —CN, —N₃, C₁-C₅ haloalkyl, or C₁-C₅ alkoxy;
R⁴, R⁵, and R⁶ are each, independently, hydrogen or a C₁-C₅ unsubstituted or substituted alkyl;
R⁷ is oxygen, nitrogen, or NR⁹;
each R⁸ is, independently, a hydroxyl, a halogen, —SH, —SMe, —NR⁹R¹⁰, —COOH, —COOR⁹, —OCOR⁹, —CONH₂, —COR⁹, —COHR⁹R¹⁰, —CN, —N₃, C₁-C₅ alkyl, C₁-C₅ haloalkyl, C₂-C₅ alkenyl, C₂-C₅ alkynyl, C₁-C₅ alkoxy, aryl, aralkyl, or heterocyclyl; and
R⁹ and R¹⁰ are each, independently, hydrogen, C₃-C₈ cycloalkyl, or C₁-C₅ unsubstituted or substituted alkyl;
with the proviso that the compound of Formula (I) is not isoandrographolide.

2. The compound of claim 1, wherein the compound of Formula (I) is Formula (Ia) and the compound of Formula (II) is Formula (IIa): 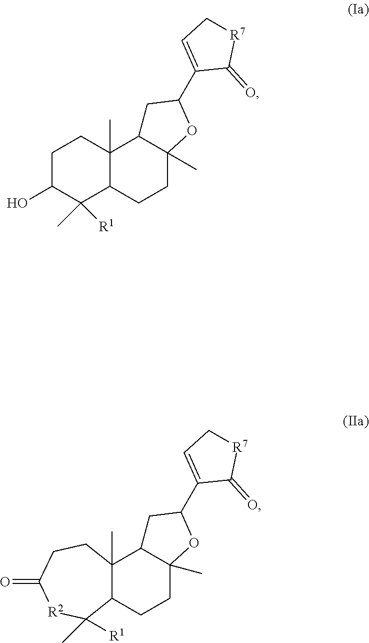
or a stereoisomer thereof, or pharmaceutically acceptable salt of the foregoing, wherein:
R¹ is a C₁-C₆ alkyl, —CN, —NH₂, —OH, —CH₂OH, CHO, —CH₂R⁸, —COOH, —CONH₂, —COR⁸, —N₃, —SH—SMe, a halogen atom, aryl, or heteroaryl, wherein the C₁-C₆ alkyl, the aryl, and the heteroaryl are unsubstituted or substituted with one or more R⁸;
R² is oxygen or NR⁹;
R⁷ is oxygen, nitrogen, or NR⁹;
each R⁸ is, independently, a hydroxyl, a halogen, —SH, —SMe, —NR⁹R¹⁰, —COOH, —COOR⁹, —CONH₂, —COR⁹, —COHR⁹R¹⁰, —CN, —N₃, C₁-C₅ alkyl, C₁-C₅ haloalkyl, C₂-C₅ alkenyl, C₂-C₅ alkynyl, C₁-C₅ alkoxy, aryl, or aralkyl; and
R⁹ and R¹⁰ are each, independently, hydrogen or a C₁-C₅ unsubstituted or substituted alkyl;
with the proviso that the compound of Formula (Ia) is not isoandrographolide.

3. The compound of claim 2, wherein the compound of Formula (Ia) is Formula (Iaa) and the compound of Formula (IIa) is Formula (IIaa): 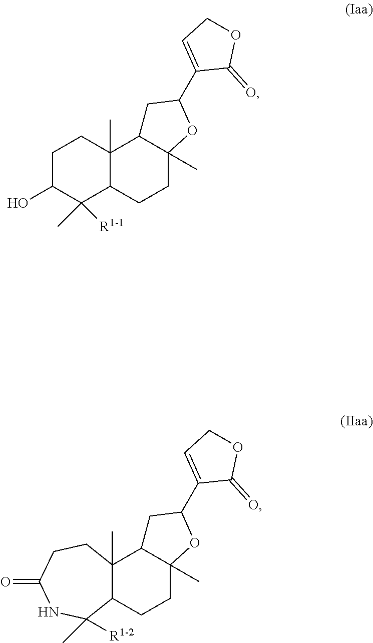
or a stereoisomer thereof, or pharmaceutically acceptable salt of the foregoing, wherein:
R¹⁻¹ is-CN, —NH₂, —OH, —COOH, CHO, —CH₂R⁸, —CONH₂, —COR⁸, —N₃, —SH, —SMe, a halogen atom, aryl, or heteroaryl, wherein the aryl and the heteroaryl are unsubstituted or substituted with one or more R⁸;
R¹⁻² is a C₁-C₆ alkyl, —CN, —NH₂, —OH, —CH₂OH, —COOH, CHO, —CH₂R⁸, —CONH₂, —COR⁸, —N₃, —COR⁸, —SH, —SMe, a halogen atom, aryl, or heteroaryl, wherein the C₁-C₆ alkyl, the aryl, and the heteroaryl are unsubstituted or substituted with one or more R⁸;
each R⁸ is, independently, a hydroxyl, a halogen, —SH, —SMe, —NR⁹R¹⁰, —COOH, —COOR⁹, —CONH₂, —COR⁹, —COHR⁹R¹⁰, —CN, —N₃, C₁-C₅ alkyl, C₁-C₅ haloalkyl, C₂-C₅ alkenyl, C₂-C₅ alkynyl, C₁-C₅ alkoxy, aryl, or aralkyl; and
R⁹ and R¹⁰ are each, independently, hydrogen or a C₁-C₅ unsubstituted or substituted alkyl.

4. The compound of claim 3, or a stereoisomer thereof, or pharmaceutically acceptable salt of the foregoing, wherein the compound is of Formula (Iaa).

5. The compound of claim 4, or a stereoisomer thereof, or pharmaceutically acceptable salt of the foregoing, wherein R¹⁻¹ is-CN.

6. The compound of claim 5, wherein the compound of Formula (Iaa) is: 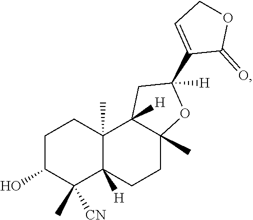
or a pharmaceutically acceptable salt.

7. The compound of claim 4, wherein the compound is of Formula (IIaa), or a stereoisomer thereof, or pharmaceutically acceptable salt of the foregoing.

8. The compound of claim 7, wherein the compound of Formula (IIaa) is: 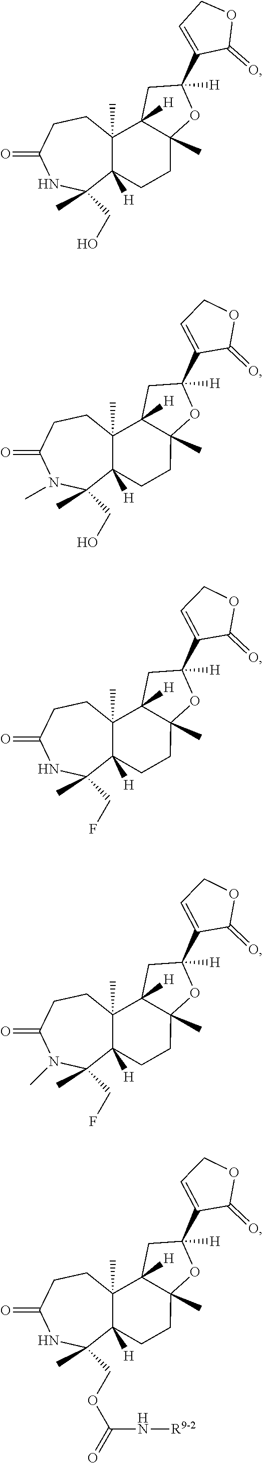 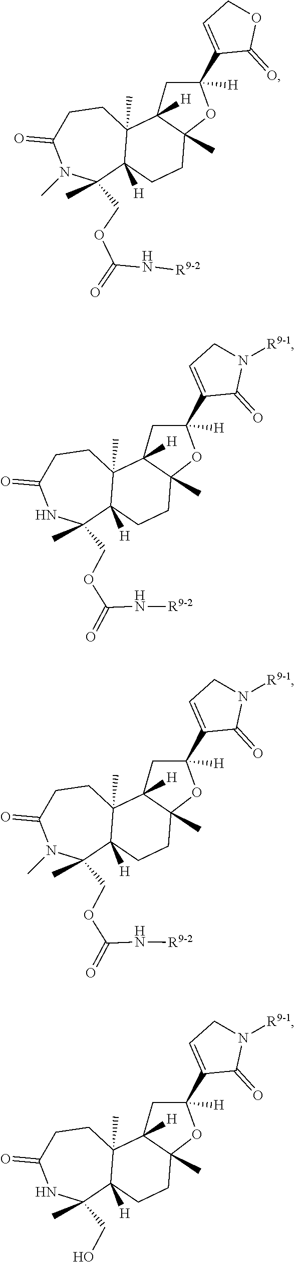 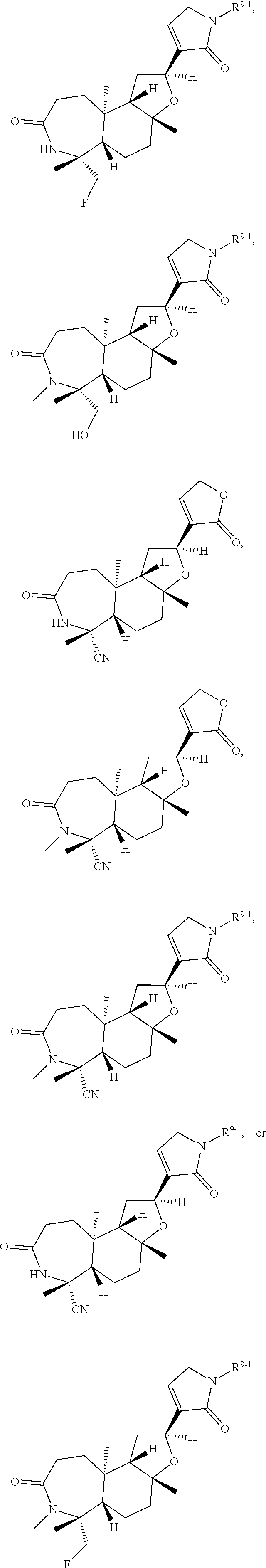
or a stereoisomer thereof, or pharmaceutically acceptable salt of the foregoing, wherein:
R⁹⁻¹ is hydrogen or methyl; and
R⁹⁻² is methyl, ethyl, propyl, isopropyl, cyclopropyl, butyl, secbutyl, or cyclobutyl.

9. The compound of claim 8, or a stereoisomer thereof, or pharmaceutically acceptable salt of the foregoing, wherein R¹⁻² is —CH₂OH or —CN.

10. The compound of claim 9, wherein the compound is: 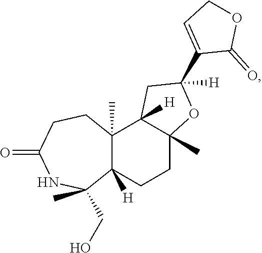
or a pharmaceutically acceptable salt.

11. The compound of claim 9, wherein the compound is: 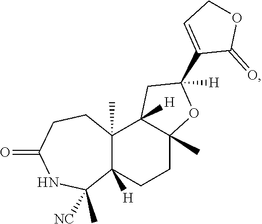
or a pharmaceutically acceptable salt.

12. A method of making a compound of claim 4, wherein the compound is of Formula (Iaa), the method comprising:
providing a solution of isoandrographolide and a solvent;
combining 2,2,6,6-tetramethylpiperidinyloxy (TEMPO), tetrabutylammonium iodide (TBAI), N-Chlorosuccinimide (NCS), a salt, and the solution to form a first intermediate;
combining hydroxylamine hydrochloride, pyridine, and the first intermediate to form a second intermediate; and
dehydration into cyanide of the second intermediate to form the compound of Formula (Iaa).

13. The method of claim 12, wherein the step of dehydration into cyanide, comprises combining triethylamine, 4-toluenesulfonyl chloride, and the second intermediate to form the compound of Formula (Iaa).

14. The method of claim 12, wherein the step of dehydration into cyanide, comprises combining benzotriazol-1-yloxytripyrrolidinophosphonium hexafluorophosphate (PyBOP), 1,8-Diazabicyclo[5.4.0]undec-7-ene (DBU), and the second intermediate to form the compound of Formula (Iaa).

15. A method of making a compound of claim 4, wherein the compound is of Formula (IIaa), the method comprising:
providing a solution of isoandrographolide and a solvent;
combining tert-Butyldimethylsilyl chloride (TBDMSCl), and the solution to form a first intermediate;
combining pyridine, celite, pyridinium chlorochromate, and the first intermediate to form a second intermediate;
combining hydroxylamine hydrochloride, pyridine, and the second intermediate to form a third intermediate; and
combining 4-toluenesulfonyl chloride and the third intermediate followed by treatment with dioxane HCL to form the compound of Formula (IIaa).

16. A method of making a compound of claim 4, wherein the compound is of Formula (IIaa), the method comprising:
providing a solution of isoandrographolide and a solvent;
combining Dess-Martin Periodinane (DMP), and the solution to form a first intermediate;
combining hydroxylamine hydrochloride, pyridine, and the first intermediate to form a second intermediate; and
combining triethylamine, 4-methylbenzene-1-sulfonyl chloride, 4-dimethylaminopyridine (DMAP), and the second intermediate, followed by treatment with dioxane HCl to form the compound of Formula (IIaa).

17. A pharmaceutical composition comprising the compound of claim 1, or a stereoisomer thereof, or pharmaceutically acceptable salt of the foregoing, and at least one pharmaceutically acceptable carrier.

18. A method of treating, or preventing a neutrophil-related condition or disease in a subject in need thereof, comprising administering to the subject a therapeutically effective amount of a compound according to claim 1, or a stereoisomer thereof, or pharmaceutically acceptable salt of the foregoing.

19. The method of claim 18, wherein the condition or disease is a cardiovascular condition or disease, an endocrine condition or disease, a neurological condition or disease, a gastrointestinal condition or disease, or a dermatological condition or disease, or any combination thereof.

20. The method of claim 18, wherein the method of treating is a method of ameliorating a neutrophil-related condition or disease in the subject.

21. A pharmaceutical composition comprising a compound of claim 1, or a stereoisomer thereof, or pharmaceutically acceptable salt of the foregoing, for use in a method of treating, or preventing a neutrophil-related condition or disease.

22. The pharmaceutical composition of claim 21, wherein the condition or disease is a cardiovascular condition or disease, an endocrine condition or disease, a neurological condition or disease, a gastrointestinal condition or disease, or a dermatological disease or condition, or any combination thereof.

23. Use of a compound of claim 1, or a stereoisomer thereof, or pharmaceutically acceptable salt of the foregoing, in the preparation of a medicament for use in treating, or preventing a neutrophil-related condition or disease.

24. The use of claim 22, wherein the condition or disease is a cardiovascular condition or disease, an endocrine condition or disease, a neurological condition or disease, a gastrointestinal condition or disease, or a dermatological disease or condition, or any combination thereof.

25. A method of treating, or preventing a fibrotic condition or disease in a subject in need thereof, comprising administering to the subject a therapeutically effective amount of a compound according to claim 1, or a stereoisomer thereof, or pharmaceutically acceptable salt of the foregoing.

26. The method of claim 25, wherein the condition or disease is idiopathic pulmonary fibrosis (IPF).

27. The method of claim 25, wherein the method of treating is a method of ameliorating a fibrotic condition or disease in the subject.

28. A pharmaceutical composition comprising a compound of claim 1, or a pharmaceutically acceptable salt or stereoisomer thereof, for use in a method of treating, or preventing a fibrotic condition or disease.

29. The pharmaceutical composition of claim 28, wherein the condition or disease is idiopathic pulmonary fibrosis (IPF).

30. Use of a compound of claim 1, or a stereoisomer thereof, or pharmaceutically acceptable salt of the foregoing, in the preparation of a medicament for use in treating, or preventing a fibrotic condition or disease.

31. The use of claim 30, wherein the condition or disease is idiopathic pulmonary fibrosis (IPF).

32. A method of treating, or preventing a dermatological condition or disease in a subject in need thereof, comprising administering to the subject a therapeutically effective amount of a compound according to claim 1, or a stereoisomer thereof, or pharmaceutically acceptable salt of the foregoing.

33. The method of claim 32, wherein the condition or disease is atopic dermatitis.

34. The method of claim 32, wherein the method of treating is a method of ameliorating a dermatological condition or disease in the subject.

35. A pharmaceutical composition comprising a compound of claim 1, or a pharmaceutically acceptable salt or stereoisomer thereof, for use in a method of treating, or preventing a dermatological condition or disease.

36. The pharmaceutical composition of claim 35, wherein the condition or disease is atopic dermatitis.

37. Use of a compound of claim 1, or a pharmaceutically acceptable salt or stereoisomer thereof, in the preparation of a medicament for use in treating, or preventing a dermatological condition or disease.

38. The use of claim 37, wherein the condition or disease is atopic dermatitis.

39. The method of claim 32, wherein the subject is a human.
